(19) United States
(12) Patent Application Publication
(10) Pub. No.: US 2024/0245771 A1
(43) Pub. Date: Jul. 25, 2024

(54) FULLY HUMAN ANTIBODY FOR HUMAN B7H3, CHIMERIC ANTIGEN RECEPTOR AND USES THEREOF

(71) Applicant: XUZHOU MEDICAL UNIVERSITY, Xuzhou (CN)

(72) Inventors: Gang WANG, Xuzhou (CN); Junnian ZHENG, Xuzhou (CN); Huizhong LI, Xuzhou (CN); Bo ZHAO, Xuzhou (CN); Juan LI, Xuzhou (CN); Yilin LIU, Xuzhou (CN); Peiyu CAO, Xuzhou (CN); Xinyu LI, Xuzhou (CN); Liu LIU, Xuzhou (CN)

(21) Appl. No.: 18/401,115

(22) Filed: Dec. 29, 2023

Related U.S. Application Data
(63) Continuation-in-part of application No. PCT/CN2021/115806, filed on Aug. 31, 2021.

(30) Foreign Application Priority Data
Jun. 30, 2021 (CN) 202110736498.8
Jun. 30, 2021 (CN) 202110736533.6
Jun. 30, 2021 (CN) 202110739303.5
Jun. 30, 2021 (CN) 202110739305.4
Jun. 30, 2021 (CN) 202110739693.6
Jun. 30, 2021 (CN) 202110739700.2
Jul. 7, 2021 (CN) 202110768579.6
Jul. 7, 2021 (CN) 202110768590.2
Jul. 7, 2021 (CN) 202110768592.1
Jul. 12, 2021 (CN) 202110783589.7
Jul. 12, 2021 (CN) 202110784331.9

(51) Int. Cl. A61K 39/00; A61P 35/00; C07K 14/52; C07K 14/535; C07K 14/54; C07K 14/55; C07K 14/705; C07K 14/71; C07K 14/715; C07K 14/725; C07K 14/73; C07K 14/735; C07K 16/28; C12N 5/0783; G01N 33/68

(52) U.S. Cl. CPC A61K 39/464411; A61K 39/4631; A61P 35/00; C07K 14/521; C07K 14/535; C07K 14/54; C07K 14/5434; C07K 14/5443; C07K 14/55; C07K 14/7051; C07K 14/70514; C07K 14/70517; C07K 14/70521; C07K 14/70535; C07K 14/70578; C07K 14/70596; C07K 14/71; C07K 14/7155; C07K 16/2827; C12N 5/0636; C12N 5/0646; G01N 33/68; C07K 2317/21; C07K 2319/02; C07K 2319/03; C12N 2510/00; G01N 2333/70532

(57) ABSTRACT

Provided are a novel fully human antibody for human B7H3, a chimeric antigen receptor, and uses thereof; also provided are a novel fully human anti-human B7H3 antibody, a chimeric antigen receptor containing the antibody, and genetically engineered cells expressing the receptor and the antibody. It has been verified by experiments that CAR-T, CAR-NK and CAR-iNKT cells targeting B7H3 prepared on the basis of the present chimeric antigen receptor have relatively strong proliferation ability, cytokine release ability and tumor cell killing ability, and can effectively eliminate tumor cells.

### CROSS-REFERENCE TO RELATED APPLICATIONS

The present application is a continuation-in-part of PCT international application PCT/CN2021/115806, filed on Aug. 31, 2021, which claims priority to the following application documents: application No. 202110736498.8 entitled “NOVEL FULLY HUMAN ANTI-HUMAN B7H3 ANTIBODY, COMPOSITION CONTAINING SAME AND APPLICATION OF ANTIBODY” filed on Jun. 30, 2021, application No. 202110768579.6 entitled “APPLICATION OF SKI IN PREPARATION OF SYNERGISTIC CAR-T CELLS” filed on Jul. 7, 2021, application No.

202110784331.9 entitled “B7H3-TARGETED FULLY-HUMANIZED CHIMERIC ANTIGEN RECEPTOR, INKT CELL AND APPLICATION THEREOF” filed on Jul. 12, 2021, application No. 202110783589.7 entitled “TARGETING B7H3 CO-EXPRESSION IL-21 FULLY HUMAN CHIMERIC ANTIGEN RECEPTOR, INKT CELL AND APPLICATION THEREOF” filed on Jul. 12, 2021, application No. 202110736533.6 entitled “B7H3 SPECIFIC RESISTANCE CAR-NK CELL” filed on Jun. 30, 2021, application No. 202110739700.2 entitled “FULLY HUMANIZED ANTIBODY SCFV TARGETING CD276, CHIMERIC ANTIGEN RECEPTOR, ENGINEERED IMMUNE CELL AND PREPARATION METHOD THEREOF” filed on Jun. 30, 2021, application No. 202110768592.1 entitled “PREPARATION METHOD AND APPLICATION OF FULLY HUMAN CAR-T CELLS TARGETING CD276” filed on Jul. 7, 2021, application No.

202110768590.2 entitled “APPLICATION OF GSTP1 IN PREPARATION OF SYNERGISTIC CAR-T” filed on Jul. 7, 2021, application No. 202110739303.5 entitled “APPLICATION OF SKI IN PREPARATION OF SYNERGISTIC CAR-T CELLS” filed on Jun. 30, 2021, application No. 202110739305.4 entitled “B7H3-TARGETED FULLY-HUMANIZED CHIMERIC ANTIGEN RECEPTOR, INKT CELL AND APPLICATION THEREOF” filed on Jun. 30, 2021, and application No. 202110739693.6 entitled “PREPARATION METHOD AND APPLICATION OF FULLY HUMAN CAR-T CELLS TARGETING CD276” filed on Jun. 30, 2021, the content of each is incorporated herein by reference in its entirety.

### INCORPORATION OF SEQUENCE LISTING

This application contains a sequence listing submitted in Computer Readable Form (CRF). The CFR file containing the sequence listing entitled “PA630.0010.xml”, which was created on Dec. 28, 2023, and is 117,721 bytes in size. The information in the sequence listing is incorporated herein by reference in its entirety.

### TECHNICAL FIELD

The present application belongs to the technical field of immunology and molecular biology, and specifically relates to a novel fully human anti-human B7H3 antibody. More specifically, the present application relates to a novel fully human anti-human B7H3 antibody, a chimeric antigen receptor comprising the antibody, a genetically engineered cell expressing the receptor and the antibody and use thereof in adoptive cell therapy.

### BACKGROUND

Tumor cell immunotherapy is the fourth major tumor treatment technology after surgery, radiotherapy, and chemotherapy, and is a novel treatment method that utilizes biotechnology and biological agents to isolate, activate ex vivo, and reinfuse the patient's own or allogeneic tumor-specific or non-specific killer cells. Traditional therapies for tumors, including surgery, chemotherapy, and radiotherapy, have limitations: Surgery often fails to achieve complete eradication due to the infiltration of cancer cells into adjacent tissues or their metastasis to distant sites; chemotherapy and radiotherapy are limited by their toxicity and damage to other normal tissues within the body. In recent years, popular targeted therapy can design corresponding therapeutic drugs aiming at the definite carcinogenic sites on the cellular molecular level, and the drugs enter the body and can specifically select the carcinogenic sites to combine and act so as to cause the tumor cells to die specifically, but the molecular targeted drugs only can act on specific gene mutant tumors, and if the target tumor genes are mutated, the drug tolerance will be generated, so that the curative effect is reduced, and serious adverse reaction and other problems may occur. Tumor cell immunotherapy is different from traditional therapies. The immune system of a normal human body can recognize and remove tumor cells, but cancer patients, especially advanced cancer patients, often suffer from damage of the immune system, so that the capacity of removing the tumor cells is lost. In this case, the aim of controlling and killing the tumor cells can be achieved by exciting and enhancing the immune function of the organism, and the treatment method is the tumor cell immunotherapy.

In tumor cell immunotherapy, chimeric antigen receptor modified T cell (CAR-T), chimeric antigen receptor modified NK cell (CAR-NK), and chimeric antigen receptor modified iNK cell (CAR-iNK) immunotherapies are the two therapies that are the most rapidly developed at present. The principle of the immunotherapies is that T cells or NK cells modified by a chimeric antigen receptor (CAR) can specifically recognize tumor-associated antigens on the surface of tumor cells, so that the targeting property, the killing activity, and the persistence of the effector T cells or NK cells are higher than those of conventionally applied immune cells. Additionally, these modified cells can overcome the local immunosuppressive microenvironment of the tumor and break the host's immune tolerance. The natural killer T cell (NKT), which is also an immune cell, is different from the traditional T cell or NK cell, is a special T cell with the innate immune response function, combines the functions of NK cells with the characteristics of T cells, and is divided into the type I NKT cell, the type II NKT cell, and the type III NKT cell, wherein the type I NKT cell is also called invariant natural killer T cell (INKT), and is the most extensively and deeply researched type of NKT cells at present. A large number of researches show that the iNKT cell has a relatively good anti-tumor effect and has a huge potential application value in tumor immunotherapy.

B7H3 (CD276) belongs to the B7 superfamily and is a transmembrane glycoprotein. Its extracellular region is structured in 2 forms: One is the monovalent 2Ig-B7-H3, and the other is the bivalent 4Ig-B7-H3, which is composed of 2 repeating unit structures. Related studies have shown that B7H3 can inhibit T cell proliferation and cytokine release by interacting with a receptor of unknown structure (Suh W K, Gajewska B U, Okada H, et al. The B7 family member B7-H3 preferentially down-regulates T helper type 1-mediated immune responses[J]. Nature immunology, 2003, 4(9): 899-906.). Although the receptor for B7H3 is unknown, more and more reports have recently been made on the negative regulation of tumor immunity by the interaction between B7H3 and its receptors. Tumor cells express B7H3, which evade immune surveillance by CD8+ T cells. Related studies have shown that mice with knockout of B7H3 gene or the use of anti-B7H3 antibodies significantly inhibit tumor growth. The inhibitory effect depends on the function of CD8+ T cells and NK cells (Lee Y, Martin-Orozco N, Zheng P, et al. Inhibition of the B7-H3 immune checkpoint limits tumor growth by enhancing cytotoxic lymphocyte function[J]. Cell research, 2017, 27(8): 1034-1045.). The above studies indicate that B7H3 can serve as an effective therapeutic target for cancer immunotherapy.

In view of this, the present application provides a novel fully human anti-human B7H3 antibody, a fully human B7H3-targeting chimeric antigen receptor comprising the antibody, a genetically engineered cell expressing the receptor and the antibody and use thereof in adoptive cell therapy based on the high expression characteristic of B7H3 in tumor cells and the characteristic of the inhibitory activity of B7H3 on immune cells, which have important application prospects in the field of tumor cell immunotherapy.

### SUMMARY

The present application aims at a fully human chimeric antigen receptor targeting B7H3, an iNKT cell and use thereof.

In order to achieve the object described above, the present application adopts the following technical solutions:

In a first aspect of the present application, provided is an isolated fully human monoclonal antibody or an antigen-binding fragment thereof.

Further, the antibody or the antigen-binding fragment thereof specifically binds to B7H3; preferably, the antibody or the antigen-binding fragment thereof comprises an HCVR and an LCVR;
more preferably, the HCVR comprises an HCDR1, an HCDR2 and an HCDR3;
more preferably, the LCVR comprises an LCDR1, an LCDR2 and an LCDR3;
most preferably, the HCDR1, the HCDR2 and the HCDR3 are an HCDR1, an HCDR2 and an HCDR3, respectively, in an HCVR with an amino acid sequence set forth in SEQ ID NO: 7 or SEQ ID NO: 8;
most preferably, the LCDR1, the LCDR2 and the LCDR3 are an LCDR1, an LCDR2 and an LCDR3, respectively, in an LCVR with an amino acid sequence set forth in SEQ ID NO: 17 or SEQ ID NO: 18;
most preferably, the HCDR1 comprises an amino acid sequence set forth in SEQ ID NO: 1 or SEQ ID NO: 2, or an amino acid sequence having at least 95%, at least 96%, at least 97%, at least 98% or at least 99% identity to SEQ ID NO: 1 or SEQ ID NO: 2;
most preferably, the HCDR2 comprises an amino acid sequence set forth in SEQ ID NO: 3 or SEQ ID NO: 4, or an amino acid sequence having at least 95%, at least 96%, at least 97%, at least 98% or at least 99% identity to SEQ ID NO: 3 or SEQ ID NO: 4;
most preferably, the HCDR3 comprises an amino acid sequence set forth in SEQ ID NO: 5 or SEQ ID NO: 6, or an amino acid sequence having at least 95%, at least 96%, at least 97%, at least 98% or at least 99% identity to SEQ ID NO: 5 or SEQ ID NO: 6;
most preferably, the LCDR1 comprises an amino acid sequence set forth in SEQ ID NO: 11 or SEQ ID NO: 12, or an amino acid sequence having at least 95%, at least 96%, at least 97%, at least 98% or at least 99% identity to SEQ ID NO: 11 or SEQ ID NO: 12;
most preferably, the LCDR2 comprises an amino acid sequence set forth in SEQ ID NO: 13 or SEQ ID NO: 14, or an amino acid sequence having at least 95%, at least 96%, at least 97%, at least 98% or at least 99% identity to SEQ ID NO: 13 or SEQ ID NO: 14;
most preferably, the LCDR3 comprises an amino acid sequence set forth in SEQ ID NO: 15 or SEQ ID NO: 16, or an amino acid sequence having at least 95%, at least 96%, at least 97%, at least 98% or at least 99% identity to SEQ ID NO: 15 or SEQ ID NO: 16;
most preferably, the HCVR of the antibody or the antigen-binding fragment thereof has an amino acid sequence set forth in SEQ ID NO: 7 or SEQ ID NO: 8;
most preferably, the LCVR of the antibody or the antigen-binding fragment thereof has an amino acid sequence set forth in SEQ ID NO: 17 or SEQ ID NO: 18;
most preferably, the HCVR of the antibody or the antigen-binding fragment thereof and the LCVR of the antibody or the antigen-binding fragment thereof are linked by a Linker;
most preferably, the Linker has an amino acid sequence set forth in SEQ ID NO: 21 or SEQ ID NO: 22;
most preferably, the antibody or the antigen-binding fragment thereof has an amino acid sequence set forth in SEQ ID NO: 25 or SEQ ID NO: 26.

The present application further provides an antibody-drug conjugate.

Further, the antibody-drug conjugate comprises the antibody or the antigen-binding fragment thereof according to the first aspect of the present application;
preferably, the antibody-drug conjugate further comprises a small molecule drug;
more preferably, the antibody-drug conjugate is formed by covalent attachment of the antibody or the antigen-binding fragment thereof according to the first aspect of the present application to a small molecule drug;
more preferably, the small molecule drug includes an alkylating agent, an antimetabolite, an anti-tumor antibiotic, a mitotic inhibitor, a chromatin function inhibitor, an anti-angiogenic agent, an antiestrogen, an antiandrogen, and an immunomodulator;
most preferably, the alkylating agent includes dichloroethylmethylamine, chlorambucil, melphalan, bromopropylpiperazine, prednimustine, estramustine phosphate, cyclophosphamide, hexamethylmelamine, trofosfamide, ifosfamide, thiotepa, carmustine, streptozocin, fotemustine, lomustine, busulfan, treosulfan, improsulfan, dacarbazine, cisplatin, oxaliplatin, and carboplatin;
most preferably, the antimetabolite includes methotrexate, 5-fluorouracil, fluoronucleoside, 5-fluorodeoxyuracil, capecitabine, cytarabine, fludarabine, cytarabine, 6-mercaptopurine (6-MP), 6-mercaptoguanine (6-TG), 2-chlorodeoxyadenosine, 5-azacytidine, 2,2-difluorodeoxycytidine, cladribine, deoxycoformycin, and pentostatin;
most preferably, the anti-tumor antibiotic includes doxorubicin, daunorubicin, idarubicin, valrubicin, mitoxantrone hydrochloride, dactinomycin, mithramycin, mithramycin, mitomycin C, bleomycin, and procarbazine;
most preferably, the mitotic inhibitor includes paclitaxel, docetaxel, vinblastine, vincristine, vindesine, and vinorelbine;
most preferably, the chromatin function inhibitor includes topotecan, irinotecan, etoposide, etoposide phosphate, and teniposide;
most preferably, the anti-angiogenic agent includes razoxane, marimastat, batimastat, prinomastat, tanomastat, ilomastat, CGS-27023A, halofuginone, COL-3, neovastat, BMS-275291, and thalidomide;
most preferably, the antiestrogen includes tamoxifen, toremifene, raloxifene, droloxifene, idoxifene, anastrozole, letrozole, and exemestane;
most preferably, the antiandrogen includes flutamide, nilutamide, bicalutamide, spironolactone, cyproterone acetate, finasteride, and cimetidine;
most preferably, the immunomodulator includes interferon, interleukin, tumor necrosis factor, lentinan, sizofiran, roquinimex, pidotimod, pegademase, and thymosin formulations.

The antibody of the present application may be any type of immunoglobulin known in the art. For example, the anti-CD276 binding moiety may be an antibody of any isotype, e.g., IgA, IgD, IgE, IgG (e.g., IgG1, IgG2, IgG3, or IgG4), and IgM. The antibody may be monoclonal or polyclonal. The antibody may be a naturally occurring antibody, e.g., an antibody isolated and/or purified from a mammal, e.g., a mouse, a rabbit, a goat, a horse, a chicken, a hamster, and a human. Alternatively, the antibody may be a genetically-engineered antibody, e.g., a humanized antibody, a fully human antibody, and a chimeric antibody. The antibody may be in monomeric or polymeric form. Moreover, the antibody may have any level of affinity for CD276.

Methods for testing the ability of an antibody to bind to CD276 are known in the art and include any antibody-antigen binding assay, such as radioimmunoassay (RIA), enzyme-linked immunosorbent assay (ELISA), western blot, immunoprecipitation, competitive inhibition assay, and competitive inhibition assay.

Suitable methods for preparing antibodies are known in the art. For example, standard hybridoma methods. In addition, other methods may be used, for example, phage vector expression systems, which are known in the art. Methods for producing antibodies in non-human animals are found, for example, in U.S. Pat. Nos. 5,545,806, 5,569,825, and 5,714,352, and U.S. Patent Application Publication No. 2002/0197266 A1.

Further, the antibody includes a full-length antibody and an antigen-binding fragment of a full-length antibody.

Further, the antibody is a fully human antibody.

Further, the antigen-binding fragment includes IgG, Fab, Fab′, F(ab′)2, Fv, scFv, and single-domain antibodies;

In a specific embodiment of the present application, the antigen-binding fragment is an scFv.

Single-chain variable fragment (scFv) antibody fragments, which are truncated Fab fragments, may be generated using methods that comprises linking the light chain variable domain of an antibody to the heavy chain variable domain of the antibody by a synthetic peptide. Disulfide-stabilized variable region fragments (dsFvs) may be prepared by recombinant DNA techniques using conventional recombinant DNA techniques (see, e.g., Reiter et al., Protein Engineering 7:697-704 (1994)).

In a second aspect of the present application, provided is a fully human chimeric antigen receptor targeting B7H3.

Further, the chimeric antigen receptor comprises the antibody or the antigen-binding fragment thereof according to the first aspect of the present application;
preferably, the chimeric antigen receptor further comprises a transmembrane domain;
preferably, the chimeric antigen receptor further comprises an intracellular signaling domain;
preferably, the chimeric antigen receptor further comprises a hinge region;
preferably, the chimeric antigen receptor further comprises a signal peptide;
preferably, the chimeric antigen receptor further comprises a co-stimulatory signaling domain;
more preferably, the transmembrane domain comprises transmembrane domains of the following molecules: CD8α, CD28, IgG1, IgG4, 4-1BB, PD-1, CD34, OX40, CD3ε, IL-2 receptor, IL-7 receptor, and IL-11 receptor;
more preferably, the intracellular signaling domain comprises intracellular signaling domains of the following molecules: CD3ζ, FcRγ, FcRβ, CD3γ, CD3ζ, CD3ε, TCRζ, CD4, CD5, CD8, CD21, CD22, CD79a, CD79b, CD278, FcεRI, DAP10, DAP12, CD66d, DAP10, DAP12, and FYN;
more preferably, the hinge region comprises hinge regions of the following molecules: CD8α, CD28, IgG1, IgG4, 4-1BB, PD-1, CD34, OX40, CD3ε, IL-2 receptor, IL-7 receptor, and IL-11 receptor;
more preferably, the signal peptide comprises signal peptides of the following molecules: α and β chains of a T cell receptor, CD3ζ, CD3ε, CD4, CD5, CD8, CD9, CD28, CD16, CD22, CD33, CD37, CD45, CD64, CD80, CD86, CD134, CD137, CD154, GITR, GM-CSF, ICOS, and IgG6;
more preferably, the co-stimulatory signaling domain comprises co-stimulatory signaling domains of the following molecules: CD28, ICOS (CD278), CD27, CD19, CD4, CD8α, CD8β, BAFFR, HVEM, LIGHT, KIRDS2, SLAMF7, NKp80 (KLRF1), NKp30, NKp46, CD40, CDS, ICAM-1, 4-1BB (CD137), B7-H3, OX40, DR3, GITR, CD30, TIM1, CD2, CD7, and CD226;
more preferably, the chimeric antigen receptor is obtained by sequentially connecting a signal peptide, the antibody or the antigen-binding fragment thereof according to the first aspect of the present application, a hinge region, a transmembrane domain, a co-stimulatory signaling domain, and an intracellular signaling domain in series;
most preferably, the transmembrane domain is a transmembrane domain of CD8α;
most preferably, the transmembrane domain of CD8a has an amino acid sequence set forth in SEQ ID NO: 29;
most preferably, the transmembrane domain of CD8a has a nucleotide sequence set forth in SEQ ID NO: 30;
most preferably, the intracellular signaling domain is an intracellular signaling domain of CD3ζ;
most preferably, the intracellular signaling domain of CD3ζ has an amino acid sequence set forth in SEQ ID NO: 31;
most preferably, the intracellular signaling domain of CD3ζ has a nucleotide sequence set forth in SEQ ID NO: 32;
most preferably, the hinge region is a hinge region of CD8α;
most preferably, the hinge region of CD8α has an amino acid sequence set forth in SEQ ID NO: 33;
most preferably, the hinge region of CD8α has a nucleotide sequence set forth in SEQ ID NO: 34;
most preferably, the signal peptide is a signal peptide of IgG6;
most preferably, the signal peptide of IgG6 has an amino acid sequence set forth in SEQ ID NO:

35;
most preferably, the signal peptide of IgG6 has a nucleotide sequence set forth in SEQ ID NO: 36;
most preferably, the co-stimulatory signaling domain is a co-stimulatory signaling domain of CD28 or a co-stimulatory signaling domain of CD137;
most preferably, the co-stimulatory signaling domain of CD28 has an amino acid sequence set forth in SEQ ID NO: 37;
most preferably, the co-stimulatory signaling domain of CD28 has a nucleotide sequence set forth in SEQ ID NO: 3δ;
most preferably, the co-stimulatory signaling domain of CD137 has an amino acid sequence set forth in SEQ ID NO: 39;
most preferably, the co-stimulatory signaling domain of CD137 has a nucleotide sequence set forth in SEQ ID NO: 40;
preferably, the chimeric antigen receptor further comprises a self-cleaving peptide;
preferably, the chimeric antigen receptor further comprises a TGF-β-antagonizing domain;
preferably, the chimeric antigen receptor further comprises a safety switch;
preferably, the chimeric antigen receptor further comprises an immunomodulatory molecule or cytokine;
preferably, the chimeric antigen receptor further comprises an ROS-inhibiting domain;
more preferably, the self-cleaving peptide includes T2A, P2A, E2A, and F2A;
more preferably, the TGF-β-antagonizing domain comprises an antibody specifically binding to TGF-β, and a nucleic acid molecule encoding a TGF-β signaling-inhibiting protein;
more preferably, the safety switch includes tEGFR, iCaspase-9, and RQR8;
more preferably, the immunomodulatory molecule or cytokine includes B7.1, CCL19, CCL21, CD40L, CD137L, GITRL, GM-CSF, IL-12, IL-2, IL-15, IL-18, IL-21, LEC, and OX40L;
more preferably, the ROS-inhibiting domain comprises a nucleic acid molecule encoding an ROS-inhibiting GSTP1 protein;
most preferably, the self-cleaving peptide is T2A;
most preferably, the TGF-β-antagonizing domain is human Ski;
most preferably, the safety switch is tEGFR;
most preferably, the immunomodulatory molecule or cytokine is IL-15 or IL-21;
most preferably, the ROS-antagonizing domain is human GSTP1;
most preferably, the T2A has an amino acid sequence set forth in SEQ ID NO: 41;
most preferably, the T2A comprises a 2A element from thosea asigna virus (TaV);
most preferably, the T2A has a nucleotide sequence set forth in SEQ ID NO: 43;
most preferably, the human Ski has an amino acid sequence set forth in SEQ ID NO: 44;
most preferably, the human Ski has a nucleotide sequence set forth in SEQ ID NO: 45;
most preferably, the safety switch tEGFR is a truncated EGFR;
most preferably, the truncated EGFR is a truncated epidermal growth factor receptor;
most preferably, the tEGFR has an amino acid sequence set forth in SEQ ID NO: 48;
most preferably, the tEGFR has a nucleotide sequence set forth in SEQ ID NO: 49;
most preferably, the IL-15 has an amino acid sequence set forth in SEQ ID NO: 50;
most preferably, the IL-15 has a nucleotide sequence set forth in SEQ ID NO: 51;
most preferably, the IL-21 has an amino acid sequence set forth in SEQ ID NO: 52;
most preferably, the IL-21 has a nucleotide sequence set forth in SEQ ID NO: 53;
most preferably, the GSTP1 has an amino acid sequence set forth in SEQ ID NO: 54;
most preferably, the GSTP1 has a nucleotide sequence set forth in SEQ ID NO: 55;
most preferably, the chimeric antigen receptor is selected from any one of the group consisting of:
(1) a chimeric antigen receptor with an amino acid sequence set forth in SEQ ID NO: 56;
(2) a chimeric antigen receptor with an amino acid sequence set forth in SEQ ID NO: 58;
(3) a chimeric antigen receptor with an amino acid sequence set forth in SEQ ID NO: 60;
(4) a chimeric antigen receptor with an amino acid sequence set forth in SEQ ID NO: 62;
(5) a chimeric antigen receptor with an amino acid sequence set forth in SEQ ID NO: 64;
(6) a chimeric antigen receptor with an amino acid sequence set forth in SEQ ID NO: 66;
(7) a chimeric antigen receptor with an amino acid sequence set forth in SEQ ID NO: 68;
(8) a derived fusion protein formed by a substitution, deletion or addition of one or more amino acids to the amino acid sequence of the chimeric antigen receptor described in (1), (2), (3), (4), (5), (6), or (7).

Further, the chimeric antigen receptor described in the present application may further comprise one or more synthetic amino acids;
preferably, the synthetic amino acid includes (but is not limited to): aminocyclohexanecarboxylic acid, norleucine, α-amino n-decanoic acid, homoserine, S-acetamidomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N,N-dibenzyl-lysine, 6-hydroxylysine, ornithine, α-aminocyclopentanecarboxylic acid, α-aminocyclohexanecarboxylic acid, α-aminocycloheptanecarboxylic acid, α-(2-amino-2-norbornane)-carboxylic acid, α,γ-diaminobutyric acid, α,β-diaminopropionic acid, homophenylalanine, and α-tert-butylglycine.

The chimeric antigen receptor described in the present application can provide one or more of the following functions: targeting and disrupting B7H3-expressing cancer cells and/or tumor vasculature, reducing or eliminating cancer cells and/or tumor vasculature, promoting infiltration of immune cells into tumor sites and/or tumor vasculature, and enhancing/expanding anti-cancer and anti-tumor vasculature responses.

The chimeric antigen receptor described in the present application includes a functional variant of the chimeric antigen receptor described herein.

Further, the functional variant refers to a chimeric antigen receptor having substantial or significant sequence identity or similarity to a parent chimeric antigen receptor described herein. The functional variant retains the biological activity of the parent chimeric antigen receptor. The functional variant retains the ability to recognize target cells to a similar degree, to the same degree, or to a greater degree. The functional variant has about 92%, about 93%, about 94%, about 95%, about 96%, about 97%, about 98%, about 99% or more identity in sequence to the parent chimeric antigen receptor as compared to the parent chimeric antigen receptor.

Further, the functional variant may comprise an amino acid sequence of a parent chimeric antigen receptor having at least one conservative amino acid substitution. Alternatively or additionally, the functional variant may comprise an amino acid sequence of a parent chimeric antigen receptor having at least one non-conservative amino acid substitution. In such cases, it is preferred that the non-conservative amino acid substitution does not interfere with or inhibit the biological activity of the functional variant. The non-conservative amino acid substitution can enhance the biological activity of the functional variant, thereby increasing the biological activity of the functional variant as compared to the parent chimeric antigen receptor.

Further, the chimeric antigen receptor described in the present application may be glycosylated, amidated, carboxylated, phosphorylated, esterified, N-acylated, cyclized via, for example, a disulfide bond, or converted to an acid or an addition salt, and/or optionally dimerized or polymerized.

Further, the chimeric antigen receptor of the present application (including a functional variant) can be obtained by methods known in the art. Any suitable method for the preparation of polypeptides or proteins may be used to prepare them, for example by suitable de novo synthesis method for polypeptides and proteins. Likewise, polypeptides and proteins can be recombinantly produced using the nucleic acid described herein using a standard recombinant method. In addition, the chimeric antigen receptor of the present application can be isolated and/or purified from sources such as plants, bacteria, insects, and mammals. Methods for isolation and purification are well known in the art.

In a third aspect of the present application, provided is a polynucleotide.

Further, the polynucleotide has a sequence comprising: a nucleotide sequence encoding the HCVR of the antibody or the antigen-binding fragment thereof according to the first aspect of the present application, a nucleotide sequence encoding the LCVR of the antibody or the antigen-binding fragment thereof according to the first aspect of the present application, a nucleotide sequence encoding the antibody or the antigen-binding fragment thereof according to the first aspect of the present application or a nucleotide sequence encoding the chimeric antigen receptor according to the second aspect of the present application, or a complementary sequence thereof;
preferably, the nucleotide sequence encoding the chimeric antigen receptor according to the second aspect of the present application comprises a nucleotide sequence encoding a transmembrane domain, a nucleotide sequence encoding an intracellular signaling domain, a nucleotide sequence encoding a hinge region, a nucleotide sequence encoding a signal peptide, a nucleotide sequence encoding a co-stimulatory signaling domain, a nucleotide sequence encoding a self-cleaving peptide, a nucleotide sequence encoding a TGF-β-antagonizing domain, a nucleotide sequence encoding a safety switch, a nucleotide sequence encoding an immunomodulatory molecule or cytokine, and a nucleotide sequence encoding an ROS-inhibiting domain;
more preferably, the nucleotide sequence encoding the HCVR of the antibody or the antigen-binding fragment thereof according to the first aspect of the present application is set forth in SEQ ID NO: 9 or SEQ ID NO: 10;
more preferably, the nucleotide sequence encoding the LCVR of the antibody or the antigen-binding fragment thereof according to the first aspect of the present application is set forth in SEQ ID NO: 19 or SEQ ID NO: 20;
most preferably, the nucleotide sequence encoding the HCVR of the antibody or the antigen-binding fragment thereof according to the first aspect of the present application and the nucleotide sequence encoding the LCVR of the antibody or the antigen-binding fragment thereof according to the first aspect of the present application are linked by a Linker;
most preferably, a nucleotide sequence encoding the Linker is set forth in SEQ ID NO: 23 or SEQ ID NO: 24.

More preferably, the nucleotide sequence encoding the antibody or the antigen-binding fragment thereof according to the first aspect of the present application is set forth in SEQ ID NO: 27 or SEQ ID NO: 28;
more preferably, the nucleotide sequence encoding the transmembrane domain is set forth in SEQ ID NO: 30;
more preferably, the nucleotide sequence encoding the intracellular signaling domain is set forth in SEQ ID NO: 32;
more preferably, the nucleotide sequence encoding the hinge region is set forth in SEQ ID NO: 34;
more preferably, the nucleotide sequence encoding the signal peptide is set forth in SEQ ID NO: 36;
more preferably, the nucleotide sequence encoding the co-stimulatory signaling domain is set forth in SEQ ID NO: 38 or SEQ ID NO: 40;
more preferably, the nucleotide sequence encoding the self-cleaving peptide is set forth in SEQ ID NO: 43;
more preferably, the nucleotide sequence encoding the TGF-β-antagonizing domain is set forth in SEQ ID NO: 45;
more preferably, the nucleotide sequence encoding the safety switch is set forth in SEQ ID NO: 49;
more preferably, the nucleotide sequence encoding the immunomodulatory molecule or cytokine is set forth in SEQ ID NO: 51 or SEQ ID NO: 53;
more preferably, the nucleotide sequence encoding the ROS-inhibiting domain is set forth in SEQ ID NO: 55.

Most preferably, the nucleotide sequence encoding the chimeric antigen receptor according to the second aspect of the present application is set forth in SEQ ID NO: 57, SEQ ID NO: 59, SEQ ID NO: 61, SEQ ID NO: 63, SEQ ID NO: 65, SEQ ID NO: 67, or SEQ ID NO: 69.

Polynucleotides described in the present application, including oligonucleotides and nucleic acid molecules, generally refer to polymers of DNA or RNA, which may be single-stranded or double-stranded, may be synthetic or obtained, may comprise natural, non-natural or altered nucleotides, and may comprise natural, non-natural or altered internucleotide linkages, such as phosphoramidate linkages or phosphorothioate linkages. In some embodiments, the polynucleotide does not comprise any insertion, deletion, inversion and/or substitution. However, as discussed herein, in certain instances it may be appropriate for the polynucleotide to comprise one or more insertions, deletions, inversions and/or substitutions. In some embodiments, the polynucleotide can encode other amino acid sequences that do not affect the function of the polypeptide, protein, and chimeric antigen receptor and may or may not be translated by the host cell upon expression of the nucleic acid. In one embodiment of the present application, the polynucleotide is a complementary DNA (cDNA). In one embodiment of the present application, the polynucleotide comprises a codon-optimized nucleotide sequence.

In a fourth aspect of the present application, provided is a nucleic acid construct.

Further, the nucleic acid construct comprises the polynucleotide according to the third aspect of the present application;
preferably, the nucleic acid construct further comprises one or more regulatory sequences operably linked to the polynucleotide according to the third aspect of the present application and directing expression of the chimeric antigen receptor according to the second aspect of the present application in a host cell;
more preferably, the regulatory sequence comprises a sequence of a promoter, a sequence of a transcription terminator, and a sequence of a leader;
most preferably, the promoter comprises a CMV promoter, an EF-1α promoter, an SV40 early promoter, an MMTV promoter, a MoMuLV promoter, an avian leukemia virus promoter, an EB virus immediate early promoter, a Rous sarcoma virus promoter, an actin promoter, a myosin promoter, a heme promoter, a creatine kinase promoter, a metallothionein promoter, a glucocorticoid promoter, a progesterone promoter, and a tetracycline promoter;
most preferably, the transcription terminator comprises a CYC1 transcription terminator, a T7 transcription terminator, an rrnBT1 transcription terminator, an rrnBT2 transcription terminator, an ADH1 transcription terminator, a TIF51A transcription terminator, an ALG6 transcription terminator, an AOD transcription terminator, an AOX1 transcription terminator, an ARG4 transcription terminator, a PMA1 transcription terminator, a TEF1 transcription terminator, a TT1 transcription terminator, and a TT2 transcription terminator.

In a fifth aspect of the present application, provided is a recombinant vector.

Further, the recombinant vector comprises the polynucleotide according to the third aspect of the present application and the nucleic acid construct according to the fourth aspect of the present application;
preferably, the vector includes a cloning vector and an expression vector;
preferably, the vector includes a DNA vector, an RNA vector, a plasmid, and a virus-derived vector;
more preferably, the virus-derived vector includes a lentiviral vector, a retroviral vector, an adenoviral vector, an adeno-associated viral vector, a poxvirus vector, and a herpes virus vector.

Further, the retroviral vector includes, but is not limited to, the following mature commercial vectors: MSCV, MSCV-N WU ER, MSCV-N SM, MSCV IRES hCD4, mscv2.2, pMSCVII, pMSCVpuroATT, pMSCV_puro_41584, pMSCV_puro_41585, pMSCVII-LO, pMSCV_puro_41589, pMSCVII-AM, HOXA10-MSCV, HOXB4-NA-MSCV, HOXB6-NA-MSCV, HOXB6-WG-MSCV, HOXD4-WV-MSCV, PRRX2-MSCV, MEIS1B-MSCV, MSCV JMJD3, MSCV FLIP FF, MSCV P2Gm FF, pMSCV-FlagBcl10, MSCV-N GFP, and MSCV-C GFP.

Further, the vector described in the present application may be any suitable vector and may be used to transform or transfect any suitable host cell. Suitable vectors include vectors designed for propagation and amplification or expression, such as plasmids and viruses. The vector may be selected from pUC series, pBluescript series, pET series, pGEX series, and pEX series. Phage vectors such as λGT10, λGT11, AZapII (Stratagene), λEMBL4, and λNM1149 may also be used. Examples of plant vectors include pBI01, pBI101.2, pBI101.3, pBI121, and pBIN19 (Clontech). Examples of animal vectors include pEUK-C1, pMAM, and pMAMneo (Clontech).

In a sixth aspect of the present application, provided is an engineered host cell.

Further, the engineered host cell comprises the polynucleotide according to the third aspect of the present application, the nucleic acid construct according to the fourth aspect of the present application, and the recombinant vector according to the fifth aspect of the present application;
preferably, the host cell includes a eukaryotic cell and a prokaryotic cell;
more preferably, the host cell is a eukaryotic cell;
most preferably, the eukaryotic cell includes a mammalian cell, a plant cell, and a yeast cell;
most preferably, the eukaryotic cell is an immune cell;
most preferably, the immune cell includes a T cell, a B cell, an NK cell, an iNKT cell, a CTL cell, a dendritic cell, a myeloid cell, a monocyte and a macrophage, or any combination thereof;
most preferably, the immune cell is a T cell, an NK cell, or an iNKT cell.

In a seventh aspect of the present application, provided is an engineered host cell population.

Further, the engineered host cell population comprises the engineered host cells according to the sixth aspect of the present application;
preferably, the host cell population further comprises a host cell that does not comprise the polynucleotide according to the third aspect of the present application, the nucleic acid construct according to the fourth aspect of the present application, and the recombinant vector according to the fifth aspect of the present application;
more preferably, the host cell includes a prokaryotic cell and a eukaryotic cell;
most preferably, the prokaryotic cell includes a bacterium, an actinomycete, a cyanobacterium, mycoplasma, chlamydia, and rickettsia;
most preferably, the bacterium includes Escherichia coli, Bacillus subtilis, Salmonella typhimurium, Pseudomonas, Streptomyces, and Staphylococcus;
most preferably, the eukaryotic cell includes a mammalian cell, an insect cell, a plant cell, and a yeast cell;
most preferably, the host cell is an immune cell;
most preferably, the immune cell includes a T cell, a B cell, an NK cell, an iNKT cell, a CTL cell, a dendritic cell, a myeloid cell, a monocyte and a macrophage, or any combination thereof;
most preferably, the immune cell is a T cell, an NK cell, or an iNKT cell.

Further, the host cell can be obtained from a subject or a commercially available culture (e.g., American Type Culture Collection (ATCC));
preferably, the host cells can be obtained from a number of sources in the subject, including peripheral blood mononuclear cells, bone marrow, lymph node tissues, umbilical cord blood, thymus tissues, tissue from the site of infection, ascites, pleural effusion, spleen tissue, and tumors of the subject.

In an eighth aspect of the present application, provided is a derivative.

Further, the derivative comprises the antibody or the antigen-binding fragment thereof according to the first aspect of the present application and/or the chimeric antigen receptor according to the second aspect of the present application and/or the polynucleotide according to the third aspect of the present application with a detectable label, the antibody or the antigen-binding fragment thereof according to the first aspect of the present application and/or the chimeric antigen receptor according to the second aspect of the present application and/or the polynucleotide according to the third aspect of the present application conferring antibiotic resistance, and the antibody or the antigen-binding fragment thereof according to the first aspect of the present application and/or the chimeric antigen receptor according to the second aspect of the present application and/or the polynucleotide according to the third aspect of the present application bound or coupled to a therapeutic agent;
preferably, the detectable label includes a fluorescent dye, colloidal gold, a chemiluminescent label, and a chemiluminescent catalyst;
more preferably, the chemiluminescent label includes luminol and a derivative thereof, isoluminol and a derivative thereof, acridinium ester and a derivative thereof, adamantane, a rare earth element, and a bipyridine ruthenium complex;
more preferably, the chemiluminescent catalyst includes horseradish peroxidase and alkaline phosphatase;
preferably, a gene with the antibiotic resistance includes a penicillin resistance gene, a tetracycline resistance gene, a chloramphenicol resistance gene, and a kanamycin resistance gene;
preferably, the therapeutic agent includes a radionuclide, a cytokine, a gold nanoparticle, a viral particle, a liposome, a magnetic nanoparticle, a prodrug-activating enzyme, and a chemotherapeutic agent;
more preferably, the cytokine includes IL-2, IL-3, IL-4, IL-5, IL-6, IL-9, IL-10, IL-12, IL-13, IL-14, IFN-γ, TNF-β, TNF-α, G-CSF, and M-CSF;
more preferably, the chemotherapeutic agent includes cisplatin, paclitaxel, vincristine, asparaginase, oxaliplatin, platinum oxalate, and Eloxatin.

Further, the derivative described in the present application further comprises an immunoconjugate;
the immunoconjugate is formed by conjugating the antibody or the antigen-binding fragment thereof described in the present application to an effector molecule. The effector molecule may be any therapeutic molecule or marker molecule that facilitates detection. The effector molecule is not limited and may be any suitable effector molecule. For example, the effector molecule may be any one or more of a drug, a toxin, a label (e.g., any detectable label described herein), a small molecule, or another antibody or antigen-binding fragment thereof.

Further, the toxin may be pseudomonas exotoxin A or a variant thereof.

Further, examples of drugs that may be suitable for the immunoconjugate of the present application include (but are not limited to): pyrrolobenzodiazepine (PBD) dimers; tubulin binding agents such as dolastatin 10, monomethyl dolastatin 10, auristatin E, monomethyl auristatin E (MMAE), auristatin F, monomethyl auristatin F, HTI-286, tubulysin M, maytansinoid AP-3, cryptophycin, Boc-Val-Dil-Dap-OH, tubulysin IM-1, Boc-Val-Dil-Dap-Phe-OMe, tubulysin IM-2, Boc-Nme-Val-Val-Dil-Dap-OH, tubulysin IM-3, and colchicine DA; DNA alkylating agents (duocarmycin analogs) such as duocarmycin SA, duocarmycin CN, duocarmycin DMG, duocarmycin DMA, duocarmycin MA, duocarmycin TM, duocarmycin MB, and duocarmycin GA; tomaymycin DM; SJG-136; illudin S; irofulven, apaziquone, triptolide, staurosporine, camptothecin, methotrexate; and other anti-cancer drugs, such as kinase inhibitors, histone deacetylase (HDAC) inhibitors, proteasome inhibitors, and matrix metalloproteinase (MMP) inhibitors.

Further, marker molecules that may be suitable for the immunoconjugate of the present application are, for example, a radioisotope, a fluorophore (e.g., fluorescein isothiocyanate (FITC) and phycoerythrin (PE)), an enzyme (e.g., alkaline phosphatase and horseradish peroxidase), and an elemental particle (e.g., a gold particle).

In a ninth aspect of the present application, provided is a pharmaceutical composition.

Further, the pharmaceutical composition comprises the antibody or the antigen-binding fragment thereof according to the first aspect of the present application and/or the chimeric antigen receptor according to the second aspect of the present application and/or the polynucleotide according to the third aspect of the present application, the nucleic acid construct according to the fourth aspect of the present application, the recombinant vector according to the fifth aspect of the present application, the engineered host cell according to the sixth aspect of the present application, the engineered host cell population according to the seventh aspect of the present application, or the derivative according to the eighth aspect of the present application;
preferably, the pharmaceutical composition further comprises one or more pharmaceutically or physiologically acceptable carrier, diluent or excipient combinations.

Further, such combinations may include: a buffer, such as neutral buffered saline and phosphate buffered saline; a carbohydrate, such as glucose, mannose, sucrose, dextran, or mannitol; a protein; a polypeptide or an amino acid, such as glycine; an antioxidant; a chelating agent, such as EDTA or glutathione; an adjuvant, such as aluminum hydroxide; and a preservative.

Further, suitable pharmaceutically acceptable carriers, diluents or excipients are described in detail in Remington's Pharmaceutical Sciences (19th ed., 1995). The substances are to assist the stability of the formulation or to improve the activity or its bioavailability or to give an acceptable taste or smell in case of oral administration, as required. The formulations which may be used in such pharmaceutical compositions may be in the form of their original compounds themselves, or optionally in the form of their pharmaceutically acceptable salts. The pharmaceutical composition thus formulated may be administered by any suitable means known to those skilled in the art, as required, and a safe and effective amount of the drug of the present application is administered to a human when the pharmaceutical composition is used.

The appropriate dose of the pharmaceutical composition of the present application may be prescribed in various ways depending on factors such as the method of preparation; the mode of administration; and the age, body weight, sex, disease state, diet, administration time, administration route, excretion rate and reaction sensitivity of the patient. A skilled physician can usually determine the prescription and the dose of administration effective for the desired treatment or prevention with ease.

The pharmaceutical composition disclosed in the present application can be formulated for oral, intravenous, topical, enteral and/or parenteral administration as required.

In a tenth aspect of the present application, provided is a kit.

Further, the kit comprises the chimeric antigen receptor according to the second aspect of the present application, the polynucleotide according to the third aspect of the present application, the nucleic acid construct according to the fourth aspect of the present application, or the recombinant vector according to the fifth aspect of the present application;
preferably, the kit further comprises a reagent for introducing the chimeric antigen receptor, the polynucleotide, the nucleic acid construct, or the recombinant vector into a host cell;
preferably, the kit further comprises instructions for introducing the chimeric antigen receptor, the polynucleotide, the nucleic acid construct, or the recombinant vector into a host cell.

In an eleventh aspect of the present application, provided is a biological agent comprising the engineered host cell according to the sixth aspect of the present application or the engineered host cell population according to the seventh aspect of the present application.

Further, the biological agent may be used in combination with other therapeutic drugs.

In a twelfth aspect of the present application, provided is any one of the following methods:
(1) a method for stimulating an immune response to a target cell population or tissue in a mammal;
further, the method comprises the following steps: administering an effective amount of the engineered host cell according to the sixth aspect of the present application, the engineered host cell population according to the seventh aspect of the present application, the derivative according to the eighth aspect of the present application, the pharmaceutical composition according to the ninth aspect of the present application, or the biological agent according to the eleventh aspect of the present application to the mammal;
(2) a method for preparing the engineered host cell according to the sixth aspect of the present application or the engineered host cell population according to the seventh aspect of the present application;
further, the method comprises the following steps: introducing the polynucleotide according to the third aspect of the present application, the nucleic acid construct according to the fourth aspect of the present application, or the recombinant vector according to the fifth aspect of the present application into the host cell;
preferably, the introduction is conducted by means including physical means, chemical means, and biological means;
more preferably, the physical means includes calcium phosphate precipitation, lipofection, particle bombardment, microinjection, and electroporation;
more preferably, the chemical means includes a colloidal dispersion system and a lipid-based system;
most preferably, the colloidal dispersion system includes a macromolecular complex, a nanocapsule, a microsphere, and a bead;
most preferably, the lipid-based system includes an oil-in-water emulsion, a micelle, a mixed micelle, and a liposome;
more preferably, the biological means includes a DNA vector, an RNA vector, a lentiviral vector, a poxvirus vector, a herpes simplex virus vector, an adenoviral vector, and an adeno-associated viral vector.

Further, the introduction means described in the present application may introduce the nucleic acid molecule or vector as described above into a cell by various suitable means, and are not limited to the methods listed in the present application, such as calcium phosphate transfection, DEAE-dextran-mediated transfection, microinjection, electroporation, TALEN method, ZFN method, non-viral vector (e.g., liposome)-mediated transfection or viral vector-mediated transfection (e.g., lentivirus infection, retrovirus infection, and adenovirus infection), and other physical, chemical or biological means for transferring into a cell, such as transposon technology and CRISPR-Cas9.
(3) a method for regulating an immune response in a subject;

Further, the method comprises the following steps: administering the engineered host cell according to the sixth aspect of the present application, the engineered host cell population according to the seventh aspect of the present application, the derivative according to the eighth aspect of the present application, the pharmaceutical composition according to the ninth aspect of the present application, or the biological agent according to the eleventh aspect of the present application to the subject;
(4) a method for selecting a medicament candidate for preventing and/or treating a tumor; further, the method comprises the following steps:
(I) providing a test substance and a positive control substance, wherein the positive control substance is the engineered host cell according to the sixth aspect of the present application and/or the engineered host cell population according to the seventh aspect of the present application; and
(II) detecting, in a test group, a killing effect of the test substance in step (I) on tumor cells, and comparing the killing effect with corresponding experimental results in a positive control group and a negative control group;
preferably, in step (II), the test results in the test group are compared with the test results in the positive control group and the negative control group, and if the killing effect on the tumor cells in the test group is significantly lower than that in the negative control group, and the killing effect of the test substance on the tumor cells in the test group (A1)/the killing effect of the engineered host cell according to the sixth aspect of the present application and/or the engineered host cell population according to the seventh aspect of the present application on the tumor cells in the positive control group (A2) is greater than or equal to 80%, then it is suggested that the test substance is a medicament candidate for preventing and/or treating the tumor;
(5) a method for producing the antibody or the antigen-binding fragment thereof according to the first aspect of the present application;
further, the method comprises the following steps: culturing the engineered host cell according to the sixth aspect of the present application and/or the engineered host cell population according to the seventh aspect of the present application, and isolating the antibody or the antigen-binding fragment thereof according to the first aspect of the present application from the culture;
(6) a method for detecting B7H3 in a test sample;
further, the method comprises the following steps: contacting the test sample with the antibody or the antigen-binding fragment thereof according to the first aspect of the present application, and detecting formation of a complex by the antibody or the antigen-binding fragment thereof and B7H3;
preferably, the antibody or the antigen-binding fragment thereof is labeled with a detectable label;
more preferably, the detectable label includes a fluorochrome, an avidin, a paramagnetic atom, and a radioisotope;
most preferably, the fluorochrome is fluorescein, rhodamine, Texas red, phycoerythrin, phycocyanin, allophycocyanin, or peridinin-chlorophyll protein;
most preferably, the avidin is biotin, egg white avidin, streptavidin, egg yolk avidin, or an avidin analog;
most preferably, the radioisotope is radioactive iodine, radioactive cesium, radioactive iridium, or radioactive cobalt;
(7) a method for specifically inhibiting the activity of B7H3;
further, the method comprises the following steps: introducing the polynucleotide according to the third aspect of the present application into a cell of an organism, and inhibiting the activity of B7H3 by expressing the antibody or the antigen-binding fragment thereof according to the first aspect of the present application;
(8) a method of treatment;
further, the method of treatment comprises administering the antibody or the antigen-binding fragment thereof according to the first aspect of the present application, the chimeric antigen receptor according to the second aspect of the present application, the polynucleotide according to the third aspect of the present application, the nucleic acid construct according to the fourth aspect of the present application, the recombinant vector according to the fifth aspect of the present application, the engineered host cell according to the sixth aspect of the present application, the engineered host cell population according to the seventh aspect of the present application, the derivative according to the eighth aspect of the present application, the pharmaceutical composition according to the ninth aspect of the present application, or the biological agent according to the eleventh aspect of the present application to a subject with a disease or disorder associated with B7H3;
preferably, the disease or disorder associated with B7H3 includes a tumor expressing B7H3;
more preferably, the tumor includes ovarian cancer, kidney cancer, lung cancer, breast cancer, colorectal cancer, esophageal cancer, prostate cancer, oral cancer, gastric cancer, pancreatic cancer, endometrial cancer, liver cancer, bladder cancer, osteosarcoma, glioma, acute myeloid leukemia, non-Hodgkin lymphoma, Hodgkin lymphoma, brain cancer, cervical cancer, head and neck cancer, testicular cancer, pituitary cancer, esophagus cancer, skin cancer, bone cancer, B-cell lymphoma, T-cell lymphoma, myeloma, hematopoietic tumor, thymoma, anal cancer, primary or metastatic melanoma, squamous cell cancer, basal cell carcinoma, angiosarcoma, hemangioendothelioma, thyroidcancer, soft tissue sarcoma, gastrointestinal cancer, intrahepatic cholangiocarcinoma, joint cancer, nasal cancer, and any other cancer now known or later discovered (see, e.g., Rosenberg (1996) Ann. Med. 47: 481-491, the entire content of which is incorporated herein by reference).

Further, the subject includes (but is not limited to): human and non-human animals, wherein the non-human animal includes rabbit, rat, mouse, monkey or other lower primates.

In a thirteenth aspect of the present application, provided is use of any one of the following aspects:
(1) use of the antibody or the antigen-binding fragment thereof according to the first aspect of the present application, the chimeric antigen receptor according to the second aspect of the present application, the polynucleotide according to the third aspect of the present application, the nucleic acid construct according to the fourth aspect of the present application, the recombinant vector according to the fifth aspect of the present application, the engineered host cell according to the sixth aspect of the present application, the engineered host cell population according to the seventh aspect of the present application, the derivative according to the eighth aspect of the present application, the pharmaceutical composition according to the ninth aspect of the present application, or the biological agent according to the eleventh aspect of the present application in the preparation of a medicament for preventing and/or treating a tumor;
(2) use of the antibody or the antigen-binding fragment thereof according to the first aspect of the present application, the chimeric antigen receptor according to the second aspect of the present application, the polynucleotide according to the third aspect of the present application, the nucleic acid construct according to the fourth aspect of the present application, the recombinant vector according to the fifth aspect of the present application, the engineered host cell according to the sixth aspect of the present application, the engineered host cell population according to the seventh aspect of the present application, or the derivative according to the eighth aspect of the present application in the preparation of a kit for preparing an immune cell for preventing and/or treating a tumor;
(3) use of the antibody or the antigen-binding fragment thereof according to the first aspect of the present application, the chimeric antigen receptor according to the second aspect of the present application, the polynucleotide according to the third aspect of the present application, the nucleic acid construct according to the fourth aspect of the present application, the recombinant vector according to the fifth aspect of the present application, the engineered host cell according to the sixth aspect of the present application, the engineered host cell population according to the seventh aspect of the present application, the derivative according to the eighth aspect of the present application, the pharmaceutical composition according to the ninth aspect of the present application, the kit according to the tenth aspect of the present application, or the biological agent according to the eleventh aspect of the present application in the preparation of a biological agent for preventing and/or treating a tumor;
(4) use of the pharmaceutical composition according to the ninth aspect of the present application in the prevention and/or treatment of a tumor;
(5) use of the kit according to the tenth aspect of the present application in the preparation of an immune cell for preventing and/or treating a tumor;
(6) use of the biological agent according to the eleventh aspect of the present application in the prevention and/or treatment of a tumor;
(7) use of the chimeric antigen receptor according to the second aspect of the present application in the preparation of a polynucleotide, a nucleic acid construct, a recombinant vector, an engineered host cell, an engineered host cell population, or a derivative;
(8) use of the polynucleotide according to the third aspect of the present application in the preparation of a nucleic acid construct, a recombinant vector, an engineered host cell, an engineered host cell population, or a derivative;
(9) use of the nucleic acid construct according to the fourth aspect of the present application in the preparation of a recombinant vector, an engineered host cell, an engineered host cell population, or a derivative;
(10) use of the recombinant vector according to the fifth aspect of the present application in the preparation of an engineered host cell, an engineered host cell population, or a derivative;
(10) use of the engineered host cell according to the sixth aspect of the present application in the preparation of an engineered host cell population or a derivative;
(11) use of the engineered host cell population according to the seventh aspect of the present application in the preparation of a derivative;
preferably, the tumor includes a tumor expressing B7H3;
more preferably, the tumor includes ovarian cancer, kidney cancer, lung cancer, breast cancer, colorectal cancer, esophageal cancer, prostate cancer, oral cancer, gastric cancer, pancreatic cancer, endometrial cancer, liver cancer, bladder cancer, osteosarcoma, glioma, acute myeloid leukemia, non-Hodgkin lymphoma, Hodgkin lymphoma, brain cancer, cervical cancer, head and neck cancer, testicular cancer, pituitary cancer, esophagus cancer, skin cancer, bone cancer, B-cell lymphoma, T-cell lymphoma, myeloid leukemia, myeloma, hematopoietic tumor, thymoma, anal cancer, primary or metastatic melanoma, squamous cell cancer, basal cell carcinoma, angiosarcoma, hemangioendothelioma, thyroidcancer, soft tissue sarcoma, gastrointestinal cancer, intrahepatic cholangiocarcinoma, joint cancer, nasal cancer, and any other cancer now known or later discovered (see, e.g., Rosenberg (1996) Ann. Med. 47: 481-491, the entire content of which is incorporated herein by reference).

The chimeric antigen receptor described in the present application includes (but is not limited to): a chimeric antigen receptor with an amino acid sequence set forth in SEQ ID NO: 56, a chimeric antigen receptor with an amino acid sequence set forth in SEQ ID NO: 58, a chimeric antigen receptor with an amino acid sequence set forth in SEQ ID NO: 60, a chimeric antigen receptor with an amino acid sequence set forth in SEQ ID NO: 62, a chimeric antigen receptor with an amino acid sequence set forth in SEQ ID NO: 64, a chimeric antigen receptor with an amino acid sequence set forth in SEQ ID NO: 66, a chimeric antigen receptor with an amino acid sequence set forth in SEQ ID NO: 68, and a derived fusion protein formed by a substitution, deletion or addition of one or more amino acids to the amino acid sequence of the chimeric antigen receptor described in SEQ ID NO: 56, SEQ ID NO: 58, SEQ ID NO: 60, SEQ ID NO: 62, SEQ ID NO: 64, SEQ ID NO: 66, or SEQ ID NO: 68. In addition, chimeric antigen receptors corresponding to amino acid sequences of SEQ ID NO: 56, SEQ ID NO: 58, SEQ ID NO: 60, SEQ ID NO: 62, SEQ ID NO: 64, SEQ ID NO: 66, and SEQ ID NO: 68 that do not comprise signal peptides, self-cleaving peptides, TGF-β-antagonizing domains, safety switches, immunomodulatory molecules or cytokines, and/or ROS-inhibiting domains are also included within the protection scope of the present application.

In order to better understand the technical solutions of the present application, terms involved in the present application are explained as follows, and the following terms involved in the present application refer to the following contents unless otherwise specified.

The term “B7H3” as used herein is synonymous with “CD276”, which belongs to the B7 immune checkpoint superfamily and is a type I transmembrane protein composed of an extracellular domain comprising two pairs of identical immunoglobulin variable and constant regions, and a shorter intracellular domain.

The term “expression vector” as used herein refers to a vector comprising a recombinant polynucleotide comprising an expression control sequence operably linked to a nucleotide sequence to be expressed. Expression vectors comprise sufficient cis-regulatory elements for expression, and other elements for expression can be provided by a host cell or in an in vitro expression system. Expression vectors include all those known in the art that are incorporated into recombinant polynucleotides, such as plasmids (e.g., naked or contained in liposomes) and viruses (e.g., Sendai virus, lentiviruses, retroviruses, adenoviruses, and adeno-associated viruses).

The term “cloning vector” as used herein refers to a DNA molecule such as a plasmid, a cosmid, or a phage that has the ability to replicate autonomously in a host cell. Cloning vectors typically comprise one or a small number of restriction endonuclease recognition sites at which foreign DNA sequences are inserted in a defined manner without loss of the essential biological function of the vector, as well as marker genes suitable for use in the identification and selection of cells transformed with the cloning vector. Marker genes typically include genes that provide tetracycline resistance or ampicillin resistance.

The present application has the following advantages and beneficial effects:

The present application provides a novel fully human anti-human B7H3 antibody, a fully human B7H3-targeting chimeric antigen receptor comprising the antibody, a genetically engineered cell expressing the receptor and the antibody and use thereof in adoptive cell therapy based on the high expression characteristic of B7H3 in tumor cells and the characteristic of the inhibitory activity of B7H3 on immune cells. Experiments have proven that the fully human anti-human B7H3 antibody provided by the present application has high sensitivity of B7H3 detection, high affinity for B7H3, and strong specificity, and lays a foundation for development of anti-tumor drugs, anti-tumor treatment, studies on tumor mechanisms and the like; experiments have proven that cells of the B7H3-targeting CAR-T (B7H3-02) with high expression of hSki, the CAR-iNKT (B7H3-02) comprising IL-15, the CAR-iNKT (B7H3-02) comprising IL-21, the CAR-NK (B7H3-02) comprising IL-15, the CAR-T (B7H3-01), the CAR-T (B7H3-02), and the CAR-T (B7H3-02) with high expression of GSTP1 prepared by the present application have strong proliferation capacity, cytokine release capacity, and killing capacity of various solid tumor cells, are high in killing activity, safe and effective, and can effectively eliminate tumor cells, thus having important application prospects in the field of tumor cell immunotherapy.

### BRIEF DESCRIPTION OF THE DRAWINGS

Embodiments of the present application are described in detail below with reference to the accompanying drawings.

FIG. 1 shows a statistical graph of the enrichment results of phage clones for screening specific binding antibodies;

FIG. 2 shows the results of detection of the chromogenic reaction of B7H3-02 monoclonal phage recognizing and binding to B7H3 target antigen by ELISA;

FIG. 3 shows the statistical graph of detection of the chromogenic reaction data of B7H3-02 monoclonal phage recognizing and binding to B7H3 target antigen by ELISA;

FIG. 4 shows the identification results of PCR amplification of B7H3-02 scFv and its prokaryotic expression vector by DNA electrophoresis detection, wherein FIG. 4 panel A: B7H3-02 scFv, FIG. 4 panel B: pET22b-B7H3-02 scFv;

FIG. 5 shows the results of purification of B7H3-02 scFv protein by prokaryotic expression;

FIG. 6 shows the results of detection of the chromogenic reaction for the ability of the purified B7H3-02 scFv protein to recognize the B7H3 target antigen by ELISA;

FIG. 7 shows the statistical graph of detection of the chromogenic reaction data for the ability of the purified B7H3-02 scFv protein to recognize the B7H3 target antigen by ELISA;

FIG. 8 shows the results of detection of binding constant and dissociation constant for the purified B7H3-02 antibody and B7H3 target antigen by Biacore, wherein FIG. 8 panel A: results, FIG. 8 panel B: result statistical graph;

FIG. 9 shows the results of detection of the ability of the B7H3-02 scFv expressed on the surface of eukaryotic cells to bind to the B7H3 target antigen by flow cytometry and a statistical graph of mean fluorescence intensity, wherein FIG. 9 panel A: flow cytometry detection results, FIG. 9 panel B: statistical graph of mean fluorescence intensity;

FIG. 10 shows the results of detection of the chromogenic reaction of clones CD276-01 and CD276-03 recognizing and binding to CD276 target antigen by ELISA;

FIG. 11 shows the results of detection of the chromogenic reaction for the ability of the purified scFv protein to recognize a target antigen by ELISA;

FIG. 12 shows the results of purification of scFv protein;

FIG. 13 shows the results of analysis of the ability of scFv to recognize and bind to a target antigen by flow cytometry;

FIG. 14 shows the results of validation of the prepared CAR-T cells, wherein FIG. 14 panel A: results of detection of CAR expression in CAR-T by flow cytometer, FIG. 14 panel B: statistical graph of results of detection of CAR expression in CAR-T by flow cytometer, FIG. 14. Panel C: CAR-T cell growth curve, and FIG. 14 panel D: results of detection of hSki expression in CAR-T cells by Western blot;

FIG. 15 shows the results of the effect of TGF-β on the ability of CAR-T cells to kill tumor cells;

FIG. 16 shows the results of secretion of IFN-γ by CAR-T cells with high expression of hSki;

FIG. 17 shows the results of the ability of CAR-T with high expression of hSki prepared by the present application to clear mouse lung cancer transplanted tumors, wherein panel A: experimental flow chart, panel B: results of tumor volume in mice on different days, and C: statistical graph of the results of tumor volume in mice on day 51 after formation of a tumor by the tumor cell injected;

FIG. 18 shows the results of detection of CAR expression in B7H3-CAR-iNKT by flow cytometer;

FIG. 19 shows the statistical graph of the results of detection of CAR expression in B7H3-CAR-iNKT by flow cytometer;

FIG. 20 shows the growth curve of B7H3-CAR-iNKT cells;

FIG. 21 shows the results of detection of the proliferation capacity of B7H3-CAR-iNKT cells in different kidney cancer cells, wherein FIG. 21 panel A: 786-O, FIG. 21 panel B: OSRC-2;

FIG. 22 shows the results of detection of the cytokine release capacity of B7H3-CAR-iNKT cells in different kidney cancer cells, wherein FIG. 22 panel A: IFN-γ, FIG. 22 panel B: IL-2;

FIG. 23 shows the results of the killing ability of B7H3-CAR-iNKT on kidney cancer cells, wherein FIG. 23 panel A: 786-O, FIG. 23 panel B: OSRC-2;

FIG. 24 shows the results of the ability of B7H3-CAR-iNKT to clear mouse kidney cancer transplanted tumor, wherein panel A: experimental procedure, panel B: tumor volume, panel C: statistical graph of B7H3-CAR-iNKT cell number in blood, and panel D: survival curve;

FIG. 25 shows the results of the ability of B7H3-CAR-iNKT to kill ovarian cancer cells SKOV-3 in vitro;

FIG. 26 shows the results of the ability of B7H3-CAR-iNKT to clear mouse ovarian cancer peritoneal transplanted tumors, wherein FIG. 26 panel A: experimental flow chart, FIG. 26 panel B: fluorescence imaging of mice, FIG. 26 panel C: statistical graph of relative luminous intensity, and FIG. 26 panel D: statistical graph of B7H3-CAR-iNKT cell number in blood;

FIG. 27 shows the results of detection of CAR positive rate by flow cytometer, wherein, FIG. 27 panel A: UT-INKT, FIG. 27 panel B: B7H3.CAR-iNKT, and FIG. 27 panel C: B7H3.CAR/IL-21-iNKT;

FIG. 28 shows the statistical graph of the results of detection of CAR transduction rate by flow cytometer;

FIG. 29 shows the results of detection of the cytokine release capacity of B7H3.CAR/IL-21-iNKT cells when co-cultured with different kidney cancer cells, wherein FIG. 29 panel A: IFN-γ, FIG. 29 panel B: IL-2;

FIG. 30 shows the results of detection of apoptosis of B7H3.CAR/IL-21-iNKT cells by flow cytometer;

FIG. 31 shows the results of detection of the killing effect of B7H3.CAR/IL-21-iNKT cells on kidney cancer cells 786-O by RTCA experiment, wherein panel A: E/T=2/1, panel B: E/T=1/1, and panel C: E/T=½;

FIG. 32 shows the results of detection of the killing effect of B7H3.CAR/IL-21-iNKT cells on kidney cancer cells OSRC-2 by RTCA experiment, wherein panel A: E/T=5/1, panel B: E/T=1/1, and panel C: E/T=⅕;

FIG. 33 shows the results of the ability of B7H3.CAR/IL-21-iNKT to clear mouse kidney cancer subcutaneously transplanted tumor, wherein FIG. 33 panel A: experimental flow chart, FIG. 33 panel B: statistical graph of mouse tumor volume, and FIG. 33 panel C: statistical graph of CAR-iNKT cell number in peripheral blood;

FIG. 34 is a flow cytometric representative plot of purity assay for NK cells cultured for different days, wherein panels A, B, C, and D are assay graphs on day 0, day 7, day 10, and day 14, respectively, with fluorescence intensity of Alexa Fluor488 as the abscissa and fluorescence intensity of APC as the ordinate; FIG. 34 panel E is also the assay graph on day 14, with fluorescence intensity of APC as the abscissa and fluorescence intensity of PerCP/Cy5.5 as the ordinate;

FIG. 35 is a flow cytometric representative plot of CAR-NK transfection efficiency assay, wherein the left image is blank control, the middle image is NK cells expressing CAR that does not comprise IL-15, and the right image is NK cells comprising CAR of IL-15;

FIG. 36 is a statistical graph of CAR-NK transfection efficiency;

FIG. 37 is a dynamic curve of the killing of the CAR-NK cell on breast cancer cell MCF-7 analyzed by RTCA technology, wherein the upper image is NK cells expressing CAR that does not comprise IL-15, and the lower image is NK cells comprising CAR of IL-15;

FIG. 38 shows the results of detection of CD276-CAR expression in CAR-T by flow cytometer, wherein panel A: control; panel B: CD276-CAR;

FIG. 39 shows the growth curve of CAR-T cells;

FIG. 40 shows the results of the killing ability of CAR-T of the present application on SKOV3 cells, wherein panel A: 2:1; panel B: 1:1; panel C: 1:2; the ordinate of the graph is the normalized cell index and the abscissa of the graph is time (h);

FIG. 41 shows the results of the killing ability of CAR-T of the present application on A549 cells, wherein panel A: 2:1; panel B: 1:1; panel C: 1:2; the ordinate of the graph is the normalized cell index and the abscissa of the graph is time (h);

FIG. 42 shows the results of detection of CD276-CAR expression in CAR-T by flow cytometer;

FIG. 43 shows the growth curve of CAR-T cells;

FIG. 44 shows the results of the killing ability of CAR-T of the present application on SKOV3 cells, wherein panel A: 2:1; panel B: 1:1; panel C: 1:2;

FIG. 45 shows the results of the killing ability of CAR-T of the present application on A549 cells, wherein panel A: 2:1; panel B: 1:1; panel C: 1:2;

FIG. 46 shows the results of the ability of CAR-T of the present application to clear mouse ovarian cancer transplanted tumors, wherein panel A: experimental flow chart; panel B: fluorescence imaging of mice; panel C: statistical graph;

FIG. 47 shows the results of validation of the prepared CAR-T cells, wherein FIG. 47 panel A: results of detection of CAR expression in CAR-T by flow cytometer, FIG. 47 panel B: statistical graph of results of detection of CAR expression in CAR-T by flow cytometer, FIG. 47 panel C: CAR-T cell growth curve, and FIG. 47 panel D: results of detection of hGSTP1 expression in CAR-T cells by Western blot;

FIG. 48 shows the results of the effect of high expression of hGSTP1 on the level of reactive oxygen species of CAR-T cells, wherein panel A: flow cytometry graph; panel B: statistical graph;

FIG. 49 shows the results of the effect of the tumor killing function of CAR-T cells with high expression of hGSTP1, wherein panel A: flow cytometry graph; panel B: statistical graph;

FIG. 50 shows the results of the ability of CAR-T with high expression of hGSTP1 prepared by the present application to clear mouse lung cancer transplanted tumors, wherein panel A: experimental flow chart; panel B: results of tumor volume in mice on different days; panel C: statistical graph of the results of tumor volume in mice on day 51 after formation of a tumor by the tumor cell injected.

### DETAILED DESCRIPTION

The present application will be further illustrated with reference to the following specific examples, which are illustrative only and are not to be construed as limiting the present application. It can be understood by those of ordinary skill in the art that various changes, modifications, replacements and variations can be made to these examples without departing from the principle and purpose of the present application, and the scope of the present application is defined by the claims and equivalents thereof. Experimental procedures without specified conditions in the following examples, are generally carried out under conventional conditions, or under conditions recommended by the manufacturer.

### Example 1. Screening for the Fully Human B7H3 Single-Chain Antibody (B7H3 scFv) (B7H3-02)

### 1. Experimental Condition Setup

An experimental group, a control group 1, and a control group 2 were separately set, wherein the experimental conditions of the groups were respectively as follows:
Experimental group: B7H3 antigen+B7H3-Phage
Control group 1: other biotin-free antigen (PRPS1)+B7H3-Phage
Control group 2: no antigen+B7H3-Phage

### 2. Method

After four rounds of screenings, specific binding antibody sequences were enriched, wherein the total amount of phage input, the quantity of antigens added, and the reaction time, among other conditions, were varied in each round;
The final results were obtained by counting the number of infectious phages contained in the eluate of 0.1 M HCl (PH=2.0) per 100 μL of the control group and the experimental group, and the enrichment was determined.

### 3. Results

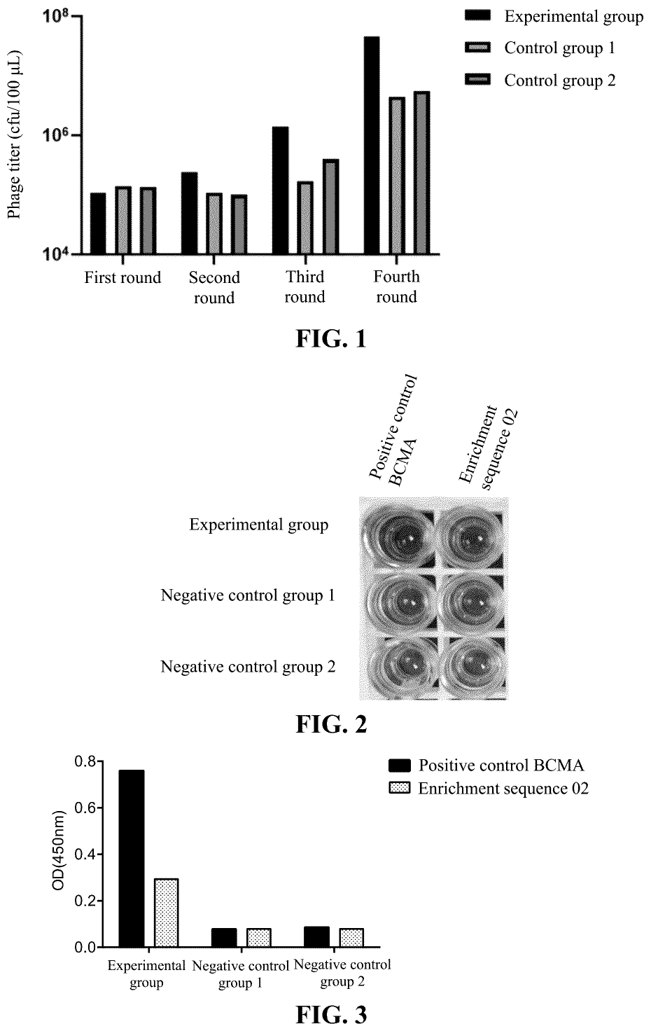

(1) analysis of screening results: the experimental results are respectively shown in Table 1 and FIG. 1, and the results show that enrichment occurred in the third round of screening, and the ratio of the number of the phages (specific binding of antigen and antibody) eluted in the experimental group to the control group (non-specific binding between antigen and antibody, or no affinity) was close to 10 times; after the experimental conditions were changed in the fourth round, the experimental group and the control group still kept 10-fold difference, indicating that the screened phage has scFv with affinity for the B7H3 target protein;
(2) scFv sequence analysis: 24 monoclones were picked for sequencing, 13 of which expressed scFv sequences in their entirety, and the sequences were enriched:
clone 02 (clone B7H3-02): VH: IGHV3-23*01/IGHV3-23D*01, IGHJ4*02/IGHJ4*0303; VK: IGKV1-39*01/IGKVID-39*01, IKJ1*01;
clone 03 (clone B7H3-02): VH: IGHV3-33*06, IGHJ6*03; VL: IGKV2-14*01, IGLJ2*01/IGLJ3*01;
the scFv amino acid sequence of clone 02 is set forth in SEQ ID NO: 25 by sequencing;
the amino acid sequence of scFv of clone 03 is set forth in SEQ ID NO: 70, and the nucleotide sequence is set forth in SEQ ID NO: 71 by sequencing;
the amino acid sequence of HCDR1 of the heavy chain variable region (HCVR) of clone 02 is set forth in SEQ ID NO: 1, the amino acid sequence of HCDR2 is set forth in SEQ ID NO: 3, the amino acid sequence of HCDR3 is set forth in SEQ ID NO: 5, the amino acid sequence of HCVR is set forth in SEQ ID NO: 7, and the nucleotide sequence of HCVR is set forth in SEQ ID NO: 9; the amino acid sequence of LCDR1 of the light chain variable region (LCVR) of clone 02 is set forth in SEQ ID NO: 11, the amino acid sequence of LCDR2 is set forth in SEQ ID NO: 13, the amino acid sequence of LCDR3 is set forth in SEQ ID NO: 15, the amino acid sequence of LCVR is set forth in SEQ ID NO: 17, the nucleotide sequence of LCVR is set forth in SEQ ID NO: 19, the amino acid sequence of the linker of clone 02 is set forth in SEQ ID NO: 21, the nucleotide sequence is set forth in SEQ ID NO: 23, and the nucleotide sequence of clone 02 is set forth in SEQ ID NO: 27.

TABLE 1
Screening results

| | First round | Second round | Third round | Fourth round |
|---|---|---|---|---|
| | Condition setup | | | |
| | 3 experimental | 4 experimental | 3 experimental | 3 experimental |
| | wells | wells | wells | wells |
| | The total input | The total input | The total input | The total input |
| | amount of | amount of | amount of | amount of |
| | phages: 3.6 × 10¹¹ | phages: 1.4 × 10¹¹ | phages: 1.2 × 10¹¹ | phages: 8.3 × 10¹⁰ |
| | Antigen amount | Antigen amount | Antigen amount | Antigen amount |
| | 1.5 μL/well | 1.5 μL/well | 1.5 μL/well | 1.5 μL/well |
| | Incubation | Incubation | Incubation | Incubation |
| | time 1 h | time 1 h | time 1 h | time 1 h |
| | Result | | | |
| | PH = 2.0 elution | PH = 2.0 elution | PH = 2.0 elution | PH = 2.0 elution |
| Phage concentration | 1.2 × 10⁹ | 3.74 × 10⁸ | 1.2 × 10⁹ | 3.1 × 10⁸ |
| (AMP)/100 μL | | | | |
| Amount of phages | 1.1 × 10⁵ | 2.4 × 10⁵ | 1.385 × 10⁶ | 4.62 × 10⁷ |
| eluted in the | | | | |
| experimental | | | | |
| group/100 μL | | | | |
| Amount of phages | 1.41 × 10⁵ | 1.08 × 10⁵ | 1.69 × 10⁵ | 4.48 × 10⁶ |
| eluted in the control | | | | |
| group 1/100 μL | | | | |
| Amount of phages | 1.38 × 10⁵ | 1 × 10⁵ | 4 × 10⁵ | 5.6 × 10⁶ |
| eluted in the control | | | | |
| group 2/100 μL | | | | |

### Example 2. Detection of the Ability of the Fully Human B7H3 scFv (B7H3-02) to Bind to the

### Target Antigen

1. Detection condition setup
Experimental group: B7H3 antigen+phage
Negative control group: BCMA antigen and phage scFv-BCMA
Negative control group 1: other biotin-free antigen (PRPS1)+phage
Negative control group 2: no antigen+phage

### 2. Experimental Protocol

Monoclonal phage and B7H3-02 were separately prepared, and whether they had affinity for the target antigen was preliminarily determined by ELISA experiment chromogenic reaction and the OD value.

### 3. Procedures

Experimental wells and control wells in each group were coated with an equal amount of antigens, and then an equal amount of phages was added. After incubation, unbound phages were removed by multiple times of washing. Phage detection antibodies and secondary antibodies were added, the TMB color development was performed, and the OD₄₅₀ₙₘ reading was measured using a microplate reader.

### 4. Results

The experimental results are shown in Table 2, Table 3, FIG. 2 and FIG. 3, respectively. The results of the chromogenic reaction by ELISA and the OD₄₅₀ₙₘ reading show that clone B7H3-02 could recognize and bind to B7H3 target antigen, B7H3-03 cannot recognize and bind to B7H3 target antigen, and clone B7H3-02 is significantly better than clone B7H3-03.

TABLE 2
Statistical results for B7H3-02

OD_{450 nm}

| | Positive control group BCMA | B7H3-02 |
|---|---|---|
| Experimental group | 0.758 | 0.294 |
| Control group 1 | 0.077 | 0.079 |
| Control group 2 | 0.085 | 0.080 |

TABLE 3
Statistical results for B7H3-03

OD_{450 nm}

| | Positive control group BCMA | B7H3-03 |
|---|---|---|
| Experimental group | 0.885 | 0.095 |
| Control group 1 | 0.081 | 0.081 |
| Control group 2 | 0.083 | 0.088 |

### Example 3. Validation of the Ability of the Fully Human B7H3 scFv (B7H3-02) to Recognize the Target Antigen

### I. Experiment 1

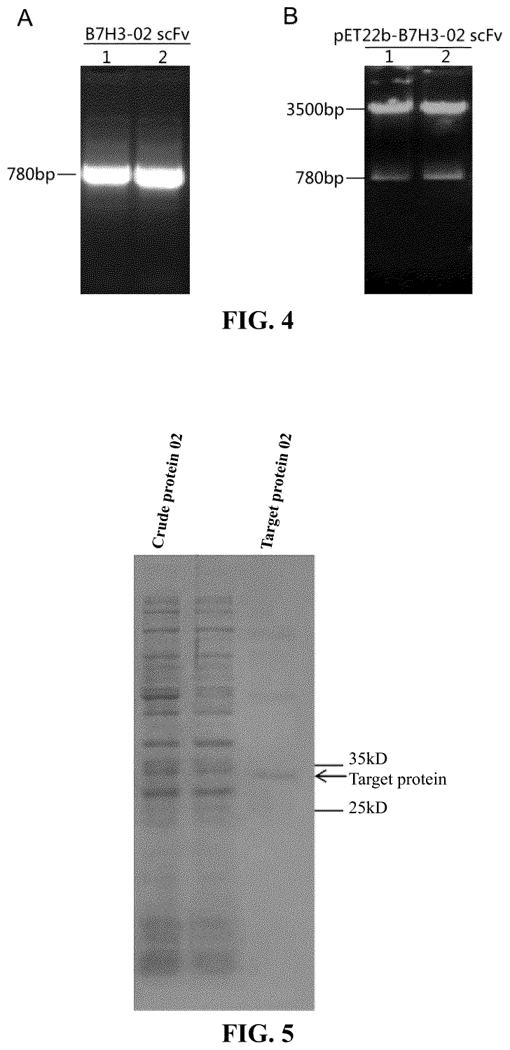

1. scFv Antibody Expression and Purification
pET-22b was used for constructing a B7H3-02 scFv antibody expression vector. The identification results are shown in FIG. 4. Purified B7H3-02 scFv protein was obtained by induced expression and purification. The purification results are shown in FIG. 5.
B7H3-02 scFv antibody: 0.456 μg/μL.
   2. Experimental protocol

Experimental wells and control wells in each group were coated with an equal amount of antigens, and then purified scFv antibodies were added. After incubation, multiple times of washing were performed. His antibodies and secondary antibodies were added, the TMB color development was performed, and the OD₄₅₀ₙₘ reading was measured using a microplate reader.

### 3. Analysis of ELISA Results

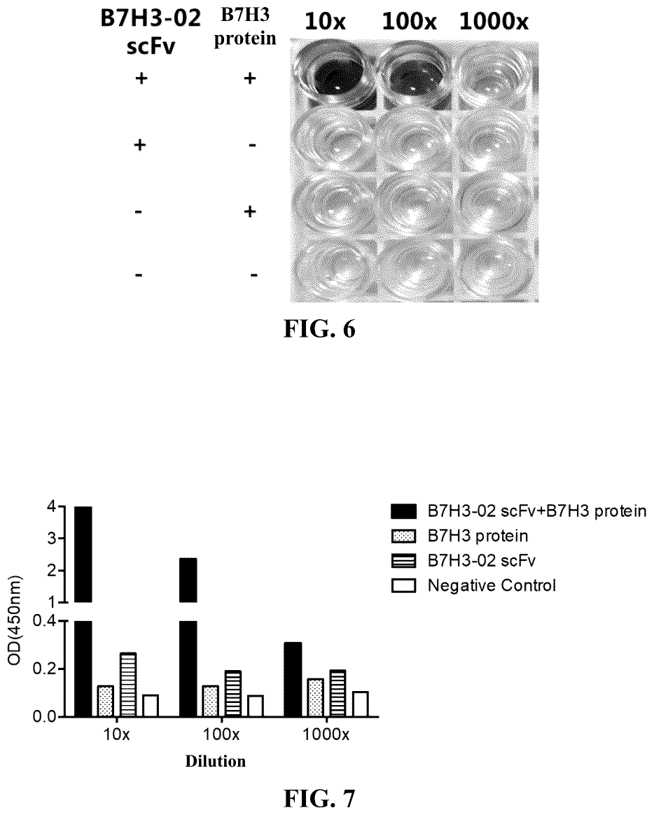

The results are shown in FIG. 6, FIG. 7 and Table 4. B7H3-02 had relatively high affinity, and when the purified scFv antibody was diluted 1000-fold, B7H3-02 still had relatively good affinity.

TABLE 4
Statistical results for affinity

| CD276 (B7H3) | B7H3-02 | | | |
|---|---|---|---|---|
| antigen | antibody | 10x(10 μL) | 100x | 1000x |
| + | + | 3.968 | 2.357 | 0.306 |
| + | − | 0.128 | 0.129 | 0.158 |
| − | + | 0.264 | 0.19 | 0.192 |
| − | − | 0.09 | 0.088 | 0.104 |

### II. Experiment 2

### 1. Experimental Protocol

The preset coupling amount mode was adopted, the purified B7H3-02 antibodies were diluted to 1 μg/mL using a PBS buffer with the pH of 7.5, a biosensor chip Protein A was used to affinity-capture a certain amount of the test antibody, and the unbound activated group was blocked using ethanolamine. The B7-H3 antigen was allowed to flow over the chip surface using a series of concentration gradients. The binding time was 120 s, and the dissociation time was 120 s. After the cycle dissociation was completed, the biochip was washed with glycine-hydrochloric acid (pH=1.5) buffer, the sample injection was performed at 30 μL/min, and regeneration was performed for 30 s. Reaction signals were detected in real time using a Biacore instrument, and thus binding and dissociation curves were obtained.

### 2. Analysis of Results

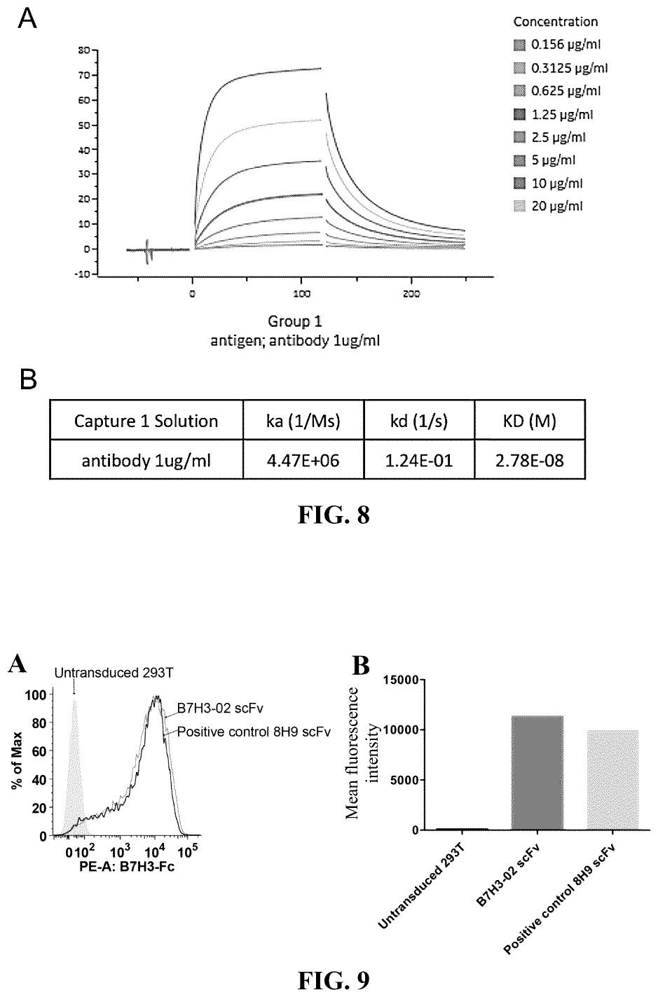

The data obtained from the experiment were fitted with the Langmuir model using GE software. The experimental results are shown in FIG. 8, and the results show that the affinity of the B7H3-02 antibody and the B7H3 antigen was 27.8 nM, indicating that the B7H3-02 antibody and the B7H3 antigen had relatively good affinity.

### III. Experiment 3

### 1. Experimental Protocol

The B7H3-02 scFv was constructed into a eukaryotic expression vector containing a GPI anchor sequence and transfected into 293T cells. Whether the scFv expressed on the surface of the cell membrane could bind to the target antigen was detected by flow cytometry by B7H3-Fc (R&D systems, 1027-B3-100) and PE-Anti-Human IgG Fc (Thermo, 12-4998-82).

### 2. Analysis of Results

The flow cytometry results show that the B7H3-02 scFv could recognize and bind to the B7H3 target antigen, wherein the B7H3-02 scFv had relatively strong binding capacity, which was equivalent to the binding capacity of the positive control 8H9 clone scFv to the antigen (see FIG. 9 panel A). The B7H3-02 scFv was relatively strong by analyzing the mean fluorescence intensity of the cell surface scFv bound to the target antigen B7H3 (see FIG. 9 panel B). The results show that the scFv was bound to a relatively large number of target antigens, namely a relatively large number of fluorescent groups on the surface of each cell membrane. The above results all show that the B7H3-02 scFv had high affinity and strong specificity for the B7H3.

### Example 4. Screening for scFv Targeting CD276 (B7H3-01)

### 1. Experimental Condition Setup

Experimental group: CD276 antigen+CD276-Phage
Control group 1: other biotin-free antigen (PRPS1)+CD276-Phage
Experimental group 2: no antigen+CD276-Phage

### 2. Method

After four rounds of screenings, specific binding antibody sequences were enriched, wherein the total amount of phage input, the quantity of antigens added, and the reaction time, among other conditions, were varied in each round.

The final results were obtained by counting the number of infectious phages contained in the eluate of 0.1 M HCl (PH=2.0) per 100 μL of the control group and the experimental group, and the enrichment was determined.

3. Analysis of screening results

Enrichment occurred in the third round of screening, and the ratio of the number of the phages (specific binding of antigen and antibody) eluted in the experimental group to the control group (non-specific binding between antigen and antibody, or no affinity) was close to 10 times; after the experimental conditions were changed in the fourth round, the experimental group and the control group still kept 10-fold difference, indicating that the screened phage is supposed to have scFv with affinity for the CD276 target protein.

4. scFv Sequence Analysis
24 monoclones were picked for sequencing, 13 of which expressed scFv sequences in their entirety, and different sequences were enriched clone 01: VH:IGHV3-23*04, IGHJ4*02; VK:IGKV1-39*01/IGKVID-39*01, IKJ1*01;
clone 03: VH:IGHV3-33*06, IGHJ6*03; VL:IGKV2-14*01, IGLJ2*01/IGLJ3*01;
the amino acid sequence of scFv (B7H3-01) of clone 01 is set forth in SEQ ID NO: 26, and the nucleotide sequence is set forth in SEQ ID NO: 28 by sequencing;
the amino acid sequence of scFv of clone 03 is set forth in SEQ ID NO: 70 by sequencing.

### Example 5. Detection of the Ability of the scFv Targeting CD276 (B7H3-01) to Bind to the

### Target Antigen

### I. Experiment 1

### 1. Detection Condition Setup

Experimental group: antigen+phage
Positive control group: BCMA antigen and phage scFv-BCMA
Negative control group 1: other biotin-free antigen (PRPS1)+phage
Negative control group 2: no antigen+phage

### 2. Experimental Protocol

Preparation of monoclonal phage: CD276-01 and CD276-03, and whether they had affinity for the target antigen was preliminarily determined by ELISA experiment chromogenic reaction and the OD value.

### 3. Procedures

Experimental wells and control wells in each group were coated with an equal amount of antigens, and then an equal amount of phages was added. After incubation, unbound phages were removed by multiple times of washing. Phage detection antibodies and secondary antibodies were added, the TMB color development was performed, and the OD₄₅₀ₙₘ reading was measured using a microplate reader.

### 4. Analysis of Results

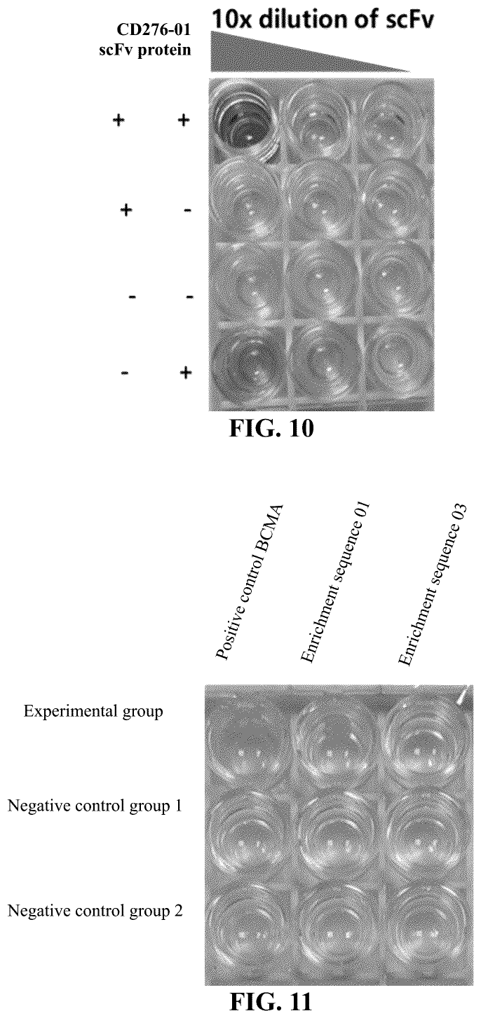

The results are shown in FIG. 10 and Table 5. The chromogenic reaction by ELISA and the OD 450 nm reading show that clone CD276-01 could recognize and bind to CD276 target antigen, and CD276-03 could not recognize the target antigen.

TABLE 5
Statistical results for CD276-01 and CD276-03

OD_{450 nm}

| | Positive control | | |
|---|---|---|---|
| | group BCMA | CD276-01 | CD276-03 |
| Experimental group | 0.885 | 0.569 | 0.095 |
| Control group 1 | 0.081 | 0.096 | 0.081 |
| Control group 2 | 0.083 | 0.086 | 0.088 |

### II. Experiment 2

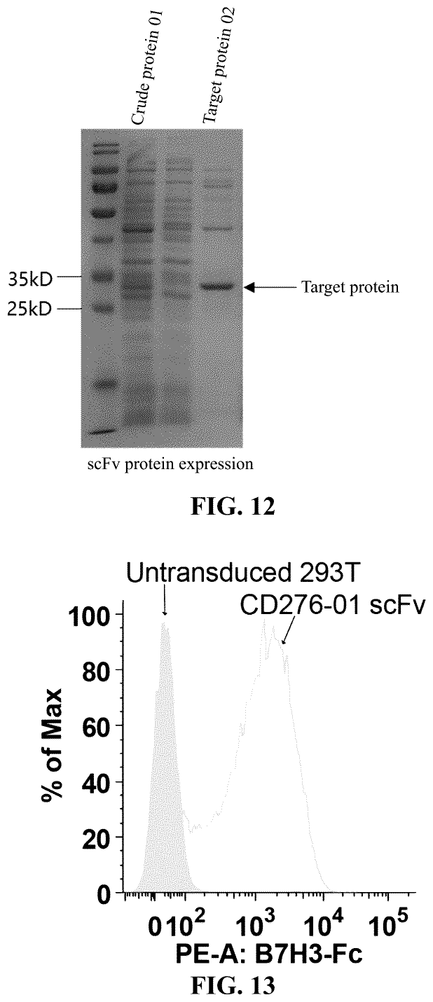

1. scFv Antibody Expression and Purification pET-22b was used for constructing a CD276-scFv antibody expression vector. Two purified scFv proteins were obtained by induced expression and purification. The purification results are shown in FIG. 12.
CD276-01 scFv antibody: 0.474 μg/μL.

### 2. Experimental Protocol

Experimental wells and control wells in each group were coated with an equal amount of antigens, and then purified scFv antibodies were added. After incubation, multiple times of washing were performed. His antibodies and secondary antibodies were added, the TMB color development was performed, and the OD₄₅₀ₙₘ reading was measured using a microplate reader.

### 3. Analysis of ELISA Results

The results are shown in FIG. 11 and Table 6. When the purified scFv antibody was diluted 1000-fold, CD276-01 still had a certain target antigen binding ability.

TABLE 6
Statistical results for affinity

| CD276 antigen | scFv-01 antibody | 10x(10 μL) | 100x | 1000x |
|---|---|---|---|---|
| + | + | 2.063 | 0.353 | 0.156 |
| + | − | 0.109 | 0.105 | 0.11 |
| − | + | 0.767 | 0.378 | 0.112 |
| − | − | 0.111 | 0.107 | 0.113 |

### III. Experiment 3

### 1. Experimental Protocol

The CD276-01 scFv was constructed into a eukaryotic expression vector containing a GPI anchor sequence and transfected into 293T cells. Whether the scFv expressed on the surface of the cell membrane could bind to the target antigen was detected by flow cytometry by CD276-Fc (R&D systems, 1027-B3-100) and PE-Anti-Human IgG Fc (Thermo, 12-4998-82).

### 2. Analysis of Results

The CD276-01 scFv could recognize and bind to the CD276 target antigen by flow cytometry results (FIG. 13).

### Example 6. Preparation of B7H3-CAR-T (B7H3-02) with High Expression of hSki

### 1. Method

(1) isolation of PBMC cell
1) peripheral blood of healthy volunteers was collected and centrifuged at 1300 g at room temperature for 10 min. Then the plasma part was discarded, and the remaining blood cells were diluted and mixed well using an equal volume of normal saline;
2) the blood cell suspension was slowly added into the upper layer of a lymphocyte isolation solution, and centrifuged at 600 g at room temperature for 25 min;
3) the lymphocytes in the middle buffy coat layer were pipetted and added with normal saline for washing, red blood cell lysis treatment was performed if necessary, the mixture was centrifuged at 400 g at room temperature for 10 min, and the supernatant was discarded to give PBMC cells.
(2) construction of CAR expression vector
1) an scFv encoding sequence targeting human B7H3 was synthesized, and the scFv comprises a heavy chain VH and a light chain VL linked by a 3×G4S short peptide;
2) a retroviral vector MSCV and the scFv targeting human B7H3 synthesized in step 1) were digested with Nco I and Mlu I, the fragments were recovered, and the recovered target fragments were connected by T4 ligase and then transformed into Stb13 competent cells;
3) a single clone was selected for plasmid extraction, and after enzyme digestion and identification, it was sent for sequencing confirmation. The correct plasmid was MSCV-M13B702.

In the construction method described above, the nucleotide sequence of the heavy chain VH is set forth in SEQ ID NO: 9, the nucleotide sequence of the light chain VL is set forth in SEQ ID NO: 17, the nucleotide sequence of the G4S short peptide is set forth in SEQ ID NO: 23, the amino acid sequence of the constructed CAR expression vector (comprising a signal peptide, T2A and hSki) is set forth in SEQ ID NO: 56, and the nucleotide sequence is set forth in SEQ ID NO: 57.
(3) retrovirus packaging
1) 293T cells were prepared and plated at 3×10⁶/100 mm culture dish;
2) on the next day, 293T cell state was observed. The cells were in a good state, and transfection was performed;
3) the transfection reagent was prepared using a 1.5 mL EP tube: 30 μL Genejuice+470 μL IMDM, and incubated at room temperature for 5 min;
4) the shuttle plasmid MSCV-M13B702 and the helper plasmid pCL-Ampho were added to a new 1.5 mL EP tube according to the total amount of 10 μg and the ratio of 3:2, which was DNA Mix;
5) one part of the transfection reagent was added to the DNA Mix, gently mixed well, and incubated at room temperature for 15 min;
6) culture dishes were marked, the reagent obtained in the previous step was added to the culture dishes respectively, and the virus supernatant was collected after 48-72 h;
7) the supernatant was subpackaged in 1.5 mL EP tubes, each tube being 1 mL, and stored in a refrigerator at −80° C. for later use.

### (4) Retrovirus Transduction

1) Day −1: a 24-well plate was coated with hCD3/CD28 antibodies;
2) Day 0: human PBMCs were thawed, counted, and resuspended in L500 medium (L500+10% FBS+1% P.S., and cytokines 5 ng/ml IL-15 and 10 ng/ml IL-7 were added during CAR-T cell preparation) to 1×10⁶/mL. The coating solution was discarded. Each well was inoculated with 1 mL of cells;
3) Day 1: the 24-well plate was coated with 1 μg/mL Retronectin;
4) Day 2: after 48 h of cell activation, CAR virus infection was performed. The cells were collected into a centrifuge tube, counted, and distributed according to (0.5-1)×10⁶ cells per tube. The tube was centrifuged, and the supernatant was discarded. T cells were resuspended with 1 mL of virus solution, and the T cells were inoculated onto the 24-well plate and centrifuged at 1500 g at 30° C. for 2 h. The supernatant was gently discarded, and L500 medium comprising cytokines was slowly added.
   (5) Amplification of B7H3-CAR-T Cell with High Expression of hSki

Day 4-Day 14: the medium was supplemented to maintain the cell density at (0.5-1)×10⁶/mL depending on the cell growth and cell number.

### (6) Detection of CAR Expression Efficiency

Day 4: the T cell purity and the CAR positive rate were detected by flow cytometry. The cells were labeled by B7H3-Fc protein, incubated at room temperature for 20 min, and washed. Then the PE-Anti Human IgG-Fc antibody was added, incubated at room temperature for 20 min in the dark, and washed. Finally, APC-CD3 staining was performed, and analysis was performed using a flow cytometer.

### 2. Results

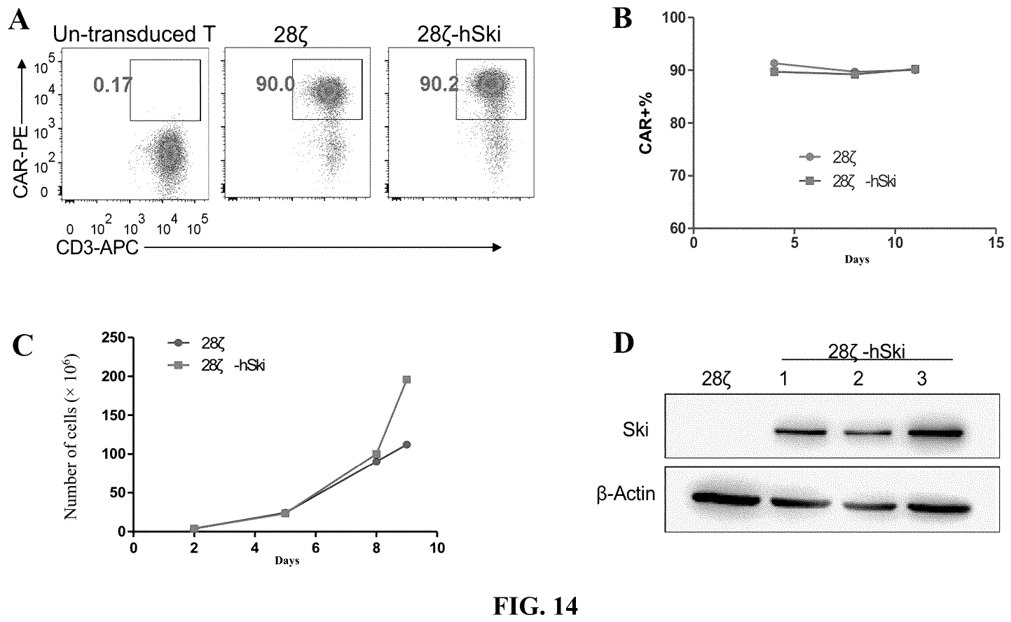

The results of the experiment are shown in FIG. 14, and the results show that: the B7H3-CAR-T cell constructed by the present application comprises the Ski gene, indicating that the present application successfully constructed a fully human CAR comprising the human Ski gene targeting B7H3, and further prepared the B7H3-CAR-T cell. The results of the flow cytometer analysis show that: the positive rate of CAR expression in the B7H3-CAR-T cells with high expression of hSki was up to 90%. Ski was efficiently expressed in the prepared CAR-T cells, and the expression of Ski not only did not influence the positive rate of CAR expression, but also promoted the proliferation of B7H3-CAR-T cells.

### Example 7. B7H3-CAR-T Cell with High Expression of hSki Effectively Antagonizing TGF-β Immunosuppression

### 1. Method

(1) 24-well plate. The number of the required well plates was determined according to experiment needs. The tumor cells were digested, resuspended, and seeded at 50000 cells per well, and at this time, an L500 basal culture medium added with serum and a diabody was applied;
(2) after the tumor cells adhered to the wall (about 5 h), different groups of CAR-T were sequentially added in effector-to-target ratios of 1:1, 1:2.5, and 1:5 (CAR-T positive rate was detected in advance and was ensured to be prepared in the same batch), and an experimental control group added with 3 ng/mL TGF-β was set, the volume of each well being made up to 2 mL (using an L500 basal culture medium added with serum and a diabody);
(3) the mixed suspension of each tube and the T cells with a corresponding effector-to-target ratio was collected, labeled and stained using an APC-CD3 antibody. The initial ratio of the tumor cells to CAR-T under the condition of different effector-to-target ratios in different groups was detected by flow cytometry;
(4) in the process, a microscope was used for observing the killing effect of the CAR-T cells on the tumor cells, and if necessary, a culture medium was supplemented or replaced;
(5) when the tumor was killed to a certain degree and the killing effect was remarkable in the effector-to-target ratio of 1:1, the suspended CAR-T cells of each well were respectively collected and the tumor cells were digested and collected. They were labeled and stained using an APC-CD3 antibody. The ratio of the tumor cells to CAR-T under the condition of different effector-to-target ratios in different groups was detected by flow cytometry;
(6) the flow cytometry results were analyzed using FlowJ.

### 2. Results

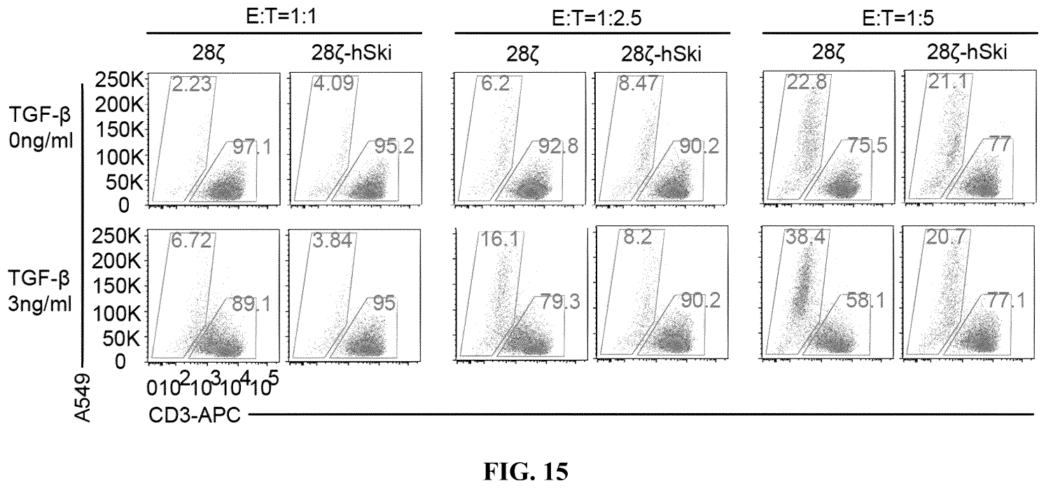

The results are shown in FIG. 15. 28ζ and 28ζ-hSki CAR-T cells were co-incubated with A549 lung cancer cells at different effector-to-target ratios (E:T=1:1, 1:2.5, and 1:5), and treated with or without 3 ng/ml TGF-β. The mixed cell suspensions of the initial three effector-to-target ratios were collected at 0 h, stained with CD3-APC flow antibodies, and the initial ratio of the two cell components was detected by flow cytometer. After 60 h of co-incubation, CAR-T and A549 cells were collected from each experimental sample and stained using CD3-APC antibodies. The CAR-T and A549 cell ratios were analyzed by flow analysis to assess the killing ability of 28ζ and 28ζ-hSki CAR-T cells against A549 tumor cells. The results show that: there was no significant difference in the killing of A549 by 28 and 28ζ-hSki CAR-T cells in the absence of TGF-β; but in the presence of TGF-β, the killing function of the 28ζ CAR-T was significantly inhibited, while the 28ζ-hSki CAR-T was basically not influenced, which shows that the 28ζ-hSki CAR-T cells could antagonize the immunosuppressive action of TGF-β, efficiently kill tumor cells, and have better killing effect in a solid tumor microenvironment.

### Example 8. Secretion of IFN-γ by B7H3-CAR-T Cell with High Expression of hSki

### 1. Method

(1) 12-well plate. The number of the required well plates was determined according to experiment needs. The tumor cells were digested, resuspended, and seeded at 150000 cells per well, and at this time, an L500 basal culture medium added with serum and a diabody was applied;
(2) after the tumor cells adhered to the wall (about 5 h), different groups of CAR-T were sequentially added in effector-to-target ratios of 1:1, 1:2.5, and 1:5 (CAR-T positive rate was detected in advance and was ensured to be prepared in the same batch), and an experimental control group added with 3 ng/mL TGF-β was set, the volume of each well being made up to 2 mL (using an L500 basal culture medium added with serum and a diabody);
(3) a blank control well was set, and 28ζ and 28-hSki CAR-T cells were separately cultured according to the number of CAR-T cells when the effector-to-target ratio was 1:1 under the same culture conditions of step (2) to serve as difference control before and after co-culture;
(4) after 48 h, 200-500 μL of the supernatant from co-culturing CAR-T cells and tumor cells from each group was collected into an EP tube (the cells should be avoided being collected as much as possible, and the supernatant was collected by centrifugation if necessary), the name and the time were marked, and the tube was cryopreserved in a refrigerator at −80° C. for later use;
(5) an experimental plate was coated with Capture mAb 1-Dlk one day in advance, which was diluted to a final concentration of 2 μg/mL with PBS with the pH of 7.4 at 100 μL per well, and left overnight at 4° C.;
(6) the pre-coated plate was washed twice with PBS (200 μL/well);
(7) PBS containing 0.05% Tween-20 and 1% BSA was added at 200 μL/well, and the plate was incubated at room temperature for 1 h;
(8) the aliquoted IFN-γ standard (1 μg/mL) that was taken out in advance and thawed on ice and a sample to be detected were treated; the IFN-γ standard was diluted to seven gradient concentrations, namely 500, 250, 125, 62.5, 31.2, 15.6, and 7.8 pg/mL, and the sample to be detected was 50-fold diluted; both the standard and the sample were diluted using PBS containing 0.05% Tween-20 and 1% BSA;
(9) the plate was washed with PBS containing 0.05% Tween-20 for 5 times and left to stand for 1 min after each addition. The washing liquid was knocked off in a waste liquid tank, and then fully discarded on absorbent paper. Cross-contamination between wells was avoided during the procedure;
(10) the diluted standard and the sample to be detected were added into the well in sequence at 100 μL/well, and incubated at room temperature for 2 h;
(11) the operation of step (9) was repeated;
(12) 100 μL of 1 μg/mL Detection mAb 7-B6-1 was added and incubated at room temperature for 1 h (diluted using PBS containing 0.05% Tween-20 and 1% BSA);
(13) the operation of step (9) was repeated;
(14) 100 μL of 1:1000 diluted Streptavidin-HRP was added and incubated at room temperature for 1 h (diluted using PBS containing 0.05% Tween-20 and 1% BSA);
(15) the operation of step (9) was repeated;
(16) 100 μL of a TMB substrate solution was added, and the chromogenic reaction was observed;
(17) the observation was for about 10 min. A stop solution was added to stop the reaction when the high-response well showed dark blue, and the blue was changed into yellow; preparation of the stop solution: 9.1 mL ddH₂O+1 mL concentrated sulfuric acid;
(18) the optical density (OD value) of each well under the wavelength of 450 nm was detected using a microplate reader;
(19) the data were copied and analyzed.

### 2. Results

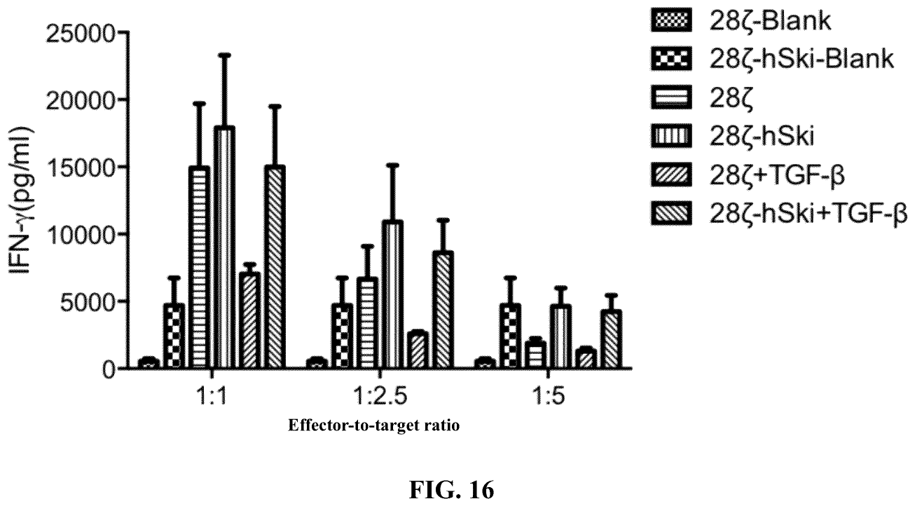

The results are shown in FIG. 16. 28ζ and 28ζ-hSki CAR-T cells were co-incubated with A549 lung cancer cells at different effector-to-target ratios (E:T=1:1, 1:2.5, and 1:5), and treated with or without 3 ng/ml TGF-β. The mixed cell culture supernatant was directly collected at 0 h as a blank group (Blank). After 48 h, the culture supernatant was collected again, and the content of IFN-γ in the supernatant was measured by ELISA. The results show that: before contacting the target cells, the IFN-γ secretion capacity of the 28ζ-hSki CAR-T cells was significantly higher than that of the 28ζCAR-T cells; after co-incubation with the target cell A549, the IFN-γ secretion by 28ζ and 28ζ-hSki CAR-T cells was significantly increased, indicating that the CAR-T cells were activated; but when TGF-β was present, IFN secretion by 28ζ CAR-T cells was significantly reduced, while 28ζ-hSki CAR-T cells still maintained higher levels of IFN-γ secretion. It is further indicated that 28g-hSki CAR-T cells can antagonize the immunosuppressive effects of TGF-β.

### Example 9. Validation of In Vivo Clearance of Lung Cancer Subcutaneously Transplanted Tumor by the B7H3-CAR-T Cell with High Expression of hSki

### 1. Method

(1) 4-6 weeks old NCG female mice were injected subcutaneously 150 μL of cell suspension containing 1×10⁷ human lung cancer cell A549 into the right dorsal side of the mice;
(2) the growth condition of the subcutaneously transplanted tumor was continuously observed, and when the tumor body gradually enlarged, the long diameter (a) and the short diameter (b) of the tumor body were measured using a vernier caliper. The volume of the tumor body is a×b²/2;
(3) when the tumor body size was about 100-200 mm³, the mice were randomly divided into 5 groups;
(4) the prepared 28ζ and 28ζ-hSki CAR-T cells were respectively administered to tumor-bearing mice for tail vein injection treatment according to the dose of 2×10⁶/100 μL and 5×10^{6/100} μL, and PBS group was used as a control;
(5) every 3 to 4 days, the body weight of the mice and the change of the volume of the beared tumor were measured, and the comprehensive condition in the treatment process was observed.

### 2. Results

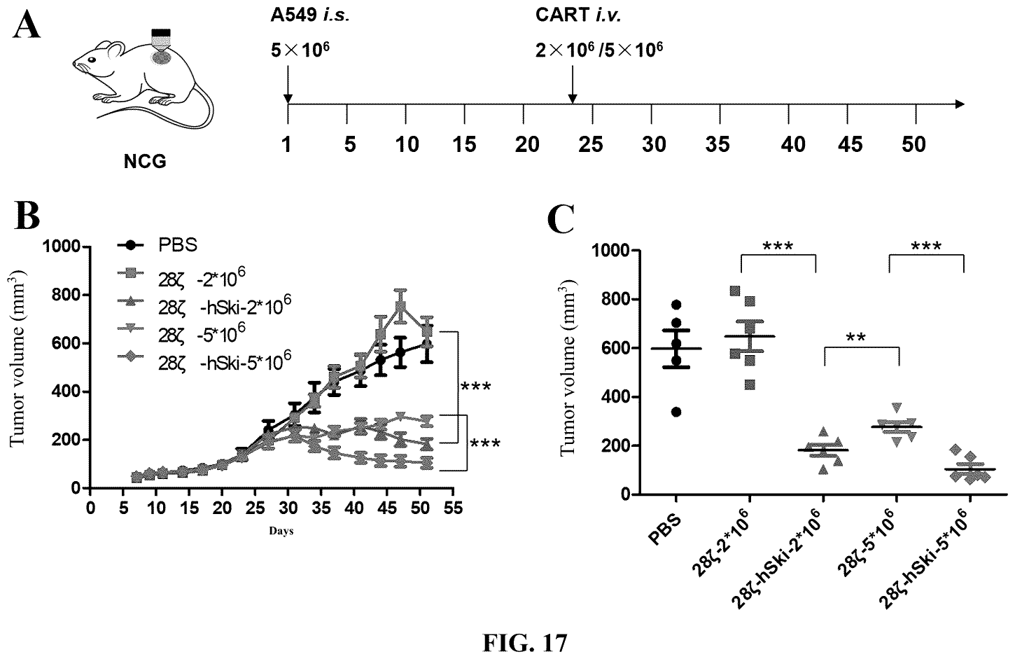

The experimental results are shown in FIG. 17. A lung cancer NCG mouse subcutaneously transplanted tumor model was established. When the volume of the beared tumor of the mice was 100-200 mm³, the mice were randomly divided into 5 groups (PBS, 2×10⁶ 28ζ, 5×10⁶ 28ζ, 2×10⁶ 28ζ-hSki, 5×10⁶ 28ζ-hSki) of 6, and administered with a tail vein injection of 2×10⁶ or 5×10⁶ of a therapeutic dose of 28ζ or 28ζ-hSki CAR-T cells. The PBS group was the control group. The body weight of the mice, the change of the volume of the beared tumor, and the comprehensive condition in the treatment process were continuously detected. From the formation of the subcutaneously transplanted tumor, the body weight of the mice and the size change of the transplanted tumor were measured and recorded every three days. The volume of the transplanted tumor was calculated, and the tumor growth curve was drawn according to the time axis. The results show that: the 28g-hSki CAR-T cells could kill lung cancer transplanted tumor with high efficiency under the condition of a relatively low dose, indicating that the tumor killing effect of the 282-hSki CAR-T cells was significantly better than that of the 282 CAR-T cells.

### Example 10. Preparation of the Fully Human B7H3-CAR-iNKT (comprising IL-15) (B7H3-02)

### 1. Method

(1) Preparation of iNKT
1) isolation of PBMCs: peripheral blood of a donor was collected, and the whole blood was diluted with an equal amount of normal saline. A lymphocyte isolation solution and the diluted blood were added into a centrifuge tube according to the ratio of 1:2 and centrifuged at 2000 rpm/min for 20 min. The cells in the buffy coat layer were collected, washed twice with normal saline, and centrifuged at 1500 rpm/min for 8 min to give peripheral blood mononuclear cells PBMCs;
2) induction of iNKT cells: the PBMCs were resuspended with lymphocyte culture medium and adjusted to a concentration of 2×10⁶/mL. α-Galcer, IL-2, IL-21, IL-4, and GM-CSF were added. The cells were inoculated onto a 24-well plate and placed in an incubator at 37° C. with 5% CO₂. The cell state was observed every day, and half of the medium was exchanged every other day;
3) magnetic bead sorting iNKT cells: the induced cells were collected on day 10 and resuspended using 500 μL of MACS buffer. Anti-iNKT MicroBeads were added according to the amount in the instruction, mixed well, placed at 4° C., and incubated for 30 min. 5 mL of MACS buffer was added for washing, the mixture was centrifuged at 400×g for 5 min, and the supernatant was discarded; the cells were resuspended using 500 μL of MACS buffer, loaded onto a LS sorting column, washed 3 times by MACS buffer, 3 mL each time; finally, the sorting column was placed in a collecting tube, and 500 μL of MACS buffer was added for elution to give iNKT positive cells;
4) activation and amplification of iNKT cells: the resulting cells in the previous purification step were resuspended using lymphocyte culture medium containing IL-7 and IL-15 on day 10, inoculated on CD3Ab and CD28Ab pre-coated plates, and placed in an incubator at 37° C. with 5% CO₂ for extensive amplification.

### (2) Construction of CAR Expression Vector

1) an scFv encoding sequence targeting human B7H3 was synthesized, and the scFv comprises a heavy chain VH and a light chain VL linked by a G4S short peptide;
2) a retroviral vector MSCV and the synthesized scFv targeting human B7H3 were digested with Nco I and Mlu I, the fragments were recovered, and the recovered target fragments were connected by T4 ligase and then transformed into Stb13 competent cells;
3) a single clone was selected for plasmid extraction, and after enzyme digestion and identification, it was sent for sequencing confirmation. The correct plasmid was B7H3.CAR;

In the construction method described above, the amino acid sequence of the chimeric antigen receptor targeting B7H3 (comprising signal peptide, T2A, and IL-15) is set forth in SEQ ID NO: 58, and the nucleotide sequence is set forth in SEQ ID NO: 59.

### (3) Retrovirus Packaging

6 μg of a shuttle plasmid MSCV-B7H3.CAR comprising a CAR structure and 4 μg of a helper plasmid pCL-Ampho were mixed in 300 μL of opti-MEM culture medium. 30 μL of Genejuice transfection reagent was added dropwise in another 300 μL of opti-MEM culture medium, gently mixed well, and left to stand at room temperature for 5 min. The mixture containing the transfection reagent was added dropwise into the plasmid mixture, mixed well by shaking, and left to stand at room temperature for 15 min. Then PEI and the plasmid mixture were added dropwise into pre-plated 293T cell culture dish, and mixed well by gentle shaking. After 48-72 h, the supernatant was collected, filtered through a 0.45 μm syringe filter, and stored in an ultra-low temperature refrigerator for later use.

(4) Viral Infection of iNKT Cells

B7H3.CAR virus liquid was added into 10 μM HEPES and 6-8 μg/mL polybrene, and mixed well. Activated iNKT cells were resuspended using the virus liquid, then added into a 24-well plate pre-coated with RetroNectin, and centrifuged at 1500 g at 30° C. for 2 h. Then the supernatant was removed. X-Vivo culture medium containing 5% fetal bovine serum, 200 U/mL IL-2, 10 ng/ml IL-7, and 5 ng/ml IL-15 was supplemented. The amplification and culture was continued to give B7H3.CAR-iNKT cells.

### (5) Detection of CAR Expression Efficiency

After 48-72 h of virus infection, 2×10⁵ cells were taken for staining. 1 μg/mL of B7H3-Fc protein (R&D, 1027-B3-100) was firstly added and incubated at 4° C. for 30 min. The cells were washed and then the AF647-anti-human IgG antibody (Jackson, 109-606-088) was added and incubated in the dark at 4° C. for 30 min. The cells were washed and finally PerCP/Cy5.5-CD3 (Biolegend, 317336), PE-INKT (BD, #552825), and an antibody were added and incubated in the dark at 4° C. for 30 min. After washing, the mixture was loaded on the machine for assay.

### 2. Results

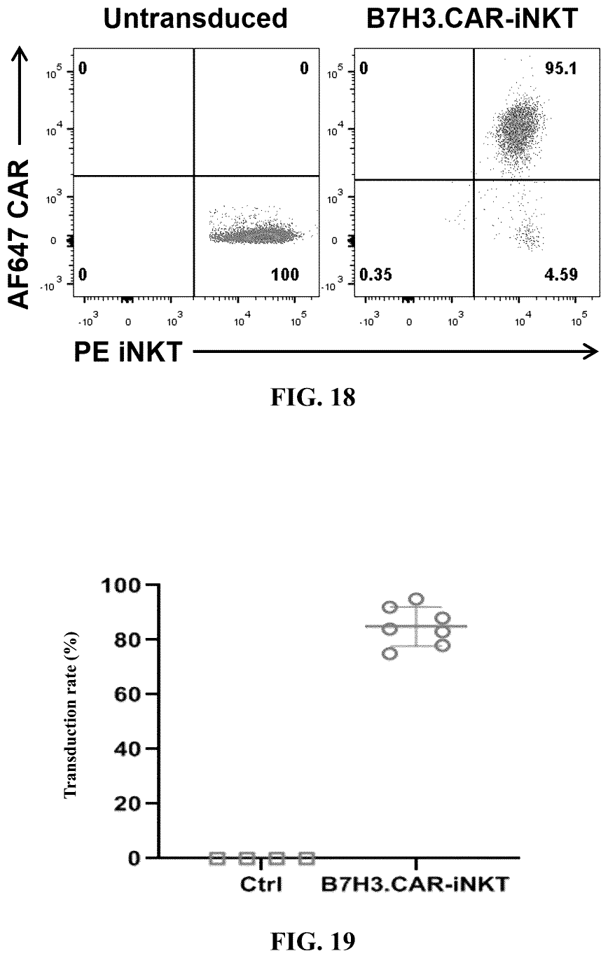

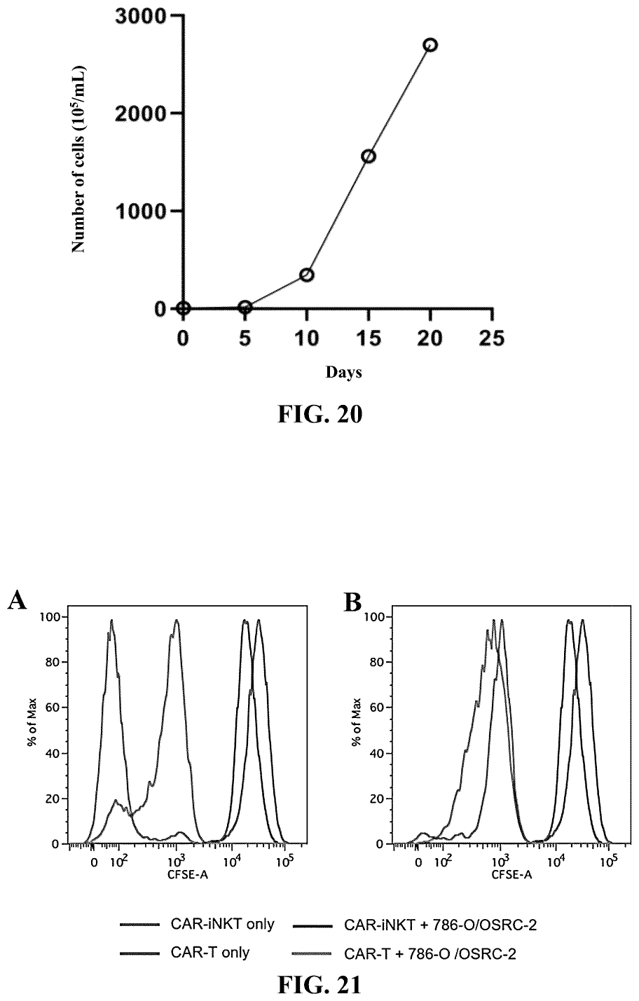

CAR transfection efficiency of the B7H3.CAR-iNKT (comprising IL-15) cells prepared above was assayed by flow cytometry, and the results show that the CAR transfection efficiency was up to 75-95%. FIG. 18 is a flow cytometric representative plot and FIG. 19 is a statistical graph; the results of FIG. 20 show that the B7H3-CAR-iNKT cells prepared by the present application could effectively express CAR targeting B7H3, could be extensively amplified to ˜108, and could meet the clinical requirement.

### Example 11. Validation of the Ability of the Fully Human B7H3-CAR-iNKT Cell to Kill Kidney Cancer Cell In Vitro

### 1. Method

### (1) CFSE Experiment for Detecting Proliferation Capacity

CFSE staining: B7H3.CAR-iNKT cells were collected, washed with 0.1% FBS/PBS, and resuspended. A CFSE working solution was added for staining to a final concentration of 1.5 μM, and incubated at room temperature for 10 min. FBS was added and incubated at 37° C. for 10 min, and the staining was stopped. Then the cells were washed twice with 2% FBS/PBS, and finally suspended with a T cell culture medium for later use;

Kidney cancer cells 786-O and OSRC-2 were plated overnight. The stained effector cells described above were added according to an effector-to-target ratio of 1:2, and an individual effector cell group was used as a control. After 5 days, the cells were collected, washed, and detected for CFSE fluorescence signals by flow cytometry. Proliferation capacity of the B7H3.CAR-iNKT cells were analyzed.

### (2) Detection of Cytokine Release Ability Using ELISA Kit

1×10⁵ β7H3.CAR-iNKT cells were collected, respectively mixed well with 1×10⁵ kidney cancer cells 786-O and OSRC-2, and added to a 24-well plate for co-incubation. A replicate well was set. After 24 h, the culture supernatant was collected; the content of IFN-γ and IL-2 was detected using an ELISA kit.

### (3) Detection of Killing Effect by RTCA Real-Time Label-Free Dynamic Cell Analysis Technology

Firstly, 50 μL DMEM complete culture medium was added to an E-Plate detection plate of an xCELLigence cell function analyzer, and the background impedance value was measured; target cells in logarithmic phase were collected, the concentration of the cell suspension was adjusted to 1×10⁵/mL, 100 μL of the cell suspension was added to the E-Plate detection plate, and the plate was left to stand at room temperature for 30 min and placed on a detection platform; real-time dynamical observing was performed, when the proliferation of the target cells was in a plateau, 50 μL of effector cells were added to the experimental well according to effector-to-target ratios of 1:1 and 1:5, and only T cell culture medium was added in the control group; B7H3.CAR-iNKT and cell-mediated cell killing effect curve were observed in real time.

### 2. Results

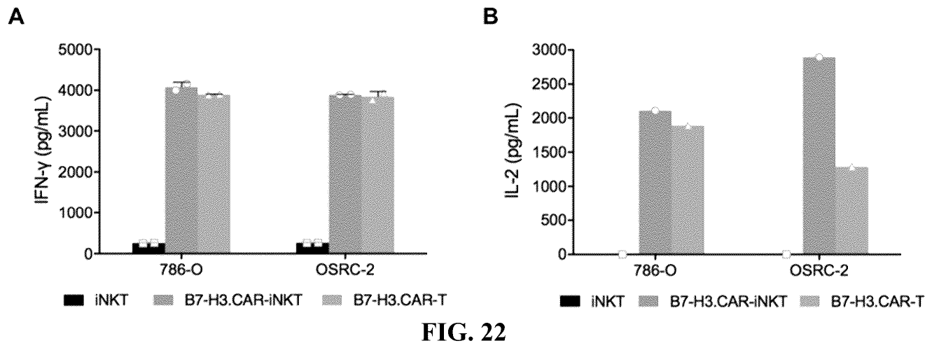

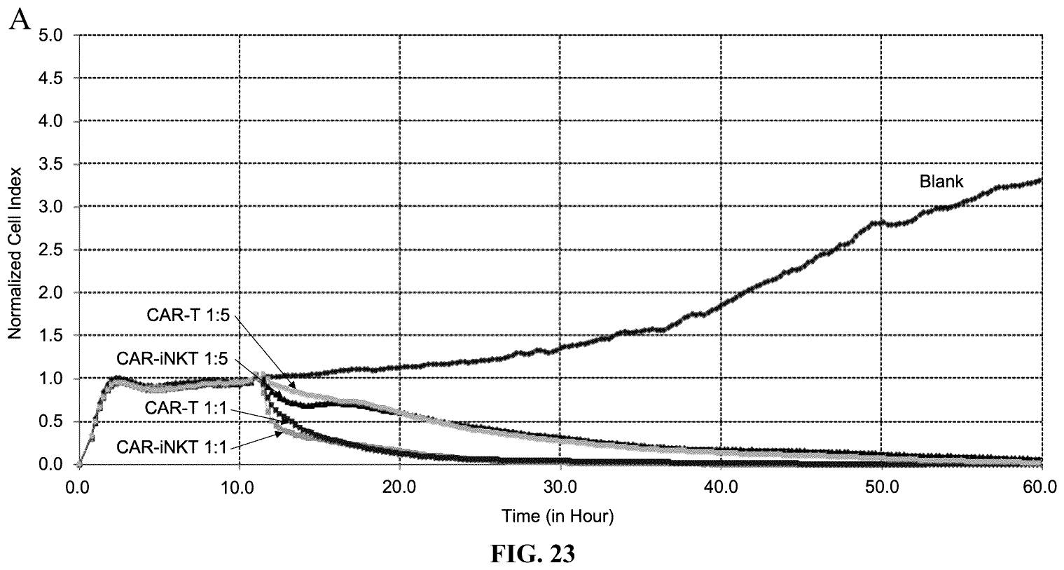

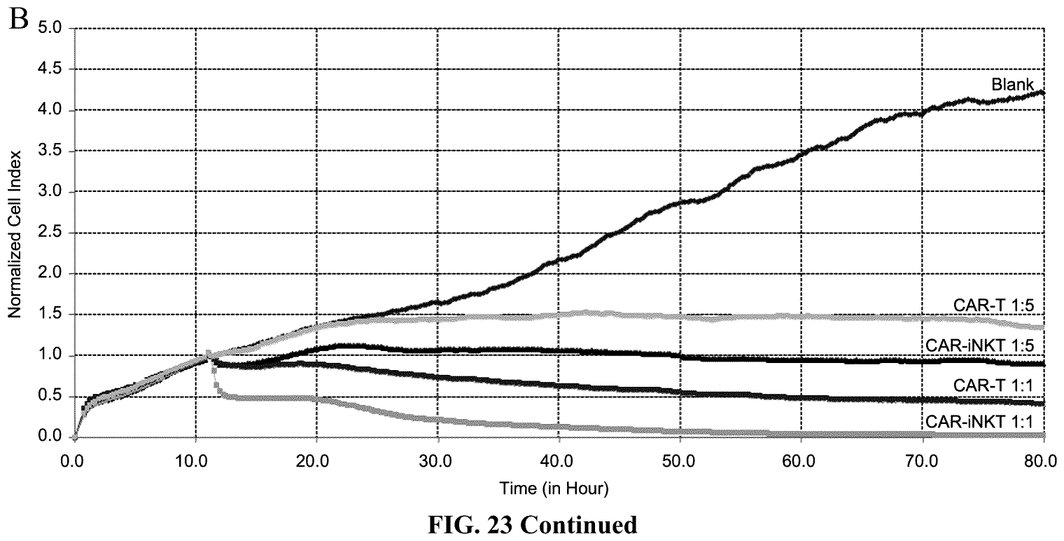

The results of the experiment are shown in FIGS. 21-23, and the results of FIG. 21 show that: the B7H3.CAR-iNKT (comprising IL-15) cells prepared by the present application had relatively strong proliferation capacity; the results of FIG. 22 show that: compared with the B7H3.CAR-T, the B7H3.CAR-iNKT cells prepared by the present application could secrete higher-level cytokines; the results of FIG. 23 show that: the killing effect of the B7H3.CAR-iNKT and the B7H3.CAR-T in the same effector-to-target ratio had no significant difference.

### Example 12. Validation of In Vivo Clearance of Kidney Cancer Transplanted Tumor by the Fully Human B7H3-CAR-iNKT Cell

### 1. Method

6 weeks old male NCG mice were purchased. A mouse kidney cancer subcutaneously transplanted tumor model was constructed by a subcutaneous injection of 2×10⁶ OSRC-2-Ffluc-GFP. On day 12, after the tumor was formed, the mice were randomly grouped into Ctrl group, iNKT group, and B7H3.CAR-iNKT group, with 5 mice in each group, and 3 groups in total; on days 0 and 8, treatment was performed by tail vein infusion of iNKT and B7H3.CAR-iNKT cells, respectively, 5×10⁶/mice; only PBS was infused in Ctrl group; treatment effect was observed twice a week by measuring tumor volume, survival of CAR-iNKT in vivo was detected by blood sampling via submaxillary vein, and survival of the mice was recorded.

### 2. Results

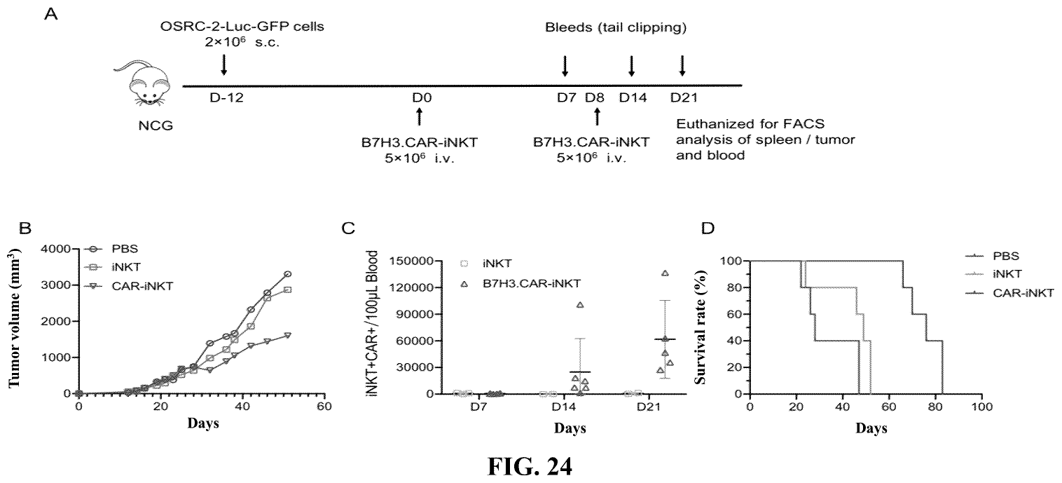

The results of the experiment are shown in FIG. 24, and the results of FIG. 24 panel A show: establishment of NCG mouse kidney cancer subcutaneously transplanted tumor model and treatment pattern diagram using B7H3.CAR-iNKT cells; the results of FIG. 24 panel B show that: compared with PBS and iNKT cell groups, the B7H3.CAR-iNKT cells were capable of inhibiting kidney cancer; the results of FIG. 24 panel C show that: 14 and 21 days after treatment, the CAR-iNKT cells in peripheral blood of the mice in the B7H3.CAR-iNKT treatment group were higher than those of the control group; the results of FIG. 24 panel D show that the survival of the mice in the B7H3.CAR-iNKT treatment group was significantly prolonged.

### Example 13. Validation of the Ability of the Fully Human B7H3-CAR-iNKT Cell to Kill Ovarian Cancer Cell In Vitro

### 1. Method

Firstly, 50 μL DMEM complete culture medium was added to an E-Plate detection plate of an xCELLigence cell function analyzer, and the background impedance value was measured; ovarian cancer cells SKOV-3 in logarithmic phase were collected, the concentration of the cell suspension was adjusted to 1×10⁵/mL, 100 μL of the cell suspension was added to the E-Plate detection plate, and the plate was left to stand at room temperature for 30 min and placed on a detection platform; real-time dynamical observing was performed, when the proliferation of the target cells was in a plateau, 50 μL of effector cells were added to the experimental well according to effector-to-target ratios of 5:1, 1:1, and 1:5, and only T cell culture medium was added in the control group; B7H3.CAR-iNKT and cell-mediated cell killing effect curve were observed in real time.

### 2. Results

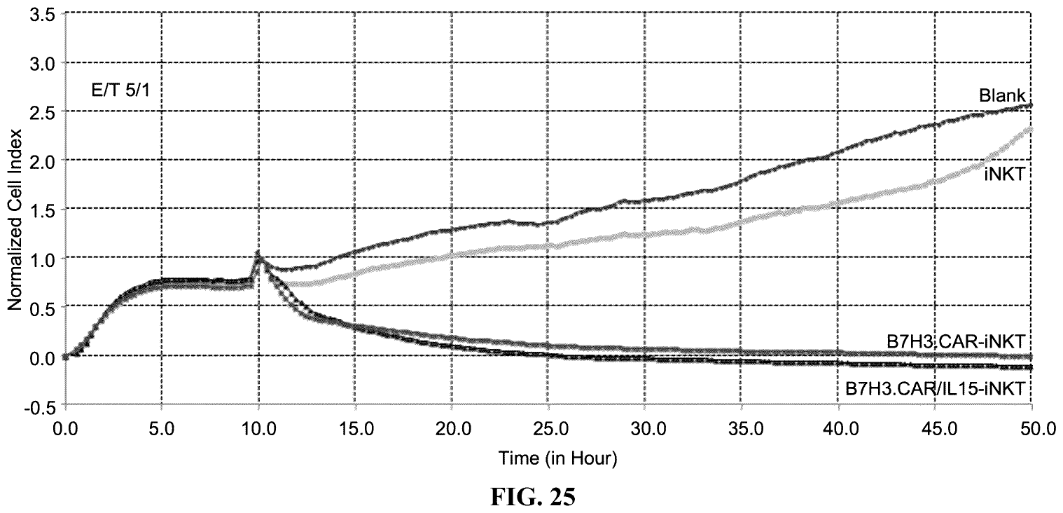

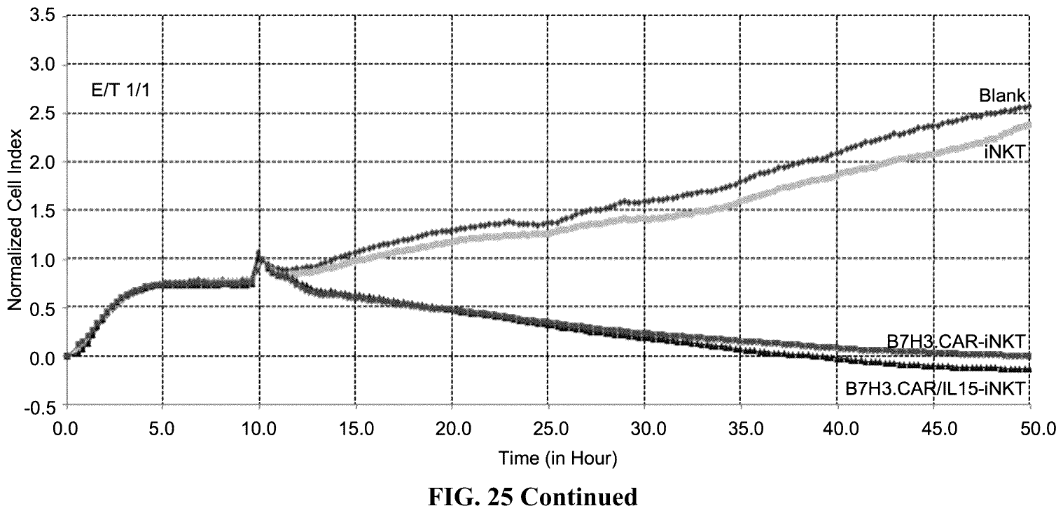

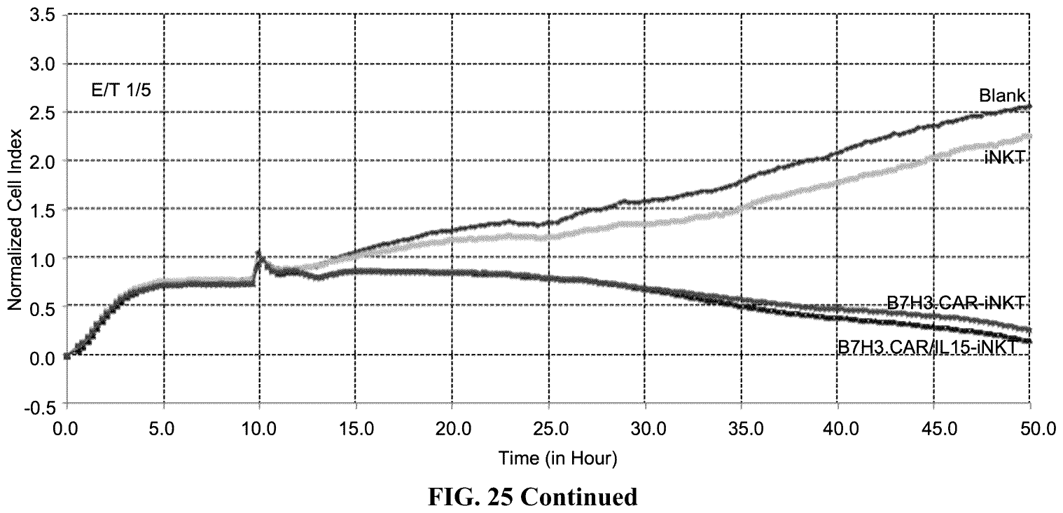

The results of the experiment are shown in FIG. 25, and the results in FIG. 25 show that: the B7H3.CAR/IL15-iNKT cells showed specific killing activity, the killing activity of the B7H3.CAR/IL15-iNKT and the B7H3.CAR-iNKT cells under the condition of the same effector-to-target ratio had significant difference, and the specific killing activity of the B7H3.CAR/IL15-iNKT was significantly higher than that of the B7H3.CAR-iNKT.

### Example 14. Validation of In Vivo Clearance of Ovarian Cancer Intraperitoneally Transplanted Tumor by the Fully Human B7H3.CAR-iNKT Cell

### 1. Method

6 weeks old male NCG mice were purchased. A mouse ovarian cancer model was constructed by a tail vein injection of 5×10⁵ SKOV-3-Ffluc-GFP. and the condition of the tumor formation was monitored using small animal in vivo imaging; after 5 days, the mice were randomly grouped into Ctrl group, B7H3.CAR-iNKT, and B7H3.CAR/IL15-iNKT, with 5 mice in each group, and 3 groups in total; on day 0, treatment was performed by tail vein infusion of B7H3.CAR-iNKT and B7H3.CAR/IL15-iNKT cells, 5×10⁶/mice; only PBS was infused in Ctrl group; the effect of treatment was observed using small animal in vivo imaging on days 3, 7, 21, and 35 after treatment, and survival of CAR-iNKT in vivo was detected by blood sampling via submaxillary vein.

### 2. Results

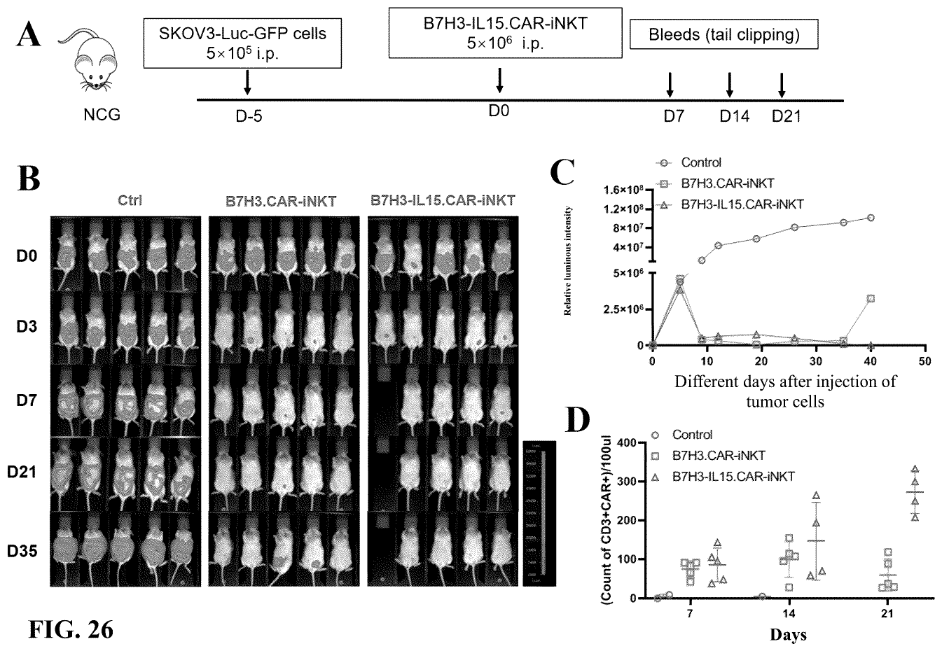

The results of the experiment are shown in FIG. 26, and the results of FIG. 26 panel A show: establishment of NCG mouse ovarian cancer intraperitoneally transplanted tumor model and treatment pattern diagram using B7H3.CAR-iNKT cells; the results of FIG. 26 panel B show that: 3 days after treatment, the tumor of the mice in two treatment groups began to regress, and no tumor survived within 1 month after treatment, but after 35 days, the mice in the B7H3.CAR-iNKT group recurred, while the tumor of the mice in the B7H3.CAR/IL15-iNKT group remained completely regressed; the results of FIG. 26 panel C show: BLI signal intensity of in vivo imaging of the mice in each group after treatment; the results of FIG. 26 panel D show that: CAR-iNKT cell content in peripheral blood of the mice in the B7H3.CAR/IL15-iNKT group was 10 times than that of the control group, indicating that IL15 could promote CAR-iNKT cell survival in vivo.

### Example 15. Preparation of the Fully Human B7H3.CAR/IL-21-INKT (B7H3-02) Targeting B7H3 and Co-Expressing IL-21

### 1. Method

(1) Preparation of iNKT
1) isolation of PBMCs: peripheral blood of a donor was collected, and the whole blood was diluted with an equal amount of normal saline. A lymphocyte isolation solution and the diluted blood were added into a centrifuge tube according to the ratio of 1:2 and centrifuged at 2000 rpm/min for 20 min. The cells in the buffy coat layer were collected, washed twice with normal saline, and centrifuged at 1500 rpm/min for 8 min to give peripheral blood mononuclear cells PBMCs;
2) induction of iNKT cells: the PBMCs were resuspended with lymphocyte culture medium and adjusted to a concentration of 2×10⁶/mL. α-Galcer, IL-2, IL-21, IL-4, and GM-CSF were added. The cells were inoculated onto a 24-well plate and placed in an incubator at 37° C. with 5% CO₂. The cell state was observed every day, and half of the medium was exchanged every other day;
3) magnetic bead sorting iNKT cells: the induced cells were collected on day 10 and resuspended using 500 μL of MACS buffer. Anti-iNKT MicroBeads were added according to the amount in the instruction, mixed well, placed at 4° C., and incubated for 30 min. 5 mL of MACS buffer was added for washing, the mixture was centrifuged at 400×g for 5 min, and the supernatant was discarded; the cells were resuspended using 500 μL of MACS buffer, loaded onto a LS sorting column, washed 3 times by MACS buffer, 3 mL each time; finally, the sorting column was placed in a collecting tube, and 500 μL of MACS buffer was added for elution to give iNKT positive cells;
4) activation and amplification of iNKT cells: the resulting cells in the previous purification step were resuspended using lymphocyte culture medium containing IL-7 and IL-15 on day 10, inoculated on CD3Ab and CD28Ab pre-coated plates, and placed in an incubator at 37° C. with 5% CO₂ for extensive amplification.

### (2) Construction of CAR Expression Vector

1) an scFv encoding sequence targeting human B7H3 was synthesized, and the scFv comprises a heavy chain VH and a light chain VL linked by a G4S short peptide;
2) a retroviral vector MSCV and the synthesized scFv targeting human B7H3 were digested with Nco I and Mlu I, the fragments were recovered, and the recovered target fragments were connected by T4 ligase and then transformed into Stb13 competent cells;
3) a single clone was selected for plasmid extraction, and after enzyme digestion and identification, it was sent for sequencing confirmation. The correct plasmid was B7H3.CAR;

In the construction method described above, the amino acid sequence of the fully human chimeric antigen receptor targeting B7H3 and co-expressing IL-21 is set forth in SEQ ID NO: 60, and the nucleotide sequence is set forth in SEQ ID NO: 61.

(3) Retrovirus Packaging 6 μg of a shuttle plasmid MSCV-B7H3.CAR comprising a CAR structure and 4 μg of a helper plasmid pCL-Ampho were mixed in 300 μL of opti-MEM culture medium. 30 μL of Genejuice transfection reagent was added dropwise in another 300 μL of opti-MEM culture medium, gently mixed well, and left to stand at room temperature for 5 min. The mixture containing the transfection reagent was added dropwise into the plasmid mixture, mixed well by shaking, and left to stand at room temperature for 15 min. Then PEI and the plasmid mixture were added dropwise into pre-plated 293T cell culture dish, and mixed well by gentle shaking. After 48-72 h, the supernatant was collected, filtered through a 0.45 μm syringe filter, and stored in an ultra-low temperature refrigerator for later use.
(4) Viral Infection of iNKT Cells

B7H3.CAR virus liquid was added into 10 μM HEPES and 6-8 μg/mL polybrene, and mixed well. Activated iNKT cells were resuspended using the virus liquid, then added into a 24-well plate pre-coated with RetroNectin, and centrifuged at 1500 g at 30° C. for 2 h. Then the supernatant was removed. X-Vivo culture medium containing 5% fetal bovine serum, 200 U/mL IL-2, 10 ng/ml IL-7, and 5 ng/ml IL-15 was supplemented. The amplification and culture was continued to give the fully human B7H3.CAR-iNKT cell B7H3.CAR/IL-21-iNKT targeting B7H3 and co-expressing IL-21.

### (5) Detection of CAR Expression Efficiency

After 48-72 h of virus infection, 2×10⁵ cells were taken for staining. 1 μg/mL of B7H3-Fc protein (R&D, 1027-B3-100) was firstly added and incubated at 4° C. for 30 min. The cells were washed and then the AF647-anti-human IgG antibody (Jackson, 109-606-088) was added and incubated in the dark at 4° C. for 30 min. The cells were washed and finally PerCP/Cy5.5-CD3 (Biolegend, 317336), PE-iNKT (BD, #552825), and an antibody were added and incubated in the dark at 4° C. for 30 min. After washing, the mixture was loaded on the machine for assay.

### 2. Results

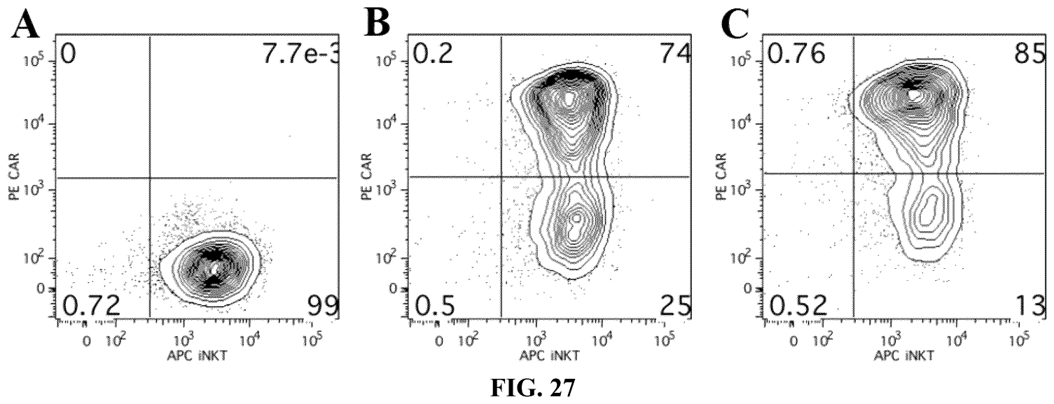

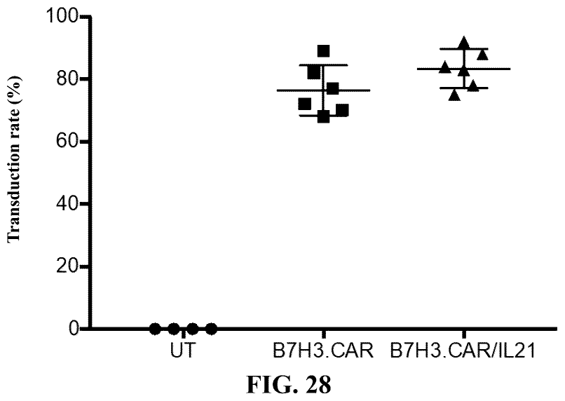

The results of CAR transfection efficiency of the B7H3.CAR/IL-21-iNKT cells prepared above assayed by flow cytometry are shown in FIG. 27 and FIG. 28, and the results show that: the CAR transfection efficiency of the B7H3.CAR/IL-21-iNKT cells was up to 75-92%, which was significantly higher than that of the B7H3.CAR-iNKT and iNKT, indicating that the CAR transfection efficiency of the B7H3.CAR/IL-21-iNKT cells prepared by the present application was high.

### Example 16. Detection of Cytokine Release Capacity and Apoptosis of the Fully Human B7H3.CAR/IL-21-INKT Targeting B7H3 and Co-Expressing IL-21

### 1. Method

### (1) Detection of Cytokine Release Ability Using ELISA Kit

2×10⁵ B7H3.CAR/IL-21-iNKT cells were collected, respectively mixed well with 2×10⁵ kidney cancer cells 786-O and OSRC-2, and added to a 24-well plate for co-incubation. A replicate well was set. After 24 h, the culture supernatant was collected; the content of IFN-γ and IL-2 was detected using an ELISA kit.

### (2) Detection of Apoptosis of the B7H3.CAR-iNKT Cell Using Flow Cytometer

The B7H3.CAR-iNKT and B7H3.CAR/IL-21-iNKT cells prepared in this example were collected, resuspended in T cell culture medium without cytokines (IL-2/IL-7/IL-15), and placed in a CO₂ incubator. The washed cells were collected at 0 h and 72 h, respectively, resuspended with 1× Annexin V Binding Buffer, added with FITC-Annexin V and PI, incubated in the dark at room temperature for 15 min, washed, resuspended, and loaded on the machine for assay. The effect of co-expression of IL-21 on apoptosis of B7H3.CAR-iNKT cells was analyzed.

### 2. Results

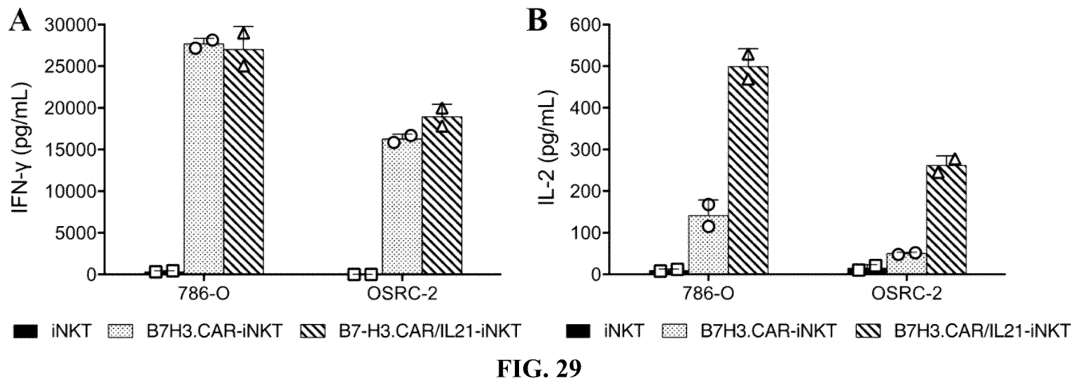

The results of the experiment are shown in FIG. 29, and the results show that: compared with the iNKT and the B7H3.CAR-iNKT, the B7H3.CAR/IL-21-iNKT cells prepared by the present application could secrete higher-level cytokines IL-2, and the secretion of the cytokine IFN-γ had no significant difference, indicating that the B7H3.CAR/IL-21-iNKT cells prepared by the present application had stronger amplification capacity and survival capacity.

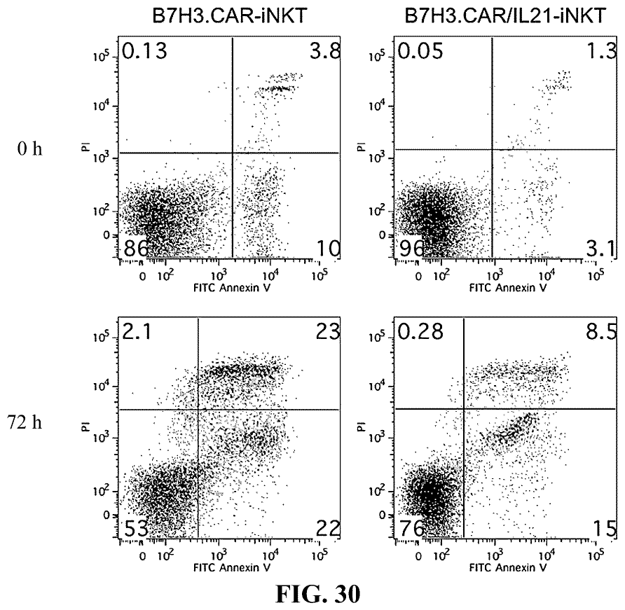

The results of FIG. 30 show that: 72 h after starvation culture, the B7H3.CAR-iNKT cells were subjected to massive apoptosis, and the apoptosis proportion of the B7H3.CAR/IL-21-iNKT cells was significantly less than that of the B7H3.CAR-iNKT cells, indicating that the anti-apoptosis capacity of the B7H3.CAR/IL21-iNKT cells prepared by the present application was enhanced.

### Example 17. Validation of the Ability of the B7H3.CAR/IL-21-INKT Cell to Kill Kidney Cancer Cell In Vitro

### 1. Method

Firstly, 50 μL DMEM complete culture medium was added to an E-Plate detection plate of an xCELLigence cell function analyzer, and the background impedance value was measured; kidney cancer cells 786-O and OSRC-2 in logarithmic phase were collected, the concentration of the cell suspension was adjusted to 1×10⁵/mL, 100 μL of the cell suspension was added to the E-Plate detection plate, and the plate was left to stand at room temperature for 30 min and placed on a detection platform; real-time dynamical observing was performed, when the proliferation of the target cells was in a plateau, 50 μL of effector cells iNKT, B7H3.CAR-iNKT, and B7H3.CAR/IL-21-iNKT were added to the experimental well according to effector-to-target ratios of 5/1, 1/1, and ⅕, and individual tumor cells were set as a control group; cell-mediated cell killing effect curve was observed in real time.

### 2. Results

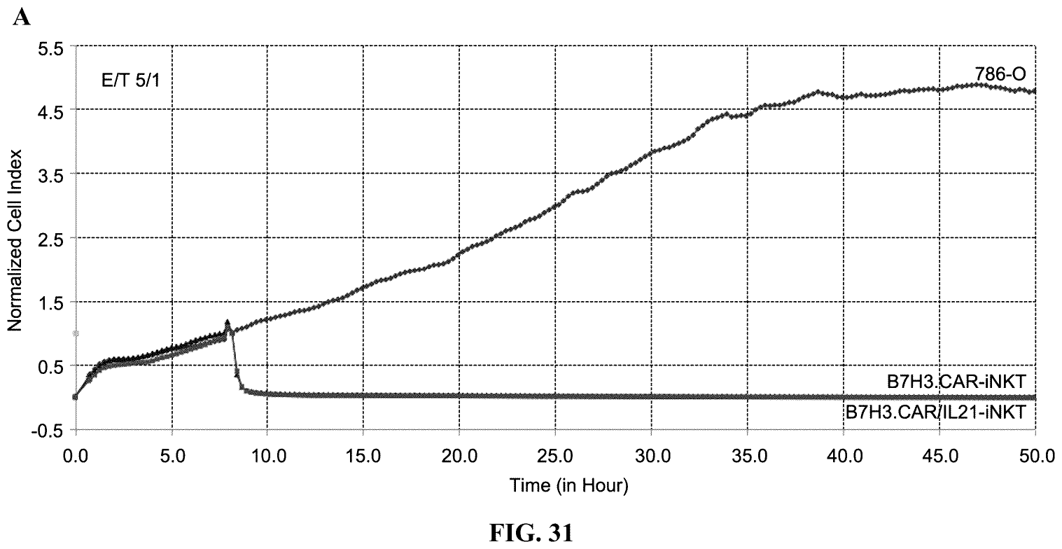

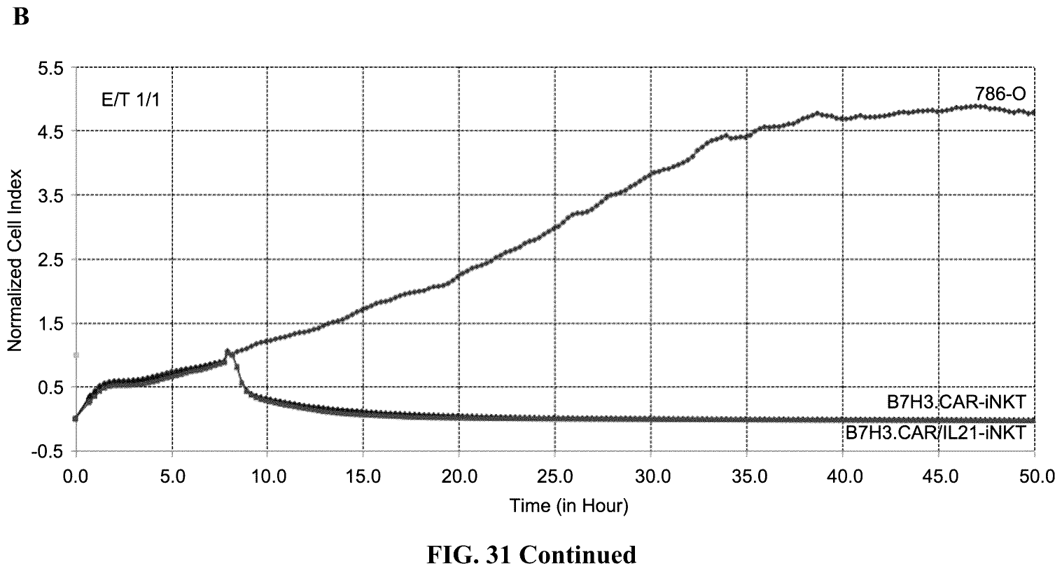

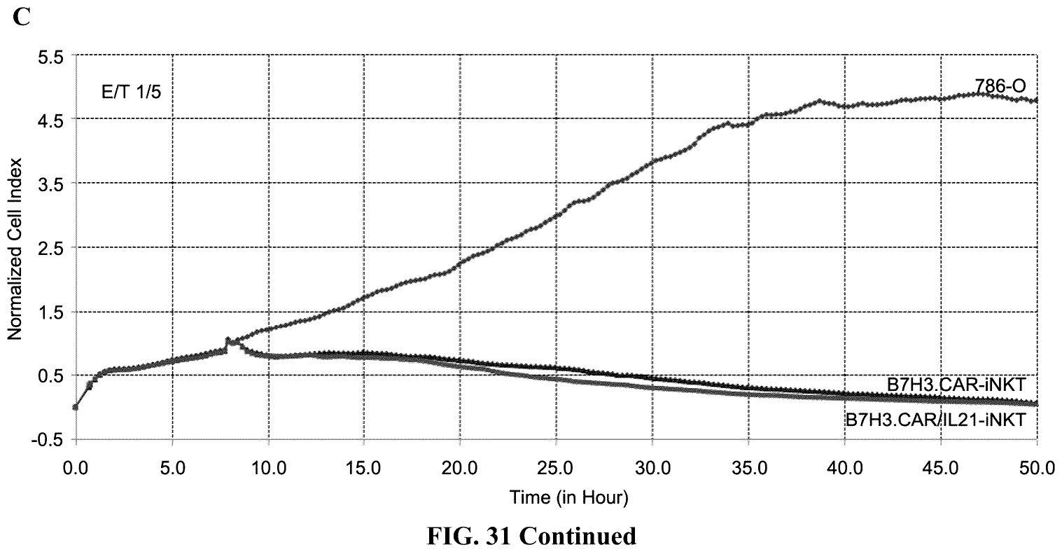

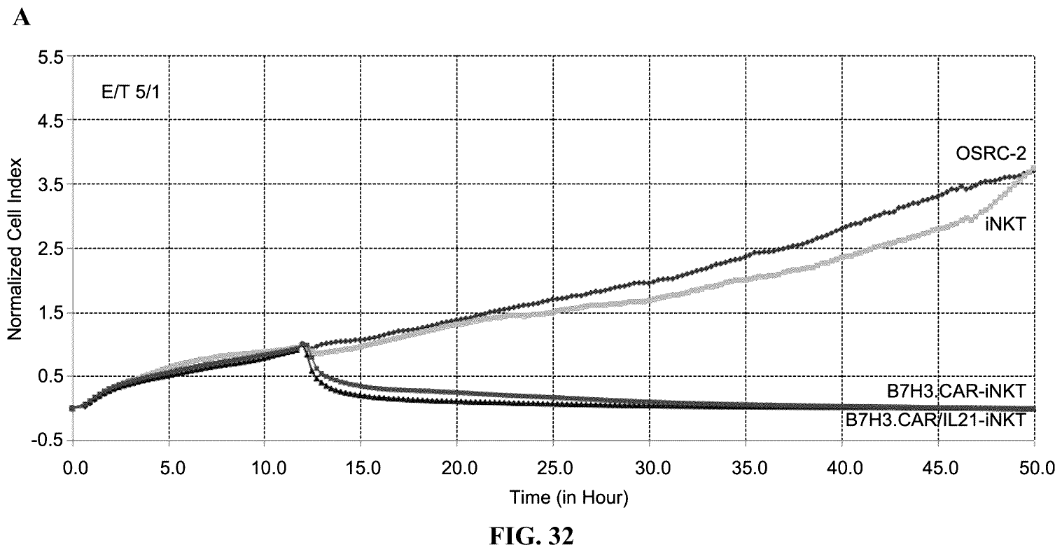

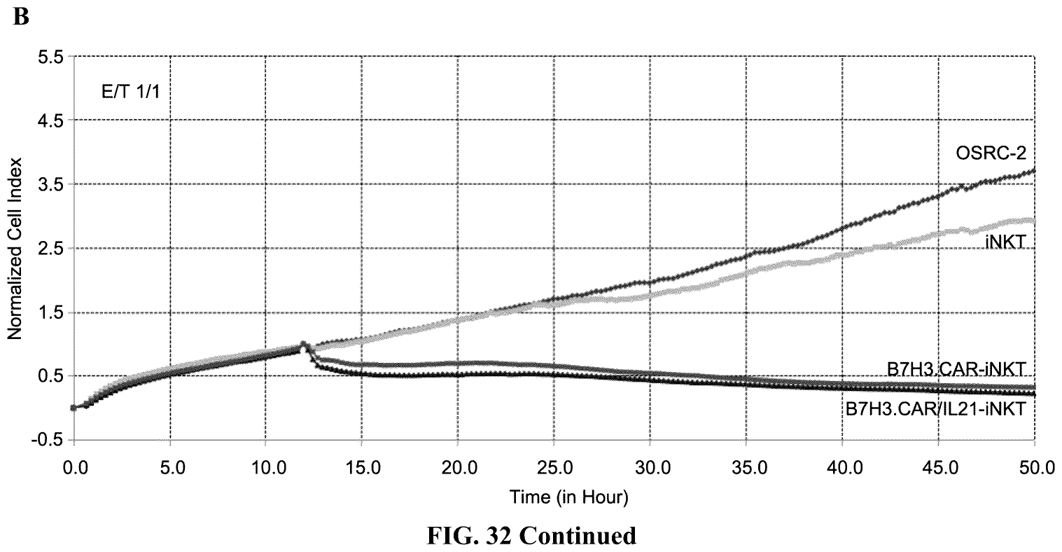

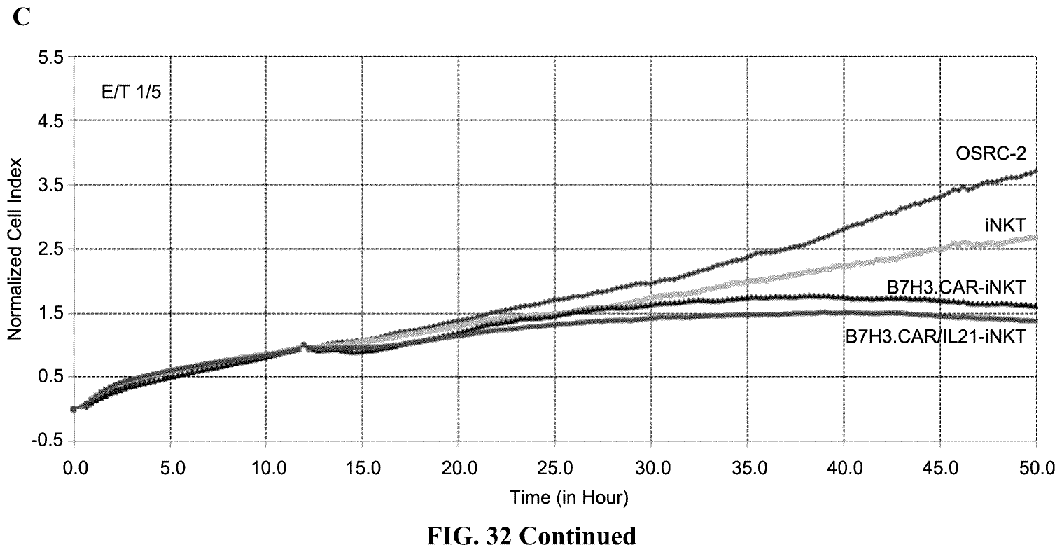

The results of the experiment are shown in FIGS. 31-32, and the results show that: both the B7H3.CAR/IL-21-iNKT and B7H3.CAR-iNKT cells could efficiently kill tumor target cells with high expression of B7H3, and the in vitro killing activities of the two had no significant difference.

### Example 18. Validation of In Vivo Clearance of Kidney Cancer Transplanted Tumor by the B7H3.CAR/IL-21-iNKT Cell

### 1. Method

6 weeks old male NCG mice were purchased. A mouse kidney cancer subcutaneously transplanted tumor model was constructed by a subcutaneous injection of 4×10⁶ 786-O-Luc-GFP cells. On day 10, after the tumor was formed, the mice were randomly grouped into Blank group, B7H3.CAR-iNKT group, and B7H3.CAR/IL-21-iNKT group, with 5 mice in each group, and 3 groups in total; on days 11 and 18, treatment was performed by tail vein infusion of B7H3.CAR-iNKT and B7H3.CAR/IL-21-iNKT cells, respectively, 5×10⁶/mice; treatment effect was observed twice a week by measuring tumor volume, survival of CAR-iNKT in vivo was detected by blood sampling via submaxillary vein, and survival of the mice was recorded.

### 2. Results

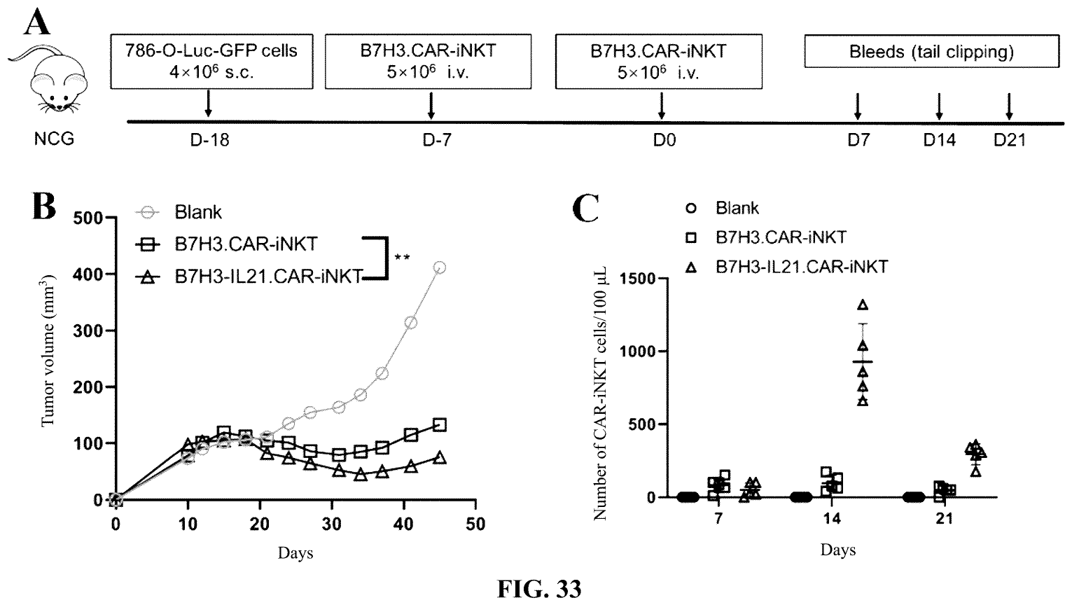

The results of the experiment are shown in FIG. 33, and the results of FIG. 33 panel A show: establishment of NCG mouse kidney cancer subcutaneously transplanted tumor model and treatment pattern diagram using B7H3.CAR-iNKT and B7H3.CAR/IL-21-iNKT cells; the results of FIG. 33 panel B show that: compared with Blank group and B7H3.CAR-iNKT group, the B7H3.CAR/IL-21-INKT cells had better ability to inhibit tumor growth; the results of FIG. 33 panel C show that: 14 and 21 days after treatment, the number of the CAR-iNKT cells in peripheral blood of the mice in B7H3.CAR/IL-21-iNKT group was significantly higher than those in Blank group and B7H3.CAR-iNKT group, indicating that the in vivo survival ability of the B7H3.CAR/IL-21-iNKT cells was stronger.

### Example 19. Preparation of the Fully Human CD276 CAR-NK (B7H3-02) Cell

### I. Procedures

### 1. Preparation of Umbilical Cord Blood NK Cells;

1) isolation of CBMCs: umbilical cord blood was collected, and diluted with an equal amount of normal saline. A lymphocyte isolation solution and the diluted umbilical cord blood were added into a centrifuge tube according to the ratio of 1:2 and centrifuged at 2000 rpm/min for 20 min. The cells in the buffy coat layer were collected, washed twice with normal saline, and centrifuged at 1500 rpm/min for 8 min to give umbilical cord blood mononuclear cells CBMCs.
2) induction of NK cells: CBMCs cells were resuspended using NK cell culture medium (X-VIVO15+5% FBS+1% P/S+Glutamin), adjusted to a cell density of 1-2×10⁶/mL, transferred to a CD16 Ab pre-coated plate (added with 1 μg/mL CD16 Ab antibody solution, 4° C. overnight, the coating solution was discarded before use, washed twice with PBS); an activation factor combination was added: 50 ng/mL 4-1BBL, 0.01 KE/mL OK432, and 1000 U/mL IL-2, and placed in an incubator at 37° C. with 5% CO₂ for 3 days of incubation. The cells were collected by centrifugation, resuspended using fresh NK cell culture medium, added with 1000 U/mL IL-2, transferred to a common culture flask, and placed in an incubator at 37° C. with 5% CO₂ for 2 weeks of amplification. The cell state was observed daily and half of the medium was exchanged every other day.
3) NK purity detection: on days 7, 10, and 14 of culture, 2×10⁵ cells were taken, washed, added with Alexa Fluor488 CD3, APC CD56, and PerCP/Cy5.5 NKG2D antibodies, and incubated in the dark at 4 ºC for 30 min. After washing, the mixture was loaded on the machine for assay.

2. Construction of shuttle plasmid comprising CAR structure: the CAR structure has two types, one that comprises IL-15 and one that does not comprise IL-15, wherein the amino acid sequence of the CAR comprising IL-15 (comprising signal peptide, T2A, and IL-15) is set forth in SEQ ID NO: 62, and the nucleotide sequence is set forth in SEQ ID NO: 63; the above sequences were synthesized, connected to a retroviral vector MSCV, and then transformed into Stb13 competent cells; a single clone was selected for plasmid extraction, and after enzyme digestion and identification, it was sent for sequencing confirmation.

### 3. Virus Packaging

6 μg of a shuttle plasmid comprising a CAR structure and 4 μg of a helper plasmid pCL-Ampho were mixed in 300 μL of opti-MEM culture medium. 30 μL of PEI reagent was added dropwise in another 300 μL of opti-MEM culture medium, mixed well by shaking, and left to stand at room temperature for 5 min. The mixture containing the PEI reagent was added dropwise into the plasmid mixture, mixed well by shaking, and left to stand at room temperature for 15 min. Then PEI and the plasmid mixture were added dropwise into pre-plated 293T cell culture dish, and mixed well by gentle shaking. After 48-72 h, the supernatant was collected, filtered through a 0.45 μm syringe filter, and stored in an ultra-low temperature refrigerator for later use.

### 4. Viral Infection of NK Cells

CD276-CAR virus liquid was added into 10 μM HEPES and 6-8 μg/mL polybrene, and mixed well. Then activated NK cells were resuspended using the virus liquid, then added into a 24-well plate coated with RetroNectin, and centrifuged at 1500 g at 30° C. for 2 h. Then the supernatant was removed. X-Vivo culture medium containing 5% fetal bovine serum, 200 U/mL IL-2, 10 ng/ml IL-21, and 5 ng/ml IL-15 was supplemented. The culture and amplification was continued.

### 5. CAR-NK Cell Assay

After 72 h of virus infection, 2×10⁵ cells were taken for staining. 1 μg/mL of B7H3-Fc protein was firstly added and incubated at 4° C. for 30 min. The cells were washed and then the AF647-anti-human IgG Fc antibody was added and incubated in the dark at 4° C. for 30 min. The cells were washed and finally Alexa Fluor488 CD3 and APC CD56 antibodies were added and incubated in the dark at 4° C. for 30 min. After washing, the mixture was loaded on the machine for assay.

### II. Experimental Results

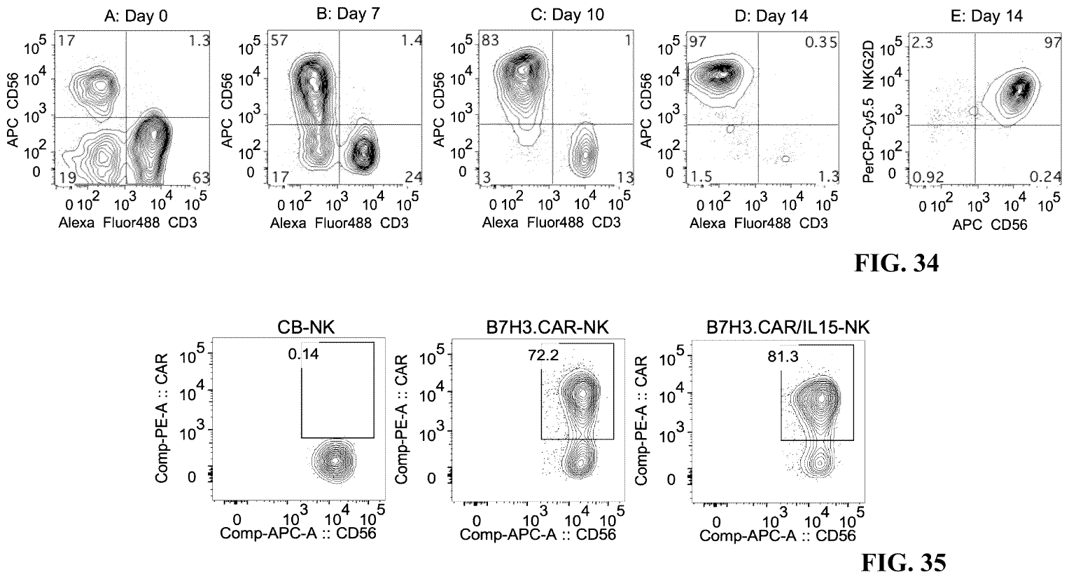

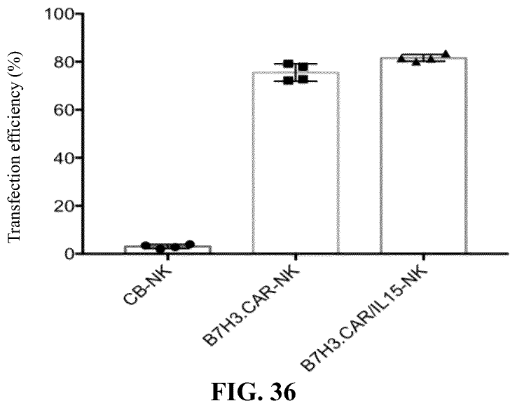

FIG. 34 is a flow cytometric representative plot of purity assay for NK cells cultured for different days, and the results show that NK cells prepared by the present application had a purity of greater than 95% and highly expressed NKG2D; FIG. 35 is a flow cytometric representative plot of CAR-NK transfection efficiency assay; FIG. 36 is a statistical graph of CAR-NK transfection efficiency, and the results show that induced NK cells could be infected with high efficiency using a retrovirus-mediated CAR system with CAR positive rate reaching 60-85%.

### Example 20. Detection of Killing Effect of CAR-NK Cell on Tumor Cell MCF-7 Using RTCA Real-Time Label-Free Dynamic Cell Analysis Technology

### I. Procedures

Firstly, 50 μL DMEM complete culture medium was added to an E-Plate detection plate of an xCELLigence cell function analyzer, and the background impedance value was measured; target cells MCF-7 in logarithmic phase were collected, the concentration of the cell suspension was adjusted to 1×10⁵/mL, 100 μL of the cell suspension was added to the E-Plate detection plate, and the plate was left to stand at room temperature for 30 min and placed on a detection platform; the proliferation state of the target cells was subjected to real-time dynamical observing, and after 24 h, 50 μL of effector cells were added to the experimental well according to effector-to-target ratios of 5:1, 2.5:1, and 1:1; individual tumor cells were set as Blank group, and the B7H3.CAR-NK cell-mediated killing effect curve was observed in real time.

### II. Results

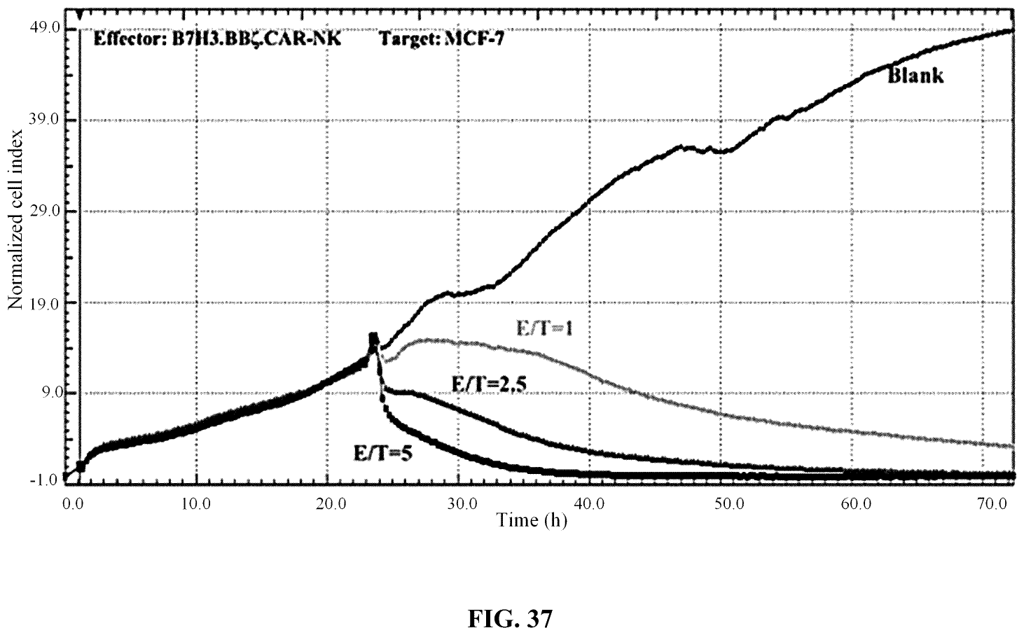

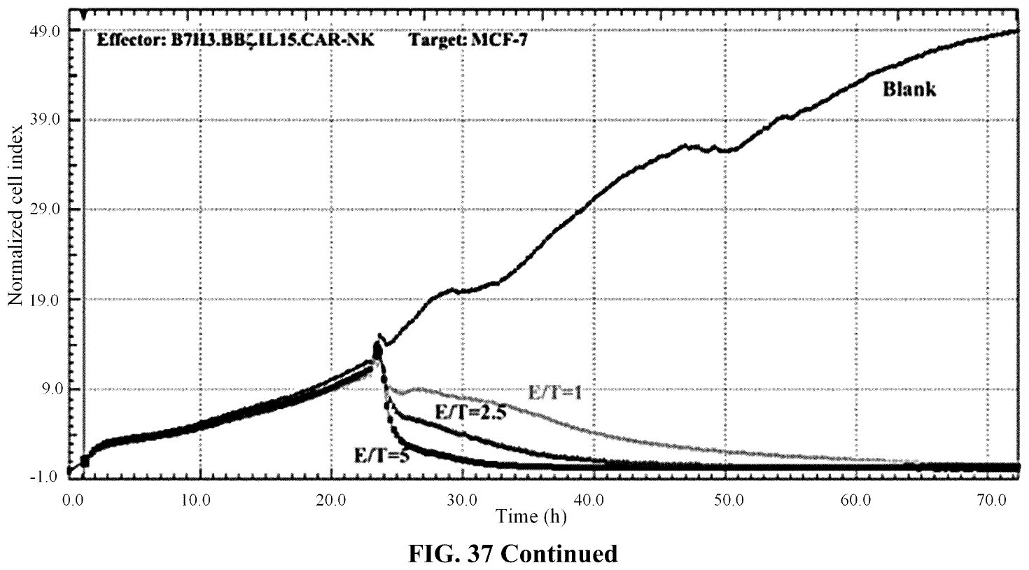

FIG. 37 is a dynamic curve of the killing of the CAR-NK cell on breast cancer cell MCF-7 analyzed by RTCA technology. The results show that the B7H3.CAR-NK (comprising signal peptide, T2A, and IL-15) cell prepared by the present application could kill the breast cancer cell MCF-7 with high efficiency, and the higher the effector-to-target ratio, the stronger the killing activity.

### Example 21. Preparation of the Fully Human CD276 CAR-T (CD276-01) Cell

### I. Procedures

### (1) Preparation of PBMC Cells

Peripheral blood of a healthy person was taken and centrifuged. The autologous plasma was reserved for later use. The remaining blood cells were diluted using an equal volume of normal saline, added to the upper layer of a lymphocyte isolation solution, and centrifuged. The cells in the middle buffy coat layer were pipetted, added with normal saline for washing, and centrifuged. The supernatant was discarded.

### (2) Construction of Shuttle Plasmid Comprising CAR Structure

a. the amino acid sequence of the synthetic CAR targeting human CD276 (comprising signal peptide, T2A, and tEGFR) is set forth in SEQ ID NO: 64, and the nucleotide sequence is set forth in SEQ ID NO: 65, wherein the amino acid sequence of the heavy chain VH of the scFv targeting human CD276 is set forth in SEQ ID NO: 8, the nucleotide sequence is set forth in SEQ ID NO: 10, the amino acid sequence of the light chain VL is set forth in SEQ ID NO: 18, and the nucleotide sequence is set forth in SEQ ID NO: 20; the amino acid sequence of the G4S short peptide is set forth in SEQ ID NO: 22, and the nucleotide sequence is set forth in SEQ ID NO: 24; the amino acid sequence of the CD276-01 scFv is set forth in SEQ ID NO: 26, and the nucleotide sequence is set forth in SEQ ID NO: 28.
b. a retroviral vector MSCV and the CAR encoding nucleotide sequence targeting human CD276 synthesized in step 1) were digested with Nco I and Mlu I, the fragments were recovered, and the recovered target fragments were connected by T4 ligase and then transformed into Stb13 competent cells;
c. a single clone was selected for plasmid extraction, and after enzyme digestion and identification, it was sent for sequencing confirmation. The correct plasmid was MSCV-M13B701.

### (3) Virus Packaging

6 μg of a shuttle plasmid comprising a CAR structure and 4 μg of a helper plasmid pCL-Ampho were mixed in 300 μL of opti-MEM culture medium. 30 μL of PEI reagent was added dropwise in another 300 μL of opti-MEM culture medium, mixed well by shaking, and left to stand at room temperature for 5 min. The mixture containing the PEI reagent was added dropwise into the plasmid mixture, mixed well by shaking, and left to stand at room temperature for 15 min. Then PEI and the plasmid mixture were added dropwise into pre-plated 293T cell culture dish, and mixed well by gentle shaking. After 48-72 h, the supernatant was collected, filtered through a 0.45 μm syringe filter, and stored in an ultra-low temperature refrigerator for later use.

### (4) Preparation of CAR-T Cell

a. Isolation of PBMC Cell

Peripheral blood of healthy volunteers was collected and centrifuged at 1300 g at room temperature for 10 min. Then the plasma part was discarded, and the remaining blood cells were diluted and mixed well using an equal volume of normal saline; the blood cell suspension was slowly added into the upper layer of a lymphocyte isolation solution, and centrifuged at 600 g at room temperature for 25 min; the lymphocytes in the middle buffy coat layer were pipetted and added with normal saline for washing, red blood cell lysis treatment was performed if necessary, the mixture was centrifuged at 400 g at room temperature for 10 min, and the supernatant was discarded to give PBMC cells.

b. Culture and Activation of PBMC Cell

A 24-well plate was firstly coated with 1 μg/mL anti-human CD3 (OKT3) and anti-human CD28 (CD28.2) and incubated overnight at 4° C.; PBMC cells were then resuspended to 1×10⁶/mL using X-Vivo culture medium containing 5% fetal bovine serum, 200 U/mL IL-2, 10 ng/ml IL-7, and 5 ng/ml IL-15, and 1 mL of the cell suspension was inoculated per well for culture and activation.

c. Infection of Activated PBMC Cell with CD276-CAR Virus

CD276-CAR virus liquid was added into 10 μM HEPES and 6-8 μg/mL polybrene, and mixed well. Then activated PBMC cells were resuspended using the virus liquid, then added into a 24-well plate coated with RetroNectin, and centrifuged at 1500 g at 30° C. for 2 h. Then the supernatant was removed. X-Vivo culture medium containing 5% fetal bovine serum, 200 U/mL IL-2, 10 ng/ml IL-7, and 5 ng/mL IL-15 was supplemented. The culture was continued.

### (5) Detection of Infection Efficiency in CAR-T Cell

The expression of CD276-CAR in CAR-T was detected using flow cytometer, and the infection efficiency was analyzed.

### (6) Detection of Proliferation Capacity of CAR-T Cell

The number of CAR-T cells cultured for different days was determined, and the growth curve was plotted.

### II. Experimental Results

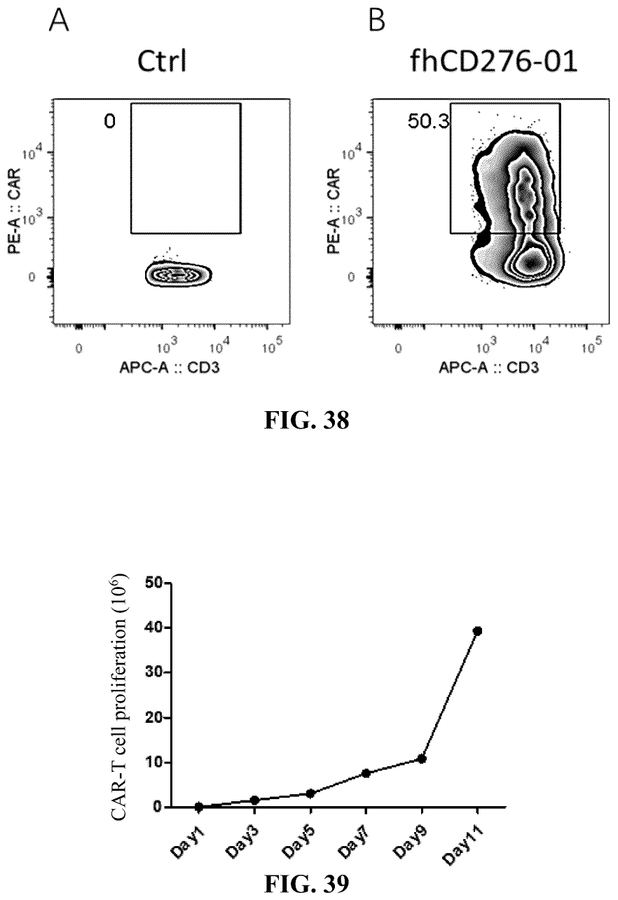

The results are shown in FIG. 38. CAR-T of the present application could efficiently express CAR targeting CD276 with high infection efficiency.

The results are shown in FIG. 39. CAR-T cells of the present application proliferated rapidly.

### Example 22. In Vitro Functional Validation of the Fully Human CD276 CAR-T Cell

### I. Procedures

50 μL of cytokine-free T cell complete culture medium (without cytokine) was added to an E-Plate detection plate, and the background impedance value was measured. 1×10⁴ tumor cells (tumor cells/100 μL) were added to the E-Plate detection plate. The cells were observed. When the tumor cells adhered to the wall, CAR-T cells were added to the E-Plate detection plate according to effector-to-target ratios (E/T) of 2:1, 1:1, and 1:2, and the system 200 μL was balanced using a culture medium and placed on a detection platform (the detection platform was put into an incubator in advance). A real-time dynamic cell proliferation detection was performed.

### II. Experimental Results

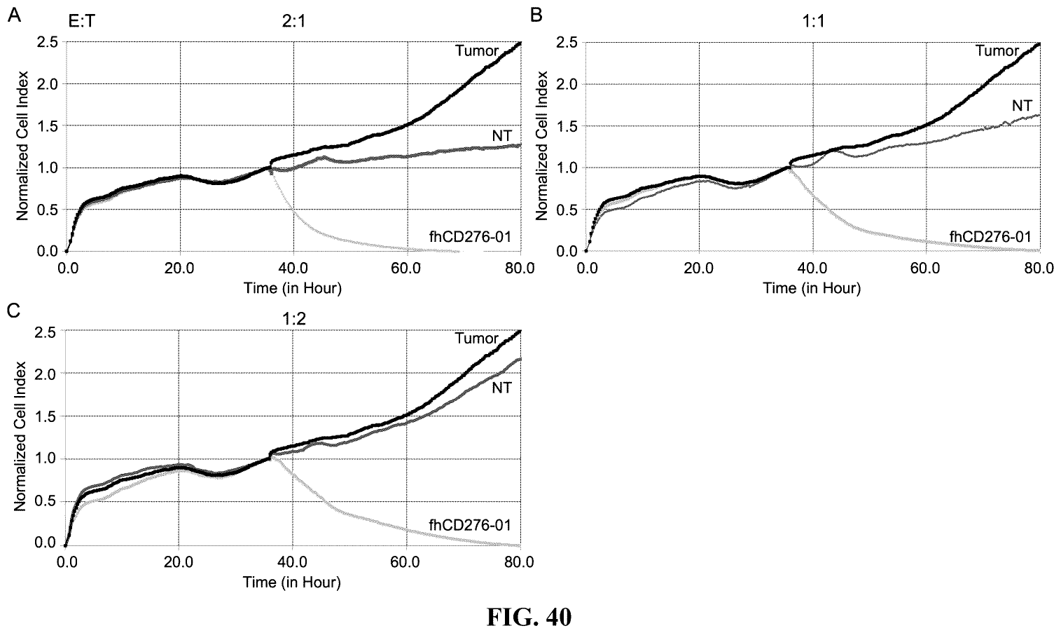

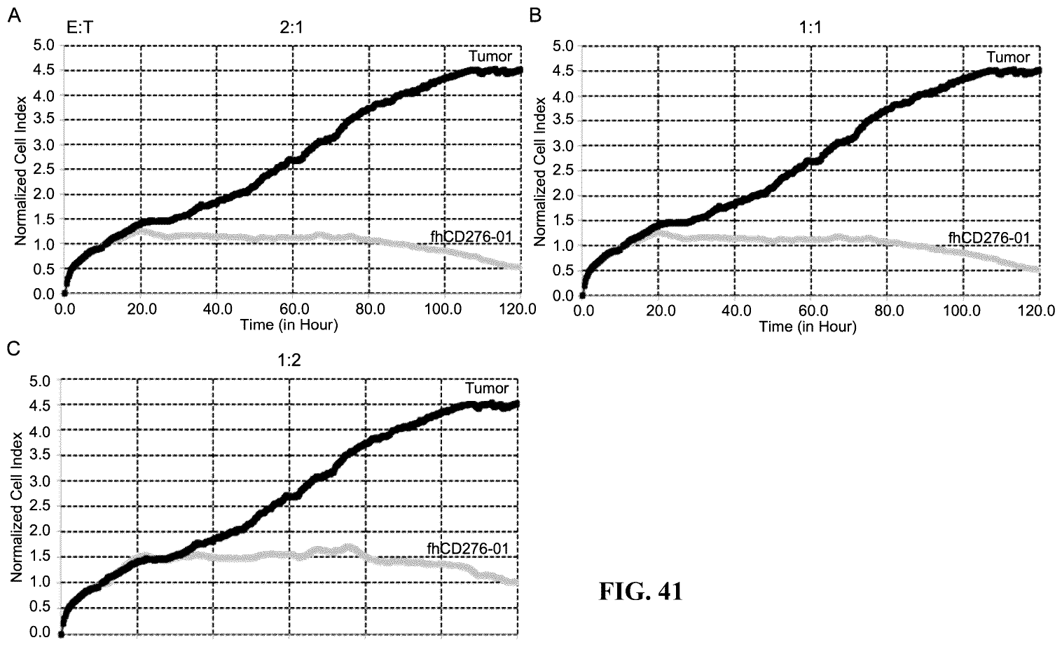

The results are shown in FIGS. 40 and 41. The CAR-T cells of the present application could kill tumor cells efficiently in vitro.

### Example 23. Preparation of the Fully Human CD276 CAR-T (B7H3-02) Cell

### I. Procedures

### (1) Preparation of PBMC Cells

Peripheral blood of a healthy person was taken and centrifuged. The autologous plasma was reserved for later use. The remaining blood cells were diluted using an equal volume of normal saline, added to the upper layer of a lymphocyte isolation solution, and centrifuged. The cells in the middle buffy coat layer were pipetted, added with normal saline for washing, and centrifuged. The supernatant was discarded.

### (2) Construction of Shuttle Plasmid MSCV-M13B702 Comprising CAR Structure

a. the amino acid sequence of the synthetic CAR targeting human CD276 (comprising signal peptide, T2A, and tEGFR) is set forth in SEQ ID NO: 66, and the nucleotide sequence is set forth in SEQ ID NO: 67;
b. a retroviral vector MSCV and the CAR encoding nucleotide sequence targeting human CD276 synthesized in step 1) were digested with Nco I and Mlu I, the fragments were recovered, and the recovered target fragments were connected by T4 ligase and then transformed into Stb13 competent cells;
c. a single clone was selected for plasmid extraction, and after enzyme digestion and identification, it was sent for sequencing confirmation. The correct plasmid was MSCV-M13B702.

### (3) Virus Packaging

6 μg of the shuttle plasmid MSCV-M13B702 comprising a CAR structure and 4 μg of a helper plasmid pCL-Ampho were mixed in 300 μL of opti-MEM culture medium. 30 μL of PEI reagent was added dropwise in another 300 μL of opti-MEM culture medium, mixed well by shaking, and left to stand at room temperature for 5 min. The mixture containing the PEI reagent was added dropwise into the plasmid mixture, mixed well by shaking, and left to stand at room temperature for 15 min. Then PEI and the plasmid mixture were added dropwise into pre-plated 293T cell culture dish, and mixed well by gentle shaking. After 48-72 h, the supernatant was collected, filtered through a 0.45 μm syringe filter, and stored in an ultra-low temperature refrigerator for later use.

### (4) Preparation of CAR-T Cell

a. Isolation of PBMC Cell

Peripheral blood of healthy volunteers was collected and centrifuged at 1300 g at room temperature for 10 min. Then the plasma part was discarded, and the remaining blood cells were diluted and mixed well using an equal volume of normal saline; the blood cell suspension was slowly added into the upper layer of a lymphocyte isolation solution, and centrifuged at 600 g at room temperature for 25 min; the lymphocytes in the middle buffy coat layer were pipetted and added with normal saline for washing, red blood cell lysis treatment was performed if necessary, the mixture was centrifuged at 400 g at room temperature for 10 min, and the supernatant was discarded to give PBMC cells.

b. Culture and Activation of PBMC Cell

A 24-well plate was firstly coated with 1 μg/mL anti-human CD3 (OKT3) and anti-human CD28 (CD28.2) and incubated overnight at 4° C.; PBMC cells were then resuspended to 1×10⁶/mL using X-Vivo culture medium containing 5% fetal bovine serum, 200 U/mL IL-2, 10 ng/ml IL-7, and 5 ng/ml IL-15, and 1 mL of the cell suspension was inoculated per well for culture and activation.

c. Infection of Activated PBMC Cell with CD276-CAR Virus

CD276-CAR virus liquid was added into 10 μM HEPES and 6-8 μg/mL polybrene, and mixed well. Then activated PBMC cells were resuspended using the virus liquid, then added into a 24-well plate coated with RetroNectin, and centrifuged at 1500 g at 30° C. for 2 h. Then the supernatant was removed. X-Vivo culture medium containing 5% fetal bovine serum, 200 U/mL IL-2, 10 ng/ml IL-7, and 5 ng/ml IL-15 was supplemented. The culture was continued.

### (5) Detection of Infection Efficiency in CAR-T Cell

The expression of CD276-CAR in CAR-T was detected using flow cytometer, and the infection efficiency was analyzed.

### (6) Detection of Proliferation Capacity of CAR-T Cell

The number of CAR-T cells cultured for different days was determined, and the growth curve was plotted.

### II. Experimental Results

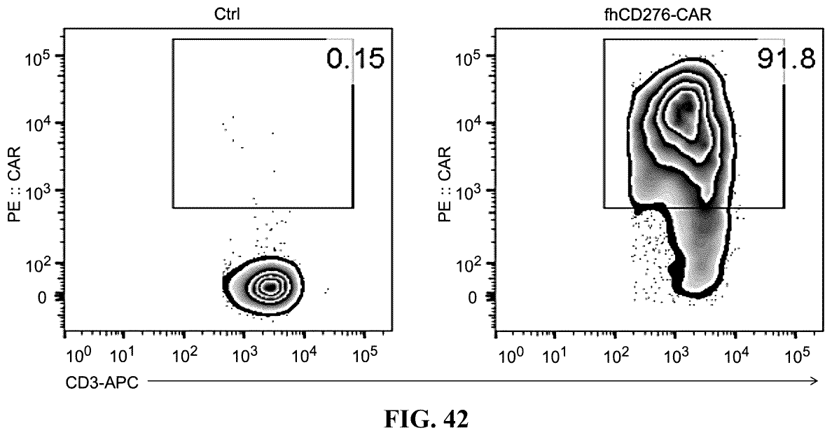

The results are shown in FIG. 42. CAR-T of the present application could efficiently express CAR targeting CD276 with high infection efficiency.

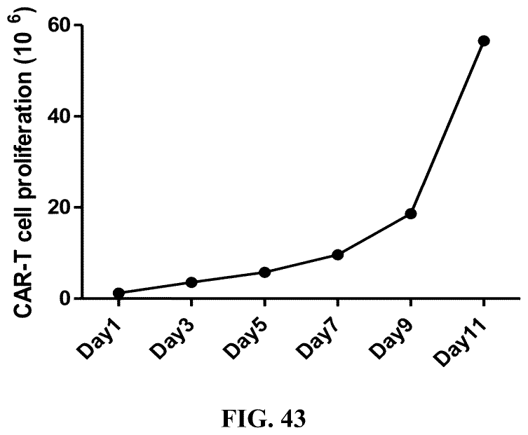

The results are shown in FIG. 43. CAR-T cells of the present application proliferated rapidly.

### Example 24. In Vitro Functional Validation of the Fully Human CD276 CAR-T (B7H3-02) Cell

### I. Procedures

50 μL of cytokine-free T cell complete culture medium (without cytokine) was added to an E-Plate detection plate, and the background impedance value was measured. 1×10⁴ tumor cells (tumor cells/100 μL) were added to the E-Plate detection plate. The cells were observed. When the tumor cells adhered to the wall, CAR-T cells were added to the E-Plate detection plate according to effector-to-target ratios (E/T) of 2:1, 1:1, and 1:2, and the system 200 μL was balanced using a culture medium and placed on a detection platform (the detection platform was put into an incubator in advance). A real-time dynamic cell proliferation detection was performed.

### II. Experimental Results

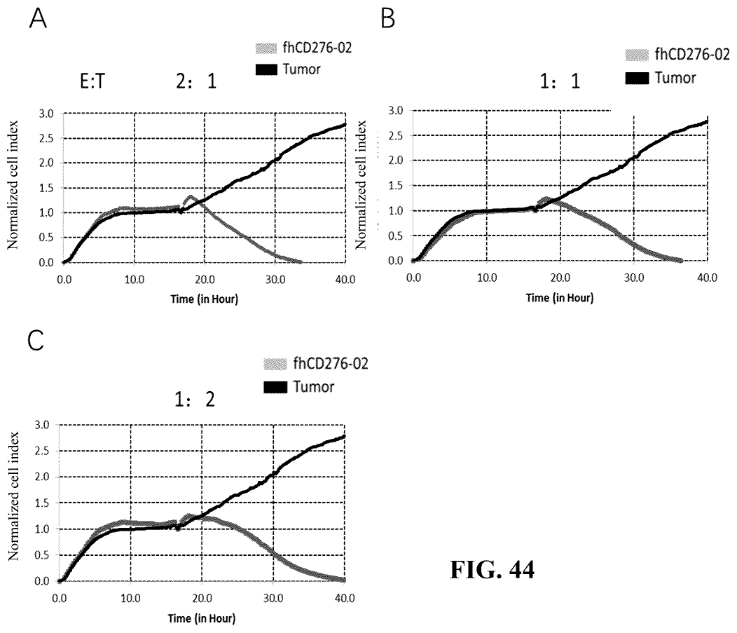

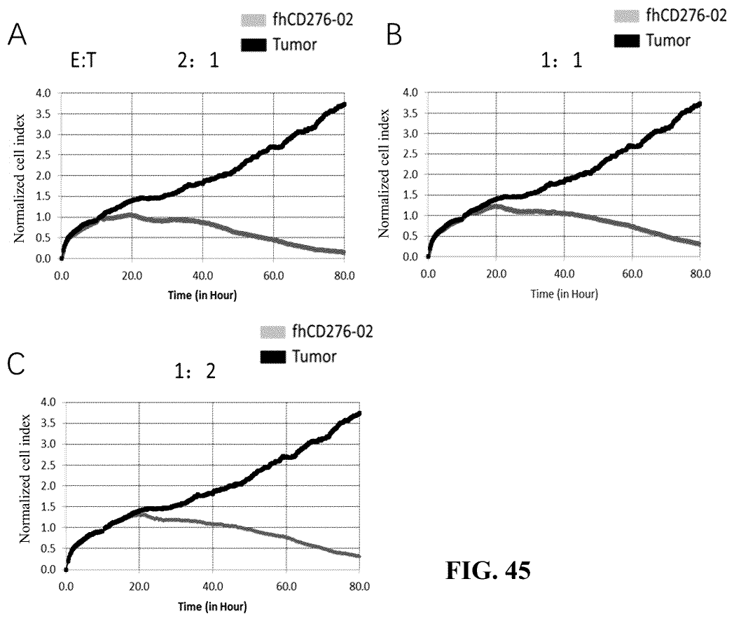

The results are shown in FIGS. 44 and 45. The CAR-T cells of the present application could kill tumor cells efficiently in vitro.

### Example 25. In Vivo Functional Validation of the Fully Human CD276 CAR-T (B7H3-02) Cell

### I. Procedures

5×10⁵ SKOV3-luc-GFP carrying a fluorescence signal was intraperitoneally injected into NCG mice. The mice were monitored for the condition of the formation of a peritoneal tumor by weekly photography using small animal in vivo imaging. The fully human fhCD276-02 CAR-T cells were intraperitoneally injected after the formation of a peritoneal tumor. Humanized CD276 CAR-T cells were used in the control group. The mice were then observed weekly for the condition of the regression of the peritoneal tumor by in vivo imaging.

### II. Experimental Results

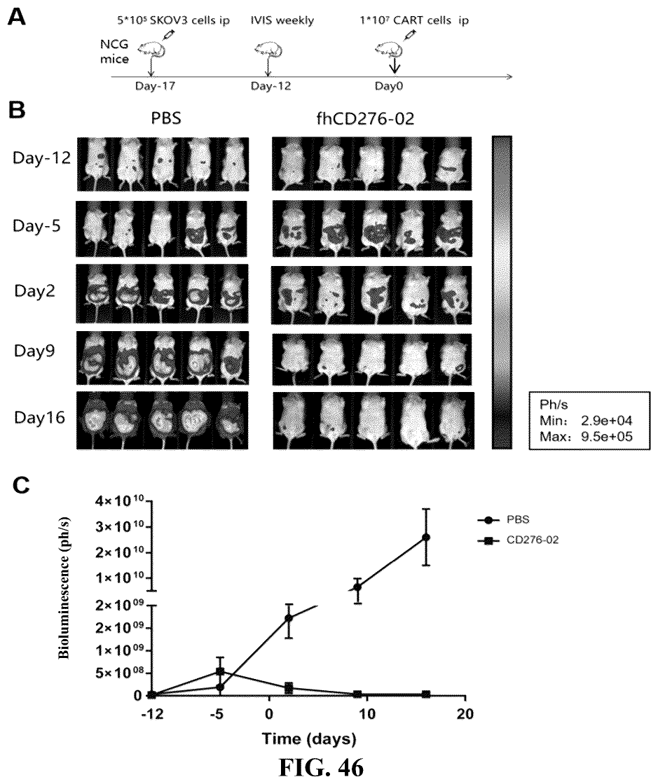

The results are shown in FIG. 46. CAR-T cells of the present application could clear mouse peritoneal ovarian cancer transplanted tumors.

### Example 26. Preparation of B7H3-CAR-T (B7H3-02) with High Expression of hGSTP1

### 1. Method

### (1) Isolation of PBMC Cell

1) peripheral blood of healthy volunteers was collected and centrifuged at 1300 g at room temperature for 10 min. Then the plasma part was discarded, and the remaining blood cells were diluted and mixed well using an equal volume of normal saline;
2) the blood cell suspension was slowly added into the upper layer of a lymphocyte isolation solution, and centrifuged at 600 g at room temperature for 25 min;
3) the lymphocytes in the middle buffy coat layer were pipetted and added with normal saline for washing, red blood cell lysis treatment was performed if necessary, the mixture was centrifuged at 400 g at room temperature for 10 min, and the supernatant was discarded to give PBMC cells.

### (2) Construction of CAR Expression Vector

1) an scFv encoding sequence targeting human B7H3 was synthesized, and the scFv comprises a heavy chain VH and a light chain VL linked by a 3×G4S short peptide;
2) a retroviral vector MSCV and the scFv targeting human B7H3 synthesized in step 1) were digested with Nco I and Mlu I, the fragments were recovered, and the recovered target fragments were connected by T4 ligase and then transformed into Stb13 competent cells;
3) a single clone was selected for plasmid extraction, and after enzyme digestion and identification, it was sent for sequencing confirmation. The correct plasmid was MSCV-B7H3-Gstp1.

The amino acid sequence of the CAR (comprising signal peptide, T2A, and hGSTP1) obtained by the construction method described above is set forth in SEQ ID NO: 68, and the nucleotide sequence is set forth in SEQ ID NO: 69.

### (3) Retrovirus Packaging

1) 293T cells were prepared and plated at 3×10^{6/100} mm culture dish;
2) on the next day, 293T cell state was observed. The cells were in a good state, and transfection was performed;
3) the transfection reagent was prepared using a 1.5 mL EP tube: 30 μL Genejuice+470 μL IMDM, and incubated at room temperature for 5 min;
4) the shuttle plasmid MSCV-M13B702 and the helper plasmid pCL-Ampho were added to a new 1.5 mL EP tube according to the total amount of 10 μg and the ratio of 3:2, which was DNA Mix;
5) one part of the transfection reagent was added to the DNA Mix, gently mixed well, and incubated at room temperature for 15 min;
6) culture dishes were marked, the reagent obtained in the previous step was added to the culture dishes respectively, and the virus supernatant was collected after 48-72 h;
7) the supernatant was subpackaged in 1.5 mL EP tubes, each tube being 1 mL, and stored in a refrigerator at −80° C. for later use.

### (4) Retrovirus Transduction

1) Day −1: a 24-well plate was coated with hCD3/CD28 antibodies;
2) Day 0: human PBMCs were thawed, counted, and resuspended in L500 medium (L500+10% FBS+1% P.S., and cytokines 5 ng/mL IL-15 and 10 ng/ml IL-7 were added during CAR-T cell preparation) to 1×10⁶/mL. The coating solution was discarded. Each well was inoculated with 1 mL of cells;
3) Day 1: the 24-well plate was coated with 1 μg/mL Retronectin;
4) Day 2: after 48 h of cell activation, CAR virus infection was performed. The cells were collected into a centrifuge tube, counted, and distributed according to 0.5-1×10⁶ cells per tube. The tube was centrifuged, and the supernatant was discarded. T cells were resuspended with 1 mL of virus solution, and the T cells were inoculated onto the 24-well plate and centrifuged at 1500 g at 30° C. for 2 h. The supernatant was gently discarded, and L500 medium comprising cytokines was slowly added.
   (5) Amplification of B7H3-CAR-T Cell with High Expression of hGSTP1

Day 4-Day 14 the medium was supplemented to maintain the cell density at (0.5-1)×10⁶/mL depending on the cell growth and cell number.

### (6) Detection of CAR Expression Efficiency

Day 4: the T cell purity and the CAR positive rate were detected by flow cytometry. The cells were labeled by B7H3-Fc protein, incubated at room temperature for 20 min, and washed. Then the PE-Anti Human IgG-Fc antibody was added, incubated at room temperature for 20 min in the dark, and washed. Finally, APC-CD3 staining was performed, and analysis was performed using a flow cytometer.

### 2. Results

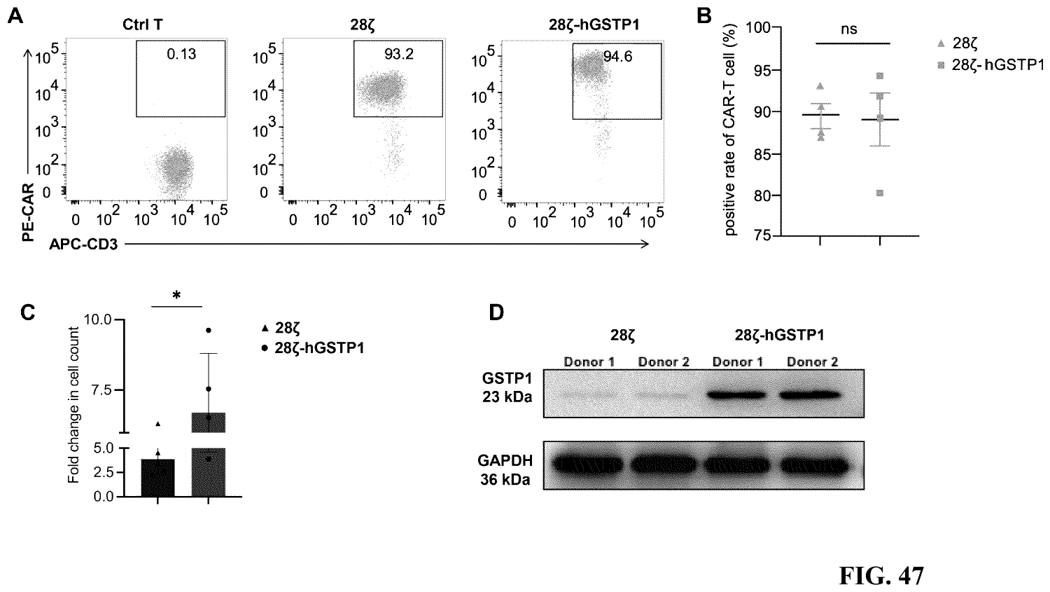

The results of the experiment are shown in FIG. 47, and the results show that the B7H3-CAR-T cell constructed by the present application comprises the GSTP1 gene, indicating that the present application successfully constructed a fully human CAR comprising the human GSTP1 gene targeting B7H3, and further prepared the B7H3-CAR-T cell. The results of the flow cytometer analysis show that the positive rate of CAR expression in the B7H3-CAR-T cells with high expression of GSTP1 was up to 90%. GSTP1 was efficiently expressed in the prepared CAR-T cells, and the expression of GSTP1 not only did not influence the positive rate of CAR expression, but also promoted the proliferation of B7H3-CAR-T cells.

### Example 27. B7H3-CAR-T Cell with High Expression of hGSTP1 Effectively Inhibiting Generation of Cellular Reactive Oxygen Species

### 1. Method

1) CAR-T was pipetted uniformly, collected into a sterile 1.5 mL EP tube, and centrifuged at 1800 rpm for 5 min. The supernatant was discarded.
2) the mixture was added with 1 mL PBS, washed once, and centrifuged at 1800 rpm for 5 min. The supernatant was discarded.
3) DCFH-DA working solution was prepared: 500 L PBS+0.5 L DCFH-DA.
4) 50-100 L of the DCFH-DA working solution was added into the EP tube, and incubated at 37° C. in the dark for 20-30 min.
5) the mixture was added with 1 mL PBS, washed 3 times, and centrifuged at 1800 rpm for 5 min. The supernatant was discarded.
6) 300 L PBS was added, and the cell pellet was resuspended, transferred into a flow cytometry tube, and loaded on the machine for assay.

### 2. Results

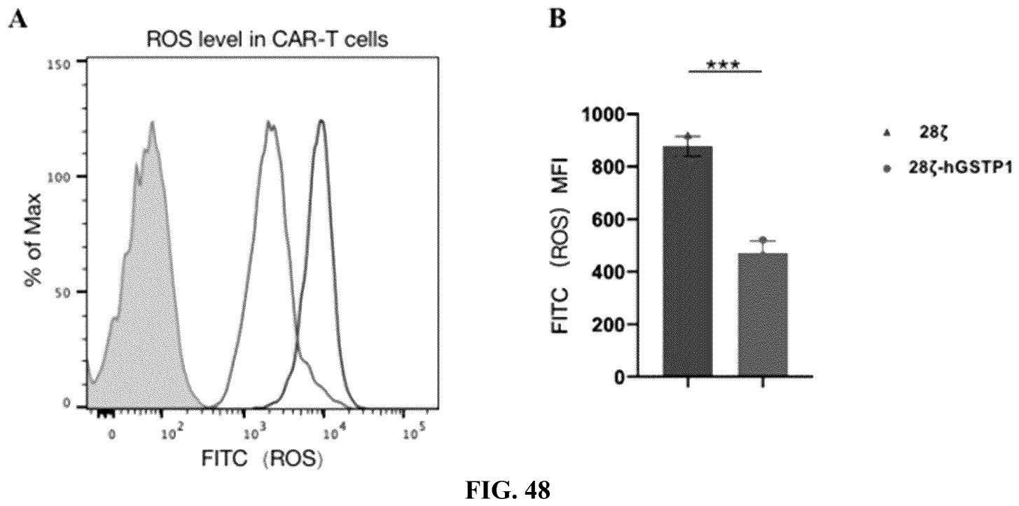

The results of the experiment are shown in FIG. 48. 282-CAR-T cells (representing CAR-T comprising a CD28 co-stimulatory domain) and 28ζ-bGSTP1 CAR-T cells (representing CAR-T comprising a CD28 co-stimulatory domain and expressing hGSTP1) were labeled with DCFH-DA, and the levels of reactive oxygen species of the CAR-T cells were measured by flow cytometry. The results show that relative to the control 28ζ-CAR-T, the reactive oxygen species level of the 28ζ-hGSTP1 CAR-T cells was significantly reduced, indicating that high expression of hGSTP1 could effectively inhibit generation of cellular reactive oxygen species.

### Example 28. High Expression of hGSTP1 Efficiently Enhancing Anti-Tumor Function of B7H3-CAR-T Cell

### 1. Method

1) Day 0: the cells were inoculated in a 12-well plate. 50000 A549-PCDH were plated in each well. When the tumor cells adhered to the wall (about 5 h), different amounts of T (T cells were added according to the positive rate) were added according to effector-to-target ratios of 1:1, 1:2.5, and 1:5. The culture medium was L500 complete culture medium (without cytokines). When the tumor cells were plated, 1 mL of the culture medium was firstly added. After the T cells were added, the volume of each well was brought to 3 mL.
2) Day 1-3: cell observing: the cell killing condition was observed under a microscope every day. The cell termination time was determined according to the killing progress. The cells in the well were collected to perform flow cytometry detection on the ratio of the T cells and the tumor cells.
3) Day 3: the cells in each well were gently pipetted, the cell supernatant was transferred to a 15 mL centrifuge tube, washed once with 1 mL PBS, and transferred to the 15 mL centrifuge tube described above; the remaining tumor cells were digested with pancreatin, transferred to the 15 mL centrifuge tube described above, and centrifuged at 1800 rpm for 5 min; the cells were collected, and the supernatant was discarded; 30-50 L of CD3 antibodies diluted with PBS were added, mixed well by shaking, and stained at 4° C. for 15 min; the mixture was added with 1 mL PBS, washed once, and centrifuged at 1800 rpm for 5 min, and the supernatant was discarded; a live/dead cell dye was added to label dead cells (incubated at 4° C. for 30 min), washed with 1 mL PBS once, and centrifuged at 1800 rpm for 5 min, and the supernatant was discarded; the cells were added with 400 L PBS, resuspended, filtered through a 300-mesh screen into a flow cytometry tube, and loaded on the machine for assay; the killing effect was analyzed by flow cytometry.

### 2. Results

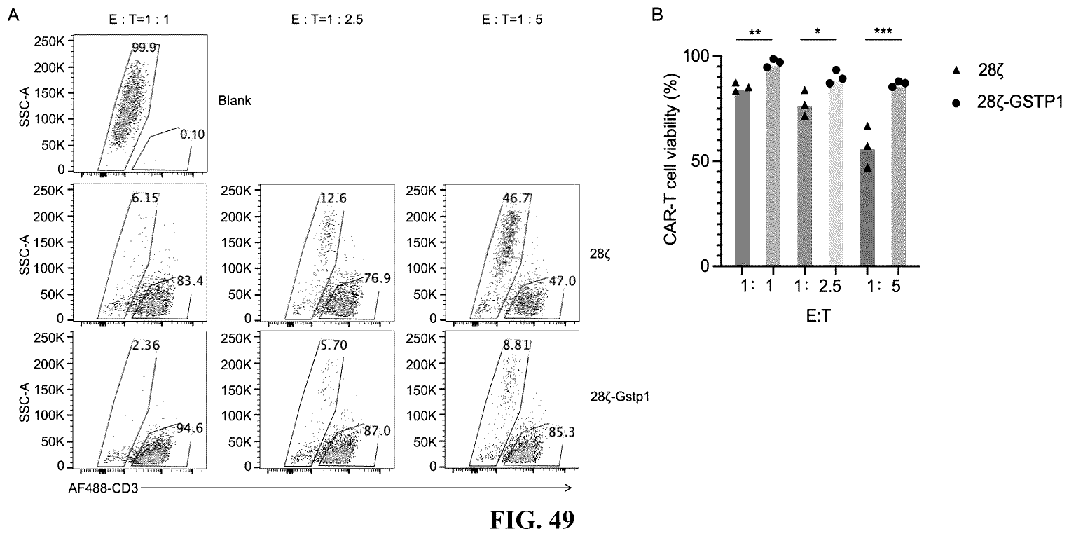

The results of the experiment are shown in FIG. 49. The lung cancer cell A549 cell expressing the B7H3 target was simultaneously added with corresponding amounts of 282-CAR-T and 28ζ-hGSTP1-CAR-T cells according to different effector-to-target ratios of 1:1, 1:2.5, and 1:5. The co-culture killing results show that: the survival rate of the 282-hGSTP1-CAR-T cells was significantly higher than that of the control group. The results indicate that the 28ζ-hGSTP1-CAR-T cells were able to kill tumor cells more strongly. It is shown that the 28ζ-hGSTP1-CAR-T cells can efficiently kill tumor cells while inhibiting the generation of reactive oxygen species, and may have better killing effect in a solid tumor microenvironment.

### Example 29. Validation of In Vivo Inhibition of Lung Cancer Subcutaneously Transplanted Tumor by the B7H3-CAR-T Cell with High Expression of hGTSP1

### 1. Method

1) 4-6 weeks old NCG female mice were injected subcutaneously 150 μL of cell suspension containing 5×10⁶ human lung cancer cell A549 into the right dorsal side of the mice;
2) the growth condition of the subcutaneously transplanted tumor was continuously observed, and when the tumor body gradually enlarged, the long diameter (a) and the short diameter (b) of the tumor body were measured using a vernier caliper. The volume of the tumor body is a×b²/2.
3) when the tumor body size was about 100-200 mm³, the mice were randomly divided into 5 groups;
4) the prepared 28ζ and 28ζ-hGSTP1 CAR-T cells were respectively administered to tumor-bearing mice for tail vein injection treatment according to the dose of 5×10^{6/}100 μL and 1×10⁷/100 μL, and PBS group was used as a control;
5) every 3 to 4 days, the body weight of the mice and the change of the volume of the beared tumor were measured, and the comprehensive condition in the treatment process was observed.

### 2. Results

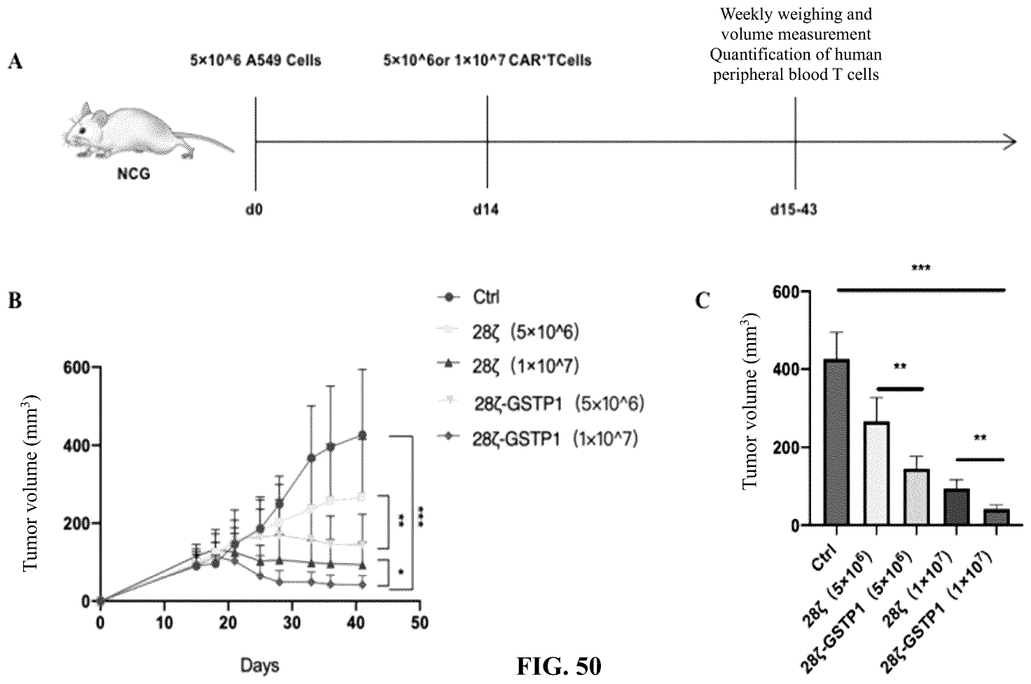

The experimental results are shown in FIG. 50. A lung cancer NCG mouse subcutaneously transplanted tumor model was established. When the volume of the beared tumor of the mice was 100-200 mm³, the mice were randomly divided into 5 groups (PBS, 5×10⁶ 28ζ, 1×10⁷²⁸ζ, 5×10⁶28ζ-hGSTP1, 1×10⁷ 28-hGSTP1) of 6, and administered with a tail vein injection of 5×10⁶ or 1×10⁷ of a therapeutic dose of 28ζ or 28ζ-hGSTP1 CAR-T cells. The PBS group was the control group. The body weight of the mice, the change of the volume of the beared tumor, and the comprehensive condition in the treatment process were continuously detected. From the formation of the subcutaneously transplanted tumor, the body weight of the mice and the size change of the transplanted tumor were measured and recorded every three days. The volume of the transplanted tumor was calculated, and the tumor growth curve was drawn according to the time axis. The results show that: the 28ζ-hGSTP1 CAR-T cells could kill lung cancer transplanted tumor with high efficiency under the condition of a relatively low dose, indicating that the tumor killing effect of the 28ζ-hGSTP1 CAR-T cells was significantly better than that of the 28ζ CAR-T cells.

The preferred embodiments of the present application have been described above in detail, but the present application is not limited to the embodiments. Those skilled in the art can make various equivalent modifications or replacements without violating the spirit of the present application. These equivalent modifications or replacements are included in the scope defined by the claims of the present application.

1. An isolated fully human monoclonal antibody or an antigen-binding fragment thereof, wherein the antibody or the antigen-binding fragment thereof specifically binds to B7H3;
the antibody or the antigen-binding fragment thereof comprises an HCVR and an LCVR;
the HCVR comprises an HCDR1, an HCDR2 and an HCDR3;
the LCVR comprises an LCDR1, an LCDR2 and an LCDR3;
the HCDR1, the HCDR2 and the HCDR3 are an HCDR1, an HCDR2 and an HCDR3, respectively, in an HCVR with an amino acid sequence set forth in SEQ ID NO: 7 or SEQ ID NO: 8; and
the LCDR1, the LCDR2 and the LCDR3 are an LCDR1, an LCDR2 and an LCDR3, respectively, in an LCVR with an amino acid sequence set forth in SEQ ID NO: 17 or SEQ ID NO: 18.

2. The antibody or the antigen-binding fragment thereof according to claim 1, wherein the HCDR1 comprises an amino acid sequence set forth in SEQ ID NO: 1 or SEQ ID NO: 2, or an amino acid sequence having at least 95%, at least 96%, at least 97%, at least 98% or at least 99% identity to SEQ ID NO: 1 or SEQ ID NO: 2;
the HCDR2 comprises an amino acid sequence set forth in SEQ ID NO: 3 or SEQ ID NO: 4, or an amino acid sequence having at least 95%, at least 96%, at least 97%, at least 98% or at least 99% identity to SEQ ID NO: 3 or SEQ ID NO: 4;
the HCDR3 comprises an amino acid sequence set forth in SEQ ID NO: 5 or SEQ ID NO: 6, or an amino acid sequence having at least 95%, at least 96%, at least 97%, at least 98% or at least 99% identity to SEQ ID NO: 5 or SEQ ID NO: 6;
the LCDR1 comprises an amino acid sequence set forth in SEQ ID NO: 11 or SEQ ID NO: 12, or an amino acid sequence having at least 95%, at least 96%, at least 97%, at least 98% or at least 99% identity to SEQ ID NO: 11 or SEQ ID NO: 12;
the LCDR2 comprises an amino acid sequence set forth in SEQ ID NO: 13 or SEQ ID NO: 14, or an amino acid sequence having at least 95%, at least 96%, at least 97%, at least 98% or at least 99% identity to SEQ ID NO: 13 or SEQ ID NO: 14; and
the LCDR3 comprises an amino acid sequence set forth in SEQ ID NO: 15 or SEQ ID NO: 16, or an amino acid sequence having at least 95%, at least 96%, at least 97%, at least 98% or at least 99% identity to SEQ ID NO: 15 or SEQ ID NO: 16.

3. The antibody or the antigen-binding fragment thereof according to claim 2, wherein the HCVR of the antibody or the antigen-binding fragment thereof and the LCVR of the antibody or the antigen-binding fragment thereof are linked by a Linker; and
the Linker has an amino acid sequence set forth in SEQ ID NO: 21 or SEQ ID NO: 22.

4. The antibody or the antigen-binding fragment thereof according to claim 3, wherein the antibody or the antigen-binding fragment thereof has an amino acid sequence set forth in SEQ ID NO: 25 or SEQ ID NO: 26.

5. A fully human chimeric antigen receptor targeting B7H3, comprising the antibody or the antigen-binding fragment thereof according to claim 1.

6. The chimeric antigen receptor according to claim 5, further comprising a transmembrane domain, an intracellular signaling domain, a hinge region, a signal peptide, and/or a co-stimulatory signaling domain.

7. The chimeric antigen receptor according to claim 6, wherein the transmembrane domain comprises transmembrane domains of the following molecules: CD8α, CD28, IgG1, IgG4, 4-1BB, PD-1, CD34, OX40, CD3ζ, IL-2 receptor, IL-7 receptor, and/or IL-11 receptor;
the intracellular signaling domain comprises intracellular signaling domains of the following molecules: CD3ζ, FcRγ, FcRβ, CD3γ, CD3δ, CD3ε, TCRζ, CD4, CD5, CD8, CD21, CD22, CD79a, CD79b, CD278, FcεRI, DAP10, DAP12, CD66d, DAP10, DAP12, and/or FYN;
the hinge region comprises hinge regions of the following molecules: CD8α, CD28, IgG1, IgG4, 4-1BB, PD-1, CD34, OX40, CD3ζ, IL-2 receptor, IL-7 receptor, and/or IL-11 receptor;
the signal peptide comprises signal peptides of the following molecules: a and B chains of a T cell receptor, CD3ζ, CD3ε, CD4, CD5, CD8, CD9, CD28, CD16, CD22, CD33, CD37, CD45, CD64, CD80, CD86, CD134, CD137, CD154, GITR, GM-CSF, ICOS, and/or IgG6; and
the co-stimulatory signaling domain comprises co-stimulatory signaling domains of the following molecules: CD28, ICOS (CD278), CD27, CD19, CD4, CD8α, CD8β, BAFFR, HVEM, LIGHT, KIRDS2, SLAMF7, NKp80 (KLRF1), NKp30, NKp46, CD40, CDS, ICAM-1, 4-1BB (CD137), B7-H3, OX40, DR3, GITR, CD30, TIM1, CD2, CD7, and/or CD226.

8. The chimeric antigen receptor according to claim 7, further comprising a self-cleaving peptide, a TGF-β-antagonizing domain, a safety switch, an immunomodulatory molecule or cytokine, and/or an ROS-inhibiting domain.

9. The chimeric antigen receptor according to claim 8, wherein the self-cleaving peptide comprises T2A, P2A, E2A, and/or F2A;
the TGF-β-antagonizing domain comprises an antibody specifically binding to TGF-β, a nucleic acid molecule encoding a TGF-β signaling-inhibiting protein, and/or human Ski;
the safety switch comprises tEGFR, iCaspase-9, and/or RQR8;
the immunomodulatory molecule or cytokine comprises B7.1, CCL19, CCL21, CD40L, CD137L, GITRL, GM-CSF, IL-12, IL-2, IL-15, IL-18, IL-21, LEC, and/or OX40L; and
the ROS-inhibiting domain comprises a nucleic acid molecule encoding an ROS-inhibiting GSTP1 protein, and/or human GSTP1.

10. The chimeric antigen receptor according to claim 9, wherein the chimeric antigen receptor is selected from any one of the group consisting of:
(1) a chimeric antigen receptor with an amino acid sequence set forth in SEQ ID NO: 56;
(2) a chimeric antigen receptor with an amino acid sequence set forth in SEQ ID NO: 58;
(3) a chimeric antigen receptor with an amino acid sequence set forth in SEQ ID NO: 60;
(4) a chimeric antigen receptor with an amino acid sequence set forth in SEQ ID NO: 62;
(5) a chimeric antigen receptor with an amino acid sequence set forth in SEQ ID NO: 64;
(6) a chimeric antigen receptor with an amino acid sequence set forth in SEQ ID NO: 66;
(7) a chimeric antigen receptor set forth in SEQ ID NO: 68; and
(8) a derived fusion protein formed by a substitution, deletion or addition of one or more amino acids to the amino acid sequence of the chimeric antigen receptor described in (1), (2), (3), (4), (5), (6), or (7).

11. A polynucleotide, having a sequence comprising: a nucleotide sequence encoding the antibody or the antigen-binding fragment thereof according to claim 1, or a complementary sequence thereof.

12. The polynucleotide according to claim 11, wherein the nucleotide sequence encoding the HCVR of the antibody or the antigen-binding fragment thereof is set forth in SEQ ID NO: 9 or SEQ ID NO: 10;
the nucleotide sequence encoding the LCVR of the antibody or the antigen-binding fragment thereof is set forth in SEQ ID NO: 19 or SEQ ID NO: 20.

13. A recombinant vector, comprising the polynucleotide according to claim 11.

14. An engineered host cell, comprising the recombinant vector according to claim 13.

15. The engineered host cell according to claim 14, wherein the immune cell comprises a T cell, a B cell, an NK cell, an iNKT cell, a CTL cell, a dendritic cell, a myeloid cell, a monocyte and a macrophage, or any combination thereof.

16. A derivative, comprising the antibody or the antigen-binding fragment thereof according to claim 1 with a detectable label, the antibody or the antigen-binding fragment thereof according to claim 1 conferring antibiotic resistance, or the antibody or the antigen-binding fragment thereof according to claim 1 bound or coupled to a therapeutic agent.

17. A pharmaceutical composition or biological agent, comprising the engineered host cell according to claim 14.

18. A method for detecting B7H3 in a test sample, comprising the following steps: contacting the test sample with the antibody or the antigen-binding fragment thereof according to claim 1, and detecting formation of a complex by the antibody or the antigen-binding fragment thereof and B7H3.

19. A method for treating a disease or disorder associated with B7H3 in a subject in need thereof, comprising administering a therapeutically effective amount of the engineered host cell according to claim 14 to the subject with the disease or disorder associated with B7H3.

20. The method according to claim 19, wherein the disease or disorder associated with B7H3 comprises a tumor expressing B7H3; and
the tumor comprises ovarian cancer, kidney cancer, lung cancer, breast cancer, colorectal cancer, esophageal cancer, prostate cancer, oral cancer, gastric cancer, pancreatic cancer, endometrial cancer, liver cancer, bladder cancer, osteosarcoma, glioma, acute myeloid leukemia, non-Hodgkin lymphoma, Hodgkin lymphoma, brain cancer, cervical cancer, head and neck cancer, testicular cancer, pituitary cancer, esophagus cancer, skin cancer, bone cancer, B-cell lymphoma, T-cell lymphoma, myeloma, hematopoietic tumor, thymoma, anal cancer, primary or metastatic melanoma, squamous cell cancer, basal cell carcinoma, angiosarcoma, hemangioendothelioma, thyroid cancer, soft tissue sarcoma, gastrointestinal cancer, intrahepatic cholangiocarcinoma, joint cancer, nasal cancer, and/or any other cancer now known or later discovered.


SEQUENCE LISTING (68 sequences)

SEQ ID NO: 1 - protein, 8 aa, synthetic construct
  REGION 1..8: note = synthesized
GFTFSSYA

SEQ ID NO: 2 - protein, 8 aa, synthetic construct
  REGION 1..8: note = synthesized
GFGFSGYA

SEQ ID NO: 3 - protein, 8 aa, synthetic construct
  REGION 1..8: note = synthesized
ISGSGGST

SEQ ID NO: 4 - protein, 8 aa, synthetic construct
  REGION 1..8: note = synthesized
ISGSGGST

SEQ ID NO: 5 - protein, 10 aa, synthetic construct
  REGION 1..10: note = synthesized
ARGVGRGFDY

SEQ ID NO: 6 - protein, 9 aa, synthetic construct
  REGION 1..9: note = synthesized
AKALGAPVY

SEQ ID NO: 7 - protein, 117 aa, synthetic construct
  REGION 1..117: note = synthesized
EVQLFQSGGGLVQPGGSLRLSCAASGFTFSSYAMSWVRQAPGKGLEWVSAISGSGGSTYYADSVKGRFTISRDNSKNTLYLQMNSLRAEDTAVYYCARGVGRGFDYWGQGTTVTVSS

SEQ ID NO: 8 - protein, 115 aa, synthetic construct
  REGION 1..115: note = synthesized
VQLVESGGGLVQPGGSLRLSCAASGFGFSGYAMSWVRQAPGKGLEWVSAISGSGGSTYYADSVKGRFTISRDNSKNTLYLQMNSLRAEDTAVYYCAKALGAPVYWGQGTMVTVSS

SEQ ID NO: 9 - DNA, 351 bp, synthetic construct
  misc_feature 1..351: note = synthesized
  source 1..351: mol_type = other DNA
gaggtgcagctgttccagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgccaggggtgttggccggggctttgactactggggccaggggaccacggtcaccgtctcctca

SEQ ID NO: 10 - DNA, 345 bp, synthetic construct
  misc_feature 1..345: note = synthesized
  source 1..345: mol_type = other DNA
gtgcagctggtggagtctgggggaggcttggtacagcctggggggtccctgagactctcctgtgcagcctctggattcggttttagtggctatgccatgagctgggtccgccaggctccagggaaggggctggagtgggtctcagctatcagtggtagtggtggtagcacatactacgcagactccgtgaagggccggttcaccatctccagagacaattccaagaacacgctgtatctgcaaatgaacagcctgagagccgaggacacggccgtatattactgtgcgaaagctctgggagcaccagtctactggggccagggaacaatggtcaccgtctcctca

SEQ ID NO: 11 - protein, 6 aa, synthetic construct
  REGION 1..6: note = synthesized
QSISIY

SEQ ID NO: 12 - protein, 6 aa, synthetic construct
  REGION 1..6: note = synthesized
QSISSY

SEQ ID NO: 13-14 - skipped (2 entries)

SEQ ID NO: 15 - protein, 10 aa, synthetic construct
  REGION 1..10: note = synthesized
QQTYSTPPWT

SEQ ID NO: 16 - protein, 10 aa, synthetic construct
  REGION 1..10: note = synthesized
QQSYSTPPWT

SEQ ID NO: 17 - protein, 108 aa, synthetic construct
  REGION 1..108: note = synthesized
DIQLTQSPSSLSASVGDRVTITCRASQSISIYLNWYRQQPGKAPKLLIYAASSLQSGVPSRFSGSGSGTDFTLTISSLQPEDFATYFCQQTYSTPPWTFGQGTKVDIK

SEQ ID NO: 18 - protein, 108 aa, synthetic construct
  REGION 1..108: note = synthesized
DIVMTQSPSSLSASVGDRVTITCRASQSISSYLNWYQQKPGKAPKLLIYAASSLQSGVPSRFSGSGSGTDFTLTISSLQPEDFATYYCQQSYSTPPWTFGQGTKVDIK

SEQ ID NO: 19 - DNA, 324 bp, synthetic construct
  misc_feature 1..324: note = synthesized
  source 1..324: mol_type = other DNA
gacatccagttgacccagtctccatcctccctgtctgcatctgtaggagacagagtcaccatcacttgccgggcaagtcagagcattagcatctatttaaattggtatcggcagcaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttacttctgtcaacagacttacagtacccctccgtggacgttcggccaagggaccaaagtggatatcaaa

SEQ ID NO: 20 - DNA, 324 bp, synthetic construct
  misc_feature 1..324: note = synthesized
  source 1..324: mol_type = other DNA
gacatcgtgatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgtggacgttcggccaagggaccaaagtggatatcaaa

SEQ ID NO: 21 - protein, 15 aa, synthetic construct
  REGION 1..15: note = synthesized
SGGGSGGGGSGGGGS

SEQ ID NO: 22 - protein, 15 aa, synthetic construct
  REGION 1..15: note = synthesized
GGGGSGGGGSGGGGS

SEQ ID NO: 23 - DNA, 45 bp, synthetic construct
  misc_feature 1..45: note = synthesized
  source 1..45: mol_type = other DNA
agtggcggtggctctggcggtggtgggtcgggtggcggcggatca

SEQ ID NO: 24 - DNA, 45 bp, synthetic construct
  misc_feature 1..45: note = synthesized
  source 1..45: mol_type = other DNA
ggtggcggtggctcgggcggtggtgggtcgggtggcggcggatca

SEQ ID NO: 25 - protein, 240 aa, synthetic construct
  REGION 1..240: note = synthesized
EVQLFQSGGGLVQPGGSLRLSCAASGFTFSSYAMSWVRQAPGKGLEWVSAISGSGGSTYYADSVKGRFTISRDNSKNTLYLQMNSLRAEDTAVYYCARGVGRGFDYWGQGTTVTVSSSGGGSGGGGSGGGGSDIQLTQSPSSLSASVGDRVTITCRASQSISIYLNWYRQQPGKAPKLLIYAASSLQSGVPSRFSGSGSGTDFTLTISSLQPEDFATYFCQQTYSTPPWTFGQGTKVDIK

SEQ ID NO: 26 - protein, 238 aa, synthetic construct
  REGION 1..238: note = synthesized
VQLVESGGGLVQPGGSLRLSCAASGFGFSGYAMSWVRQAPGKGLEWVSAISGSGGSTYYADSVKGRFTISRDNSKNTLYLQMNSLRAEDTAVYYCAKALGAPVYWGQGTMVTVSSGGGGSGGGGSGGGGSDIVMTQSPSSLSASVGDRVTITCRASQSISSYLNWYQQKPGKAPKLLIYAASSLQSGVPSRFSGSGSGTDFTLTISSLQPEDFATYYCQQSYSTPPWTFGQGTKVDIK

SEQ ID NO: 27 - DNA, 720 bp, synthetic construct
  misc_feature 1..720: note = synthesized
  source 1..720: mol_type = other DNA
gaggtgcagctgttccagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgccaggggtgttggccggggctttgactactggggccaggggaccacggtcaccgtctcctcaagtggcggtggctctggcggtggtgggtcgggtggcggcggatcagacatccagttgacccagtctccatcctccctgtctgcatctgtaggagacagagtcaccatcacttgccgggcaagtcagagcattagcatctatttaaattggtatcggcagcaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttacttctgtcaacagacttacagtacccctccgtggacgttcggccaagggaccaaagtggatatcaaa

SEQ ID NO: 28 - DNA, 714 bp, synthetic construct
  misc_feature 1..714: note = synthesized
  source 1..714: mol_type = other DNA
gtgcagctggtggagtctgggggaggcttggtacagcctggggggtccctgagactctcctgtgcagcctctggattcggttttagtggctatgccatgagctgggtccgccaggctccagggaaggggctggagtgggtctcagctatcagtggtagtggtggtagcacatactacgcagactccgtgaagggccggttcaccatctccagagacaattccaagaacacgctgtatctgcaaatgaacagcctgagagccgaggacacggccgtatattactgtgcgaaagctctgggagcaccagtctactggggccagggaacaatggtcaccgtctcctcaggtggcggtggctcgggcggtggtgggtcgggtggcggcggatcagacatcgtgatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgtggacgttcggccaagggaccaaagtggatatcaaa

SEQ ID NO: 29 - protein, 22 aa, synthetic construct
  REGION 1..22: note = synthesized
IWAPLAGTCGVLLLSLVITLYC

SEQ ID NO: 30 - DNA, 66 bp, synthetic construct
  misc_feature 1..66: note = synthesized
  source 1..66: mol_type = other DNA
atctgggcgcccttggccgggacttgtggggtccttctcctgtcactggttatcaccctttactgc

SEQ ID NO: 31 - protein, 112 aa, synthetic construct
  REGION 1..112: note = synthesized
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - DNA, 336 bp, synthetic construct
  misc_feature 1..336: note = synthesized
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 33 - protein, 47 aa, synthetic construct
  REGION 1..47: note = synthesized
TTTPAPRPPTPAPTIASQPLSLRPEACRPAAGGAVHTRGLDFACDIY

SEQ ID NO: 34 - DNA, 141 bp, synthetic construct
  misc_feature 1..141: note = synthesized
  source 1..141: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctac

SEQ ID NO: 35 - protein, 19 aa, synthetic construct
  REGION 1..19: note = synthesized
MEFGLSWLFLVAILKGVQC

SEQ ID NO: 36 - DNA, 57 bp, synthetic construct
  misc_feature 1..57: note = synthesized
  source 1..57: mol_type = other DNA
atggaatttggcctgagctggctgtttctggtggcgattctgaaaggcgtgcagtgc

SEQ ID NO: 37 - protein, 41 aa, synthetic construct
  REGION 1..41: note = synthesized
RSKRSRLLHSDYMNMTPRRPGPTRKHYQPYAPPRDFAAYRS

SEQ ID NO: 38 - DNA, 123 bp, synthetic construct
  misc_feature 1..123: note = synthesized
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 39 - protein, 48 aa, synthetic construct
  REGION 1..48: note = synthesized
RFSVVKRGRKKLLYIFKQPFMRPVQTTQEEDGCSCRFPEEEEGGCELR

SEQ ID NO: 40 - DNA, 144 bp, synthetic construct
  misc_feature 1..144: note = synthesized
  source 1..144: mol_type = other DNA
aggttcagtgtcgtgaagagaggccggaagaagctgctgtacatcttcaagcagcctttcatgaggcccgtgcagactacccaggaggaagatggatgcagctgtagattccctgaagaggaggaaggaggctgtgagctgaga

SEQ ID NO: 41 - protein, 21 aa, synthetic construct
  REGION 1..21: note = synthesized
GSGEGRGSLLTCGDVEENPGP

SEQ ID NO: 42 - DNA, 63 bp, synthetic construct
  misc_feature 1..63: note = synthesized
  source 1..63: mol_type = other DNA
ggatctggagagggcagaggaagtcttctaacatgcggtgacgtggaggagaatcccggccct

SEQ ID NO: 43 - protein, 728 aa, synthetic construct
  REGION 1..728: note = synthesized
MEAAAGGRGCFQPHPGLQKTLEQFHLSSMSSLGGPAAFSARWAQEAYKKESAKEAGAAAVPAPVPAATEPPPVLHLPAIQPPPPVLPGPFFMPSDRSTERCETVLEGETISCFVVGGEKRLCLPQILNSVLRDFSLQQINAVCDELHIYCSRCTADQLEILKVMGILPFSAPSCGLITKTDAERLCNALLYGGAYPPPCKKELAASLALGLELSERSVRVYHECFGKCKGLLVPELYSSPSAACIQCLDCRLMYPPHKFVVHSHKALENRTCHWGFDSANWRAYILLSQDYTGKEEQARLGRCLDDVKEKFDYGNKYKRRVPRVSSEPPASIRPKTDDTSSQSPAPSEKDKPSSWLRTLAGSSNKSLGCVHPRQRLSAFRPWSPAVSASEKELSPHLPALIRDSFYSYKSFETAVAPNVALAPPAQQKVVSSPPCAAAVSRAPEPLATCTQPRKRKLTVDTPGAPETLAPVAAPEEDKDSEAEVEVESREEFTSSLSSLSSPSFTSSSSAKDLGSPGARALPSAVPDAAAPADAPSGLEAELEHLRQALEGGLDTKEAKEKFLHEVVKMRVKQEEKLSAALQAKRSLHQELEFLRVAKKEKLREATEAKRNLRKEIERLRAENEKKMKEANESRLRLKRELEQARQARVCDKGCEAGRLRAKYSAQIEDLQVKLQHAEADREQLRADLLREREAREHLEKVVKELQEQLWPRARPEAAGSEGAAELEP

SEQ ID NO: 44 - DNA, 2187 bp, synthetic construct
  misc_feature 1..2187: note = synthesized
  source 1..2187: mol_type = other DNA
atggaggcggcggcaggcggccgcggctgtttccagccgcacccggggctgcagaagacgctggagcagttccacctgagctccatgagctcgctgggcggcccggccgctttctcggcgcgctgggcgcaggaggcctacaagaaggagagcgccaaggaggcgggcgcggccgcggtgccggcgccggtgcccgcagccaccgagccgccgcccgtgctgcacctgcccgccatccagccgccgccgcccgtgctgcccgggcccttcttcatgccgtccgaccgctccaccgagcgctgcgagaccgtactggaaggcgagaccatctcgtgcttcgtggtgggaggcgagaagcgcctgtgtctgccgcagattctcaactcggtgctgcgcgacttctcgctgcagcagatcaacgcggtgtgcgacgagctccacatctactgctcgcgctgcacggccgaccagctggagatcctcaaagtcatgggcatcctgcccttctcggcgccctcgtgcgggctcatcaccaagacggacgccgagcgcctgtgcaacgcgctgctctacggcggcgcctacccgccgccctgcaagaaggagctggccgccagcctggcgctgggcctggagctcagcgagcgcagcgtccgcgtgtaccacgagtgcttcggcaagtgtaaggggctgctggtgcccgagctctacagcagcccgagcgccgcctgcatccagtgcctggactgccgcctcatgtacccgccgcacaagttcgtggtgcactcgcacaaggccctggagaaccggacctgccactggggcttcgactcggccaactggcgggcctacatcctgctgagccaggattacacgggcaaggaggagcaggcgcgcctcggccgctgcctggacgacgtgaaggagaaattcgactatggcaacaagtacaagcggcgggtgccccgggtctcctctgagcctccggcctccataagacccaaaacagatgacacctcttcccagtcccccgcgccttccgaaaaggacaagccgtccagctggctgcggaccttggccggctcttccaataagagcctgggctgtgttcaccctcgccagcgcctctctgctttccgaccctggtcccccgcagtgtcagcgagtgagaaagagctctccccacacctcccggccctcatccgagacagcttctactcctacaagagctttgagacagccgtggcgcccaacgtggccctcgcaccgccggcccagcagaaggttgtgagcagccctccgtgtgccgccgccgtctcccgggcccccgagcctctcgccacttgcacccagcctcggaagcggaagctgactgtggacaccccaggagccccagagacgctggcgcccgtggctgccccagaggaggacaaggactcggaggcggaggtggaagttgaaagcagggaggaattcacctcctccttgtcctcgctctcttccccgtcctttacctcatccagctccgccaaggacctgggctccccgggtgcgcgtgccctgccctcggccgtccctgatgctgcggcccctgccgacgcccccagtgggctggaggcggagctggagcacctgcggcaggcactggagggcggcctggacaccaaggaagccaaagagaagttcctgcatgaggtggtcaagatgcgcgtgaagcaggaggagaagctcagcgcagccctgcaggccaagcgcagcctccaccaggagctggagttcctacgcgtggccaagaaggagaagctgcgggaggccacggaggccaagcgtaacctgcggaaggagatcgagcgtctccgcgccgagaacgagaagaagatgaaagaggccaacgagtcacggctgcgcctgaagcgggagctggagcaggcgcggcaggcccgggtgtgcgacaagggctgcgaggcgggccgcctgcgcgccaagtactcggcccagatcgaagacctgcaggtgaagctgcagcacgcggaggcggaccgggagcagctgcgggccgacctgctgcgggagcgcgaggcccgggagcacctggagaaggtggtgaaggagctgcaggaacagctgtggccgcgggcccgccccgaggctgcgggcagcgagggcgctgcggagctggagccgtag

SEQ ID NO: 45 - protein, 352 aa, synthetic construct
  REGION 1..352: note = synthesized
MWLQSLLLLGTVACSISRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 46 - DNA, 1059 bp, synthetic construct
  misc_feature 1..1059: note = synthesized
  source 1..1059: mol_type = other DNA
atgtggctgcagagcctgctgctcttgggcactgtggcctgcagcatctctcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtaa

SEQ ID NO: 47 - protein, 162 aa, synthetic construct
  REGION 1..162: note = synthesized
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 48 - DNA, 489 bp, synthetic construct
  misc_feature 1..489: note = synthesized
  source 1..489: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagaccttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 49 - protein, 162 aa, synthetic construct
  REGION 1..162: note = synthesized
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 50 - DNA, 489 bp, synthetic construct
  misc_feature 1..489: note = synthesized
  source 1..489: mol_type = other DNA
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctga

SEQ ID NO: 51 - protein, 210 aa, synthetic construct
  REGION 1..210: note = synthesized
MPPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYISLIYTNYEAGKDDYVKALPGQLKPFETLLSQNQGGKTFIVGDQISFADYNLLDLLLIHEVLAPGCLDAFPLLSAYVGRLSARPKLKAFLASPEYVNLPINGNGKQ

SEQ ID NO: 52 - DNA, 633 bp, synthetic construct
  misc_feature 1..633: note = synthesized
  source 1..633: mol_type = other DNA
atgccgccctacaccgtggtctatttcccagttcgaggccgctgcgcggccctgcgcatgctgctggcagatcagggccagagctggaaggaggaggtggtgaccgtggagacgtggcaggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactatgaggcgggcaaggatgactatgtgaaggcactgcccgggcaactgaagccttttgagaccctgctgtcccagaaccagggaggcaagaccttcattgtgggagaccagatctccttcgctgactacaacctgctggacttgctgctgatccatgaggtcctagcccctggctgcctggatgcgttccccctgctctcagcatatgtggggcgcctcagtgcccggcccaagctcaaggccttcctggcctcccctgagtacgtgaacctccccatcaatggcaacgggaaacagtga

SEQ ID NO: 53 - protein, 1234 aa, synthetic construct
  REGION 1..1234: note = synthesized
MEFGLSWLFLVAILKGVQCEVQLFQSGGGLVQPGGSLRLSCAASGFTFSSYAMSWVRQAPGKGLEWVSAISGSGGSTYYADSVKGRFTISRDNSKNTLYLQMNSLRAEDTAVYYCARGVGRGFDYWGQGTTVTVSSSGGGSGGGGSGGGGSDIQLTQSPSSLSASVGDRVTITCRASQSISIYLNWYRQQPGKAPKLLIYAASSLQSGVPSRFSGSGSGTDFTLTISSLQPEDFATYFCQQTYSTPPWTFGQGTKVDIKTRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRACGSGEGRGSLLTCGDVEENPGPMEAAAGGRGCFQPHPGLQKTLEQFHLSSMSSLGGPAAFSARWAQEAYKKESAKEAGAAAVPAPVPAATEPPPVLHLPAIQPPPPVLPGPFFMPSDRSTERCETVLEGETISCFVVGGEKRLCLPQILNSVLRDFSLQQINAVCDELHIYCSRCTADQLEILKVMGILPFSAPSCGLITKTDAERLCNALLYGGAYPPPCKKELAASLALGLELSERSVRVYHECFGKCKGLLVPELYSSPSAACIQCLDCRLMYPPHKFVVHSHKALENRTCHWGFDSANWRAYILLSQDYTGKEEQARLGRCLDDVKEKFDYGNKYKRRVPRVSSEPPASIRPKTDDTSSQSPAPSEKDKPSSWLRTLAGSSNKSLGCVHPRQRLSAFRPWSPAVSASEKELSPHLPALIRDSFYSYKSFETAVAPNVALAPPAQQKVVSSPPCAAAVSRAPEPLATCTQPRKRKLTVDTPGAPETLAPVAAPEEDKDSEAEVEVESREEFTSSLSSLSSPSFTSSSSAKDLGSPGARALPSAVPDAAAPADAPSGLEAELEHLRQALEGGLDTKEAKEKFLHEVVKMRVKQEEKLSAALQAKRSLHQELEFLRVAKKEKLREATEAKRNLRKEIERLRAENEKKMKEANESRLRLKRELEQARQARVCDKGCEAGRLRAKYSAQIEDLQVKLQHAEADREQLRADLLREREAREHLEKVVKELQEQLWPRARPEAAGSEGAAELEP

SEQ ID NO: 54 - DNA, 3705 bp, synthetic construct
  misc_feature 1..3705: note = synthesized
  source 1..3705: mol_type = other DNA
atggaatttggcctgagctggctgtttctggtggcgattctgaaaggcgtgcagtgcgaggtgcagctgttccagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgccaggggtgttggccggggctttgactactggggccaggggaccacggtcaccgtctcctcaagtggcggtggctctggcggtggtgggtcgggtggcggcggatcagacatccagttgacccagtctccatcctccctgtctgcatctgtaggagacagagtcaccatcacttgccgggcaagtcagagcattagcatctatttaaattggtatcggcagcaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttacttctgtcaacagacttacagtacccctccgtggacgttcggccaagggaccaaagtggatatcaaaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcatgcggatctggagagggcagaggaagtcttctaacatgcggtgacgtggaggagaatcccggccctatggaggcggcggcaggcggccgcggctgtttccagccgcacccggggctgcagaagacgctggagcagttccacctgagctccatgagctcgctgggcggcccggccgctttctcggcgcgctgggcgcaggaggcctacaagaaggagagcgccaaggaggcgggcgcggccgcggtgccggcgccggtgcccgcagccaccgagccgccgcccgtgctgcacctgcccgccatccagccgccgccgcccgtgctgcccgggcccttcttcatgccgtccgaccgctccaccgagcgctgcgagaccgtactggaaggcgagaccatctcgtgcttcgtggtgggaggcgagaagcgcctgtgtctgccgcagattctcaactcggtgctgcgcgacttctcgctgcagcagatcaacgcggtgtgcgacgagctccacatctactgctcgcgctgcacggccgaccagctggagatcctcaaagtcatgggcatcctgcccttctcggcgccctcgtgcgggctcatcaccaagacggacgccgagcgcctgtgcaacgcgctgctctacggcggcgcctacccgccgccctgcaagaaggagctggccgccagcctggcgctgggcctggagctcagcgagcgcagcgtccgcgtgtaccacgagtgcttcggcaagtgtaaggggctgctggtgcccgagctctacagcagcccgagcgccgcctgcatccagtgcctggactgccgcctcatgtacccgccgcacaagttcgtggtgcactcgcacaaggccctggagaaccggacctgccactggggcttcgactcggccaactggcgggcctacatcctgctgagccaggattacacgggcaaggaggagcaggcgcgcctcggccgctgcctggacgacgtgaaggagaaattcgactatggcaacaagtacaagcggcgggtgccccgggtctcctctgagcctccggcctccataagacccaaaacagatgacacctcttcccagtcccccgcgccttccgaaaaggacaagccgtccagctggctgcggaccttggccggctcttccaataagagcctgggctgtgttcaccctcgccagcgcctctctgctttccgaccctggtcccccgcagtgtcagcgagtgagaaagagctctccccacacctcccggccctcatccgagacagcttctactcctacaagagctttgagacagccgtggcgcccaacgtggccctcgcaccgccggcccagcagaaggttgtgagcagccctccgtgtgccgccgccgtctcccgggcccccgagcctctcgccacttgcacccagcctcggaagcggaagctgactgtggacaccccaggagccccagagacgctggcgcccgtggctgccccagaggaggacaaggactcggaggcggaggtggaagttgaaagcagggaggaattcacctcctccttgtcctcgctctcttccccgtcctttacctcatccagctccgccaaggacctgggctccccgggtgcgcgtgccctgccctcggccgtccctgatgctgcggcccctgccgacgcccccagtgggctggaggcggagctggagcacctgcggcaggcactggagggcggcctggacaccaaggaagccaaagagaagttcctgcatgaggtggtcaagatgcgcgtgaagcaggaggagaagctcagcgcagccctgcaggccaagcgcagcctccaccaggagctggagttcctacgcgtggccaagaaggagaagctgcgggaggccacggaggccaagcgtaacctgcggaaggagatcgagcgtctccgcgccgagaacgagaagaagatgaaagaggccaacgagtcacggctgcgcctgaagcgggagctggagcaggcgcggcaggcccgggtgtgcgacaagggctgcgaggcgggccgcctgcgcgccaagtactcggcccagatcgaagacctgcaggtgaagctgcagcacgcggaggcggaccgggagcagctgcgggccgacctgctgcgggagcgcgaggcccgggagcacctggagaaggtggtgaaggagctgcaggaacagctgtggccgcgggcccgccccgaggctgcgggcagcgagggcgctgcggagctggagccgtag

SEQ ID NO: 55 - protein, 670 aa, synthetic construct
  REGION 1..670: note = synthesized
MEFGLSWLFLVAILKGVQCEVQLFQSGGGLVQPGGSLRLSCAASGFTFSSYAMSWVRQAPGKGLEWVSAISGSGGSTYYADSVKGRFTISRDNSKNTLYLQMNSLRAEDTAVYYCARGVGRGFDYWGQGTTVTVSSSGGGSGGGGSGGGGSDIQLTQSPSSLSASVGDRVTITCRASQSISIYLNWYRQQPGKAPKLLIYAASSLQSGVPSRFSGSGSGTDFTLTISSLQPEDFATYFCQQTYSTPPWTFGQGTKVDIKTRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRACGSGEGRGSLLTCGDVEENPGPRTMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 56 - DNA, 2013 bp, synthetic construct
  misc_feature 1..2013: note = synthesized
  source 1..2013: mol_type = other DNA
atggaatttggcctgagctggctgtttctggtggcgattctgaaaggcgtgcagtgcgaggtgcagctgttccagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgccaggggtgttggccggggctttgactactggggccaggggaccacggtcaccgtctcctcaagtggcggtggctctggcggtggtgggtcgggtggcggcggatcagacatccagttgacccagtctccatcctccctgtctgcatctgtaggagacagagtcaccatcacttgccgggcaagtcagagcattagcatctatttaaattggtatcggcagcaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttacttctgtcaacagacttacagtacccctccgtggacgttcggccaagggaccaaagtggatatcaaaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcatgcggatctggagagggcagaggaagtcttctaacatgcggtgacgtggaggagaatcccggccctcgtacgatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagaccttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 57 - protein, 670 aa, synthetic construct
  REGION 1..670: note = synthesized
MEFGLSWLFLVAILKGVQCEVQLFQSGGGLVQPGGSLRLSCAASGFTFSSYAMSWVRQAPGKGLEWVSAISGSGGSTYYADSVKGRFTISRDNSKNTLYLQMNSLRAEDTAVYYCARGVGRGFDYWGQGTTVTVSSSGGGSGGGGSGGGGSDIQLTQSPSSLSASVGDRVTITCRASQSISIYLNWYRQQPGKAPKLLIYAASSLQSGVPSRFSGSGSGTDFTLTISSLQPEDFATYFCQQTYSTPPWTFGQGTKVDIKTRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRACGSGEGRGSLLTCGDVEENPGPRTMRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 58 - DNA, 2013 bp, synthetic construct
  misc_feature 1..2013: note = synthesized
  source 1..2013: mol_type = other DNA
atggaatttggcctgagctggctgtttctggtggcgattctgaaaggcgtgcagtgcgaggtgcagctgttccagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgccaggggtgttggccggggctttgactactggggccaggggaccacggtcaccgtctcctcaagtggcggtggctctggcggtggtgggtcgggtggcggcggatcagacatccagttgacccagtctccatcctccctgtctgcatctgtaggagacagagtcaccatcacttgccgggcaagtcagagcattagcatctatttaaattggtatcggcagcaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttacttctgtcaacagacttacagtacccctccgtggacgttcggccaagggaccaaagtggatatcaaaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcatgcggatctggagagggcagaggaagtcttctaacatgcggtgacgtggaggagaatcccggccctcgtacgatgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctga

SEQ ID NO: 59 - protein, 670 aa, synthetic construct; identical to SEQ ID NO: 55
  REGION 1..670: note = synthesized

SEQ ID NO: 60 - DNA, 2013 bp, synthetic construct; identical to SEQ ID NO: 56
  misc_feature 1..2013: note = synthesized
  source 1..2013: mol_type = other DNA

SEQ ID NO: 61 - protein, 863 aa, synthetic construct
  REGION 1..863: note = synthesized
MEFGLSWLFLVAILKGVQCVQLVESGGGLVQPGGSLRLSCAASGFGFSGYAMSWVRQAPGKGLEWVSAISGSGGSTYYADSVKGRFTISRDNSKNTLYLQMNSLRAEDTAVYYCAKALGAPVYWGQGTMVTVSSGGGGSGGGGSGGGGSDIVMTQSPSSLSASVGDRVTITCRASQSISSYLNWYQQKPGKAPKLLIYAASSLQSGVPSRFSGSGSGTDFTLTISSLQPEDFATYYCQQSYSTPPWTFGQGTKVDIKTRTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRACGSGEGRGSLLTCGDVEENPGPMWLQSLLLLGTVACSISRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 62 - DNA, 2592 bp, synthetic construct
  misc_feature 1..2592: note = synthesized
  source 1..2592: mol_type = other DNA
atggaatttggcctgagctggctgtttctggtggcgattctgaaaggcgtgcagtgcgtgcagctggtggagtctgggggaggcttggtacagcctggggggtccctgagactctcctgtgcagcctctggattcggttttagtggctatgccatgagctgggtccgccaggctccagggaaggggctggagtgggtctcagctatcagtggtagtggtggtagcacatactacgcagactccgtgaagggccggttcaccatctccagagacaattccaagaacacgctgtatctgcaaatgaacagcctgagagccgaggacacggccgtatattactgtgcgaaagctctgggagcaccagtctactggggccagggaacaatggtcaccgtctcctcaggtggcggtggctcgggcggtggtgggtcgggtggcggcggatcagacatcgtgatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccgtggacgttcggccaagggaccaaagtggatatcaaaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggttcagtgtcgtgaagagaggccggaagaagctgctgtacatcttcaagcagcctttcatgaggcccgtgcagactacccaggaggaagatggatgcagctgtagattccctgaagaggaggaaggaggctgtgagctgagaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcatgcggatctggagagggcagaggaagtcttctaacatgcggtgacgtggaggagaatcccggccctatgtggctgcagagcctgctgctcttgggcactgtggcctgcagcatctctcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtaa

SEQ ID NO: 63 - protein, 865 aa, synthetic construct
  REGION 1..865: note = synthesized
MEFGLSWLFLVAILKGVQCEVQLFQSGGGLVQPGGSLRLSCAASGFTFSSYAMSWVRQAPGKGLEWVSAISGSGGSTYYADSVKGRFTISRDNSKNTLYLQMNSLRAEDTAVYYCARGVGRGFDYWGQGTTVTVSSSGGGSGGGGSGGGGSDIQLTQSPSSLSASVGDRVTITCRASQSISIYLNWYRQQPGKAPKLLIYAASSLQSGVPSRFSGSGSGTDFTLTISSLQPEDFATYFCQQTYSTPPWTFGQGTKVDIKTRTTTPAPRPPTPAPTIASQPLSLRPEACRPAAGGAVHTRGLDFACDIYIWAPLAGTCGVLLLSLVITLYCRFSVVKRGRKKLLYIFKQPFMRPVQTTQEEDGCSCRFPEEEEGGCELRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRACGSGEGRGSLLTCGDVEENPGPMWLQSLLLLGTVACSISRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 64 - DNA, 2598 bp, synthetic construct
  misc_feature 1..2598: note = synthesized
  source 1..2598: mol_type = other DNA
atggaatttggcctgagctggctgtttctggtggcgattctgaaaggcgtgcagtgcgaggtgcagctgttccagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgccaggggtgttggccggggctttgactactggggccaggggaccacggtcaccgtctcctcaagtggcggtggctctggcggtggtgggtcgggtggcggcggatcagacatccagttgacccagtctccatcctccctgtctgcatctgtaggagacagagtcaccatcacttgccgggcaagtcagagcattagcatctatttaaattggtatcggcagcaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttacttctgtcaacagacttacagtacccctccgtggacgttcggccaagggaccaaagtggatatcaaaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggttcagtgtcgtgaagagaggccggaagaagctgctgtacatcttcaagcagcctttcatgaggcccgtgcagactacccaggaggaagatggatgcagctgtagattccctgaagaggaggaaggaggctgtgagctgagaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcatgcggatctggagagggcagaggaagtcttctaacatgcggtgacgtggaggagaatcccggccctatgtggctgcagagcctgctgctcttgggcactgtggcctgcagcatctctcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtaa

SEQ ID NO: 65 - protein, 716 aa, synthetic construct
  REGION 1..716: note = synthesized
MEFGLSWLFLVAILKGVQCEVQLFQSGGGLVQPGGSLRLSCAASGFTFSSYAMSWVRQAPGKGLEWVSAISGSGGSTYYADSVKGRFTISRDNSKNTLYLQMNSLRAEDTAVYYCARGVGRGFDYWGQGTTVTVSSSGGGSGGGGSGGGGSDIQLTQSPSSLSASVGDRVTITCRASQSISIYLNWYRQQPGKAPKLLIYAASSLQSGVPSRFSGSGSGTDFTLTISSLQPEDFATYFCQQTYSTPPWTFGQGTKVDIKTRTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGSGEGRGSLLTCGDVEENPGPMPPYTVVYFPVRGRCAALRMLLADQGQSWKEEVVTVETWQEGSLKASCLYGQLPKFQDGDLTLYQSNTILRHLGRTLGLYGKDQQEAALVDMVNDGVEDLRCKYISLIYTNYEAGKDDYVKALPGQLKPFETLLSQNQGGKTFIVGDQISFADYNLLDLLLIHEVLAPGCLDAFPLLSAYVGRLSARPKLKAFLASPEYVNLPINGNGKQ

SEQ ID NO: 66 - DNA, 2151 bp, synthetic construct
  misc_feature 1..2151: note = synthesized
  source 1..2151: mol_type = other DNA
atggaatttggcctgagctggctgtttctggtggcgattctgaaaggcgtgcagtgcgaggtgcagctgttccagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgccaggggtgttggccggggctttgactactggggccaggggaccacggtcaccgtctcctcaagtggcggtggctctggcggtggtgggtcgggtggcggcggatcagacatccagttgacccagtctccatcctccctgtctgcatctgtaggagacagagtcaccatcacttgccgggcaagtcagagcattagcatctatttaaattggtatcggcagcaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttacttctgtcaacagacttacagtacccctccgtggacgttcggccaagggaccaaagtggatatcaaaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatccggatctggagagggcagaggaagtcttctaacatgcggtgacgtggaggagaatcccggccctatgccgccctacaccgtggtctatttcccagttcgaggccgctgcgcggccctgcgcatgctgctggcagatcagggccagagctggaaggaggaggtggtgaccgtggagacgtggcaggagggctcactcaaagcctcctgcctatacgggcagctccccaagttccaggacggagacctcaccctgtaccagtccaataccatcctgcgtcacctgggccgcacccttgggctctatgggaaggaccagcaggaggcagccctggtggacatggtgaatgacggcgtggaggacctccgctgcaaatacatctccctcatctacaccaactatgaggcgggcaaggatgactatgtgaaggcactgcccgggcaactgaagccttttgagaccctgctgtcccagaaccagggaggcaagaccttcattgtgggagaccagatctccttcgctgactacaacctgctggacttgctgctgatccatgaggtcctagcccctggctgcctggatgcgttccccctgctctcagcatatgtggggcgcctcagtgcccggcccaagctcaaggccttcctggcctcccctgagtacgtgaacctccccatcaatggcaacgggaaacagtga

SEQ ID NO: 67 - protein, 240 aa, synthetic construct
  REGION 1..240: note = synthesized
QVQLVQSGGGVVQPGRSLRLSCSASGFTFNSHGMHWVRQAPGKGLEWVGVIWYDGSNAYYADSAKGRFTISRDNSKNSVYLQLNSLGVEDTALYYCAKSDRPHPMGFYYYMDVWGQGTTVTVSSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLVFGGGTK

SEQ ID NO: 68 - DNA, 720 bp, synthetic construct
  misc_feature 1..720: note = synthesized
  source 1..720: mol_type = other DNA
caggtgcagctggtgcagagcggcggcggcgtggtgcagccgggccgcagcctgcgcctgagctgcagcgcgagcggctttacctttaacagccatggcatgcattgggtgcgccaggcgccgggcaaaggcctggaatgggtgggcgtgatttggtatgatggcagcaacgcgtattatgcggatagcgcgaaaggccgctttaccattagccgcgataacagcaaaaacagcgtgtatctgcagctgaacagcctgggcgtggaagataccgcgctgtattattgcgcgaaaagcgatcgcccgcatccgatgggcttttattattatatggatgtgtggggccagggcaccaccgtgaccgtgagcagcggcggcggcggcagcggcggcggcggcagccagagcgcgctgacccagccggcgagcgtgagcggcagcccgggccagagcattaccattagctgcaccggcaccagcagcgatgtgggcggctataactatgtgagctggtatcagcagcatccgggcaaagcgccgaaactgatgatttatgatgtgagcaaccgcccgagcggcgtgagcaaccgctttagcggcagcaaaagcggcaacaccgcgagcctgaccattagcggcctgcaggcggaagatgaagcggattattattgcagcagctataccagcagcagcaccctggtgtttggcggcggcaccaaa